SEQUENCE LISTING (38 sequences)

SEQ ID NO: 1 - DNA, 1115 bp, Mus musculus
gaggctcaagtgcacccagcaccagctgatcaggacgcgcaaacatgagtccagggagagcttcatctgtgtctctgatgctgttgctgctgctgagcctggcggctacagtgaaggcagcagcgatcatccctcaaagctcagcgtgtccaaacactgaggccaaggacttcctccagaatgtgaaggtcaacctcaaagtctttaactcccttggcgcaaaagtgagctccagaaggccctcagactacctcaaccgttccacgtcaccctggactctccaccgcaatgaagaccctgatagatatccctctgtgatctgggaagctcagtgccgccaccagcgctgtgtcaatgcggagggaaagctggaccaccacatgaattctgttctcatccagcaagagatcctggtcctgaagagggagcctgagagctgccccttcactttcagggtcgagaagatgctggtgggtgtgggctgcacctgcgtggcctcgattgtccgccaggcagcctaaacagagacccgcggctgacccctaagaaacccccacgtttctcagcaaacttacttgcatttttaaaacagttcgtgctattgattttcagcaaggaatgtggattcagaggcagattcagaattgtctgccctccacaatgaaaagaaggtgtaaaggggtcccaaactgcttcgtgtttgtttttctgtggactttaaattatttgtgtatttacaatatcccaagataactttgaaggcgtaacttatttaatgaagtatctacattattattatgtttctttctgaagaagacaaaattcaagactcagaaattttattatttaaaaggtaagcctatatttatatgagctatttatgaatctatttatttttcttcagtatttgaagtattaagaacatgattttcagatctacctagggaagtcctaagtaagattaaatattaatggaaatttcagctttactatttggttgatttaaggttctctcctctgaatggggtgaaaaccaaacttagttttatgtttaataactttttaaattattgaagattcaaaaaattggataatttagctccctactctgtttt

SEQ ID NO: 2 - DNA, 477 bp, Mus musculus
atgagtccagggagagcttcatctgtgtctctgatgctgttgctgctgctgagcctggcggctacagtgaaggcagcagcgatcatccctcaaagctcagcgtgtccaaacactgaggccaaggacttcctccagaatgtgaaggtcaacctcaaagtctttaactcccttggcgcaaaagtgagctccagaaggccctcagactacctcaaccgttccacgtcaccctggactctccaccgcaatgaagaccctgatagatatccctctgtgatctgggaagctcagtgccgccaccagcgctgtgtcaatgcggagggaaagctggaccaccacatgaattctgttctcatccagcaagagatcctggtcctgaagagggagcctgagagctgccccttcactttcagggtcgagaagatgctggtgggtgtgggctgcacctgcgtggcctcgattgtccgccaggcagcctaa

SEQ ID NO: 3 - protein, 158 aa, Mus musculus
MSPGRASSVSLMLLLLLSLAATVKAAAIIPQSSACPNTEAKDFLQNVKVNLKVFNSLGAKVSSRRPSDYLNRSTSPWTLHRNEDPDRYPSVIWEAQCRHQRCVNAEGKLDHHMNSVLIQQEILVLKREPESCPFTFRVEKMLVGVGCTCVASIVRQAA

SEQ ID NO: 4 - DNA, 1874 bp, Homo sapiens
gaattccggcaggcacaaactcatccatccccagttgattggaagaaacaacgatgactcctgggaagacctcattggtgtcactgctactgctgctgagcctggaggccatagtgaaggcaggaatcacaatcccacgaaatccaggatgcccaaattctgaggacaagaacttcccccggactgtgatggtcaacctgaacatccataaccggaataccaataccaatcccaaaaggtcctcagattactacaaccgatccacctcaccttggaatctccaccgcaatgaggaccctgagagatatccctctgtgatctgggaggcaaagtgccgccacttgggctgcatcaacgctgatgggaacgtggactaccacatgaactctgtccccatccagcaagagatcctggtcctgcgcagggagcctccacactgccccaactccttccggctggagaagatactggtgtccgtgggctgcacctgtgtcaccccgattgtccaccatgtggcctaagagctctggggagcccacactccccaaagcagttagactatggagagccgacccagcccctcaggaaccctcatccttcaaagacagcctcatttcggactaaactcattagagttcttaaggcagtttgtccaattaaagcttcagaggtaacacttggccaagatatgagatctgaattacctttccctctttccaagaaggaaggtttgactgagtaccaatttgcttcttgtttacttttttaagggctttaagttatttatgtatttaatatgccctgagataactttggggtataagattccattttaatgaattacctactttattttgtttgtctttttaaagaagataagattctgggcttgggaattttattatttaaaaggtaaaacctgtatttatttgagctatttaaggatctatttatgtttaagtatttagaaaaaggtgaaaaagcactattatcagttctgcctaggtaaatgtaagatagaattaaatggcagtgcaaaatttctgagtctttacaacatacggatatagtatttcctcctctttgtttttaaaagttataacatggctgaaaagaaagattaaacctactttcatatgtattaatttaaattttgcaatttgttgaggttttacaagagatacagcaagtctaactctctgttccattaaacccttataataaaatccttctgtaataataaagtttcaaaagaaaatgtttatttgttctcattaaatgtattttagcaaactcagctcttccctattgggaagagttatgcaaattctcctataagcaaaacaaagcatgtctttgagtaacaatgacctggaaatacccaaaattccaagttctcgatttcacatgccttcaagactgaacaccgactaaggttttcatactattagccaatgctgtagacagaagcattttgataggaatagagcaaataagataatggccctgaggaatggcatgtcattattaaagatcatatggggaaaatgaaaccctccccaaaatacaagaagttctgggaggagacattgtcttcagactacaatgtccagtttctcccctagactcaggcttcctttggagattaaggcccctcagagatcaacagaccaacatttttctcttcctcaagcaacactcctagggcctggcttctgtctgatcaaggcaccacacaacccagaaaggagctgatggggcagaatgaactttaagtatgagaaaagttcagcccaagtaaaataaaaactcaatcacattcaattccagagtagtttcaagtttcacatcgtaaccattttcgcccggaattc

SEQ ID NO: 5 - DNA, 468 bp, Homo sapiens
atgactcctgggaagacctcattggtgtcactgctactgctgctgagcctggaggccatagtgaaggcaggaatcacaatcccacgaaatccaggatgcccaaattctgaggacaagaacttcccccggactgtgatggtcaacctgaacatccataaccggaataccaataccaatcccaaaaggtcctcagattactacaaccgatccacctcaccttggaatctccaccgcaatgaggaccctgagagatatccctctgtgatctgggaggcaaagtgccgccacttgggctgcatcaacgctgatgggaacgtggactaccacatgaactctgtccccatccagcaagagatcctggtcctgcgcagggagcctccacactgccccaactccttccggctggagaagatactggtgtccgtgggctgcacctgtgtcaccccgattgtccaccatgtggcctaa

SEQ ID NO: 6 - protein, 155 aa, Homo sapiens
MTPGKTSLVSLLLLLSLEAIVKAGITIPRNPGCPNSEDKNFPRTVMVNLNIHNRNTNTNPKRSSDYYNRSTSPWNLHRNEDPERYPSVIWEAKCRHLGCINADGNVDYHMNSVPIQQEILVLRREPPHCPNSFRLEKILVSVGCTCVTPIVHHVA

SEQ ID NO: 7 - DNA, 1019 bp, Bos taurus
ggtccatctcacagcgagcacaagttcatctgtctacagtgaactggaaggaactaccatggcttctatgagaacttcatctatgtcactgctactgcttctgagtctggtggctcttgtgaaggcaggagtcatcatcccacagagtccaggctgcccacctactgaggacaagaacttcccacagcatgtgagggtcaacctaaacatcgttaaccggagcacaaactccagaaggcccaccgattatcacaagcgctccacctcaccttggactctccaccgcaatgaggaccctgagaggtacccctctgtgatctgggaggccaagtgcagccactcaggctgtatcaatgctgaagggaaggtggaccatcacatgaactctgtcaccatccagcaagagatcctggtcctccgaagggagtctcagcactgccctcactccttccggctggagaagatgctggtggccgtgggctgcacctgcgtcacccccattgtccgccatttggcttaagagctttctgcctgacccctactccccaaattagttaggtttcctggggagtagacccagccccttttctaactaacatcatttatgtatttaataagccctgaaataactttggggcataagattccactttgatgtatcacatgctttattttctattcttttaagacaagtttccagatttgtgaatattattatttaaaaggtagaacctatatttatatgagctatttatggatctatttatgtttcattagtctttagaaaaaaggtgaaatatatgagtatctgttttgcctagggaaatcctagataggattaaatagcagttgaaaatgtctgagcttgtactacatgtggctatagaattttctcttgcttgtctctttggagtacatgacacagctgaaaagagactaaatttagtttcatatttattaacttaattttggaaattattgaggcattacaagagagacagcaagtctaactgctctat

SEQ ID NO: 8 - DNA, 462 bp, Bos taurus
atggcttctatgagaacttcatctatgtcactgctactgcttctgagtctggtggctcttgtgaaggcaggagtcatcatcccacagagtccaggctgcccacctactgaggacaagaacttcccacagcatgtgagggtcaacctaaacatcgttaaccggagcacaaactccagaaggcccaccgattatcacaagcgctccacctcaccttggactctccaccgcaatgaggaccctgagaggtacccctctgtgatctgggaggccaagtgcagccactcaggctgtatcaatgctgaagggaaggtggaccatcacatgaactctgtcaccatccagcaagagatcctggtcctccgaagggagtctcagcactgccctcactccttccggctggagaagatgctggtggccgtgggctgcacctgcgtcacccccattgtccgccatttggcttaa

SEQ ID NO: 9 - protein, 153 aa, Bos taurus
MASMRTSSMSLLLLLSLVALVKAGVIIPQSPGCPPTEDKNFPQHVRVNLNIVNRSTNSRRPTDYHKRSTSPWTLHRNEDPERYPSVIWEAKCSHSGCINAEGKVDHHMNSVTIQQEILVLRRESQHCPHSFRLEKMLVAVGCTCVTPIVRHLA

SEQ ID NO: 10 - DNA, 1202 bp, Sus scrofa
tccatctctcagcaagctccagctcatccatctgcagtacatcaggagaaactacgatgactcctgtgagatcctcgtccctgtcactgctgcttctgctgagcctggtggctctcgtgaaggcgggaatcatgatcccacaaagtccaggatgcccaaaaactgaggacaagaacttccctcagcatgtaagggtcaacttgaacatcttgaaccggagcacacctgccagacggccctcagattactccaaacgcttcacctcaccatggactctccaacgcaacgaggaccccgagaggtactcctccgtgatctgggaggccaagtgcagccactcgggctgtatcaatgctgaagggaaggaagatcatcacatgaactctgtccccatccagcaagagatcttggtcctgcgaagggagcctcgccactgccccaactccttccggctggagaaagtgatggtgacagtgggctgcacctgtgtcacccccatcgtccgccatatttcttaagagcttctagtctgacccctgctccccaaatcagttaggctttcaggggagtagacccagccccatttctgaccaatctcaccagggttcttagttcatccaatggaaggtccagaggtaacgttttggttctgaaatggaatctgaaccacccttccctttccccaagaaggaagttttaggctgagtaccaattgtcgtcttgtttgcttttctaagggttttaagttatttatgtatttaatagactctgagataacttcagggcataagattccattttaatgaattacatgccttatttggtatttttttataagatgtgattccagacttgggaattttattatttaaaaggtaaaacctatatttatttgaactatttatggatctatttatctcttctgtctttagagaaaggtaaaatatatgagtatctgtgctgcctagagaaatcctatataggattaaatagcgattgaaaatttctcaggattttctcttcattgtctcttaggcatgcacgacatggctgaaataaagactaaacctagttttatatttattaacttaattttgtatattattgagttttcacaagagagacagcaagttgctctgtaaactcttgtaacaaaaacaattggtaataataaagtttggaaagaaaaaaaaaaaaaaaaa

SEQ ID NO: 11 - DNA, 462 bp, Sus scrofa
atgactcctgtgagatcctcgtccctgtcactgctgcttctgctgagcctggtggctctcgtgaaggcgggaatcatgatcccacaaagtccaggatgcccaaaaactgaggacaagaacttccctcagcatgtaagggtcaacttgaacatcttgaaccggagcacacctgccagacggccctcagattactccaaacgcttcacctcaccatggactctccaacgcaacgaggaccccgagaggtactcctccgtgatctgggaggccaagtgcagccactcgggctgtatcaatgctgaagggaaggaagatcatcacatgaactctgtccccatccagcaagagatcttggtcctgcgaagggagcctcgccactgccccaactccttccggctggagaaagtgatggtgacagtgggctgcacctgtgtcacccccatcgtccgccatatttcttaa

SEQ ID NO: 12 - protein, 153 aa, Sus scrofa
MTPVRSSSLSLLLLLSLVALVKAGIMIPQSPGCPKTEDKNFPQHVRVNLNILNRSTPARRPSDYSKRFTSPWTLQRNEDPERYSSVIWEAKCSHSGCINAEGKEDHHMNSVPIQQEILVLRREPRHCPNSFRLEKVMVTVGCTCVTPIVRHIS

SEQ ID NO: 13 - DNA, 1212 bp, Canis familiaris
agaaactacaatgactctggtgacaacttcatccatgttccagtcactgctactgcttctgagcctggtggctatcataaaggcaggaatagcattcccacaaaatccaggatgccgaaatactgaggacaagaactttcctcagcatgtgaaggtcaatctaaatatccttaaccggaatacgaactccagaaggccctcagattactacaaccgatctacctcaccttggaatctgcaccgcaatgaggaccctgagagatacccctctgtgatctgggaggcgaagtgccgccacttgggctgtgtcaataatgaagggaacataaactaccacatgaactccgtccccatccagcaagagatcctggttctgcgaagggagtcgcagcactgcccccactccttccggctagagaagatgctggtggctgtgggctgcacctgtgtcacccctattgttcgccatgtggcttaagagcttctgatctaatcccagctccccagagcagactttctgggaaagtagacccagccccatttctgactaaactcattagggttcttaaactagttcatccaattgaagtttcagaggtaacacttttgtgcagagataaaatctgaatgagccttccctctccccaaagatgaaagttttcactgagtaccactttgctttttattgtgcttttctaagggctttaagttatttatgtatttaatagaccctgagataacttgggggccaaagtttccattttaatgaattacatgccttattttgtacttttttaagaccagattccagacttgataattttattatttaacaggtaaaatctctatttatatgaactatttatggatctatttatatttcttaggtctttatagaaaggtgaaagagtgtgattatctgttctgcctagggaaatcctacagagaattcaatggcgggttgaaaatttctgaggtttgtattacacatttgataaaggattctttttctctttgtgctttacaagtggatgacatggctattaaaaaatgaactaaagactaaacctagtttcatatttattaattttgtaaattgttgaggtttcacaagagagatggcaagtttaactgctctgttaaaccctcataataaaaaaattttgtaataaagtttggaaagaaaaaaaaaaaaaaaaaaaaaaaaaaaaa

SEQ ID NO: 14 - DNA, 468 bp, Canis familiaris
atgactctggtgacaacttcatccatgttccagtcactgctactgcttctgagcctggtggctatcataaaggcaggaatagcattcccacaaaatccaggatgccgaaatactgaggacaagaactttcctcagcatgtgaaggtcaatctaaatatccttaaccggaatacgaactccagaaggccctcagattactacaaccgatctacctcaccttggaatctgcaccgcaatgaggaccctgagagatacccctctgtgatctgggaggcgaagtgccgccacttgggctgtgtcaataatgaagggaacataaactaccacatgaactccgtccccatccagcaagagatcctggttctgcgaagggagtcgcagcactgcccccactccttccggctagagaagatgctggtggctgtgggctgcacctgtgtcacccctattgttcgccatgtggcttaa

SEQ ID NO: 15 - protein, 155 aa, Canis familiaris
MTLVTTSSMFQSLLLLLSLVAIIKAGIAFPQNPGCRNTEDKNFPQHVKVNLNILNRNTNSRRPSDYYNRSTSPWNLHRNEDPERYPSVIWEAKCRHLGCVNNEGNINYHMNSVPIQQEILVLRRESQHCPHSFRLEKMLVAVGCTCVTPIVRHVA

SEQ ID NO: 16 - DNA, 580 bp, Gallus gallus
tgtctccgatcccttgttctcctctgaccactgctgttggtgttgctggccatgttgtcagccagcatttctgcccatgggaaggtgatacggccaggactcgagccagagagcctcttcaagaaagcagatgctggatgcctaacccaaaaagatggaaaattcccccaaactgtgagagtgaacataagcatcagcaacatgaaccaggatactaaagtgacccttgatatcagcaaacgctcactggctccatgggattacaggatcgatgaggaccacaaccgcttcccccgcttggttgctgatgcccagtgccgccattccaggtgcgtgaactcggctgggcagctggaccacagcgtcaactccgtgcccatcaaacaggagatcctcgtcctccgccgcgagccgaagggctgccagcactcgtaccgcctggagaagaaaatgatcaccgtgggctgcacatgtgtcaccccactgatccagcaccaggcttaaagggagcccacagcacaggagtggggagctgcaggagaactgccttgcctaacagcgatgaaaatccacagagctg

SEQ ID NO: 17 - DNA, 453 bp, Gallus gallus
atgttgtcagccagcatttctgcccatgggaaggtgatacggccaggactcgagccagagagcctcttcaagaaagcagatgctggatgcctaacccaaaaagatggaaaattcccccaaactgtgagagtgaacataagcatcagcaacatgaaccaggatactaaagtgacccttgatatcagcaaacgctcactggctccatgggattacaggatcgatgaggaccacaaccgcttcccccgcttggttgctgatgcccagtgccgccattccaggtgcgtgaactcggctgggcagctggaccacagcgtcaactccgtgcccatcaaacaggagatcctcgtcctccgccgcgagccgaagggctgccagcactcgtaccgcctggagaagaaaatgatcaccgtgggctgcacatgtgtcaccccactgatccagcaccaggcttaa

SEQ ID NO: 18 - protein, 150 aa, Gallus gallus
MLSASISAHGKVIRPGLEPESLFKKADAGCLTQKDGKFPQTVRVNISISNMNQDTKVTLDISKRSLAPWDYRIDEDHNRFPRLVADAQCRHSRCVNSAGQLDHSVNSVPIKQEILVLRREPKGCQHSYRLEKKMITVGCTCVTPLIQHQA

SEQ ID NO: 19 - DNA, 498 bp, Artificial Sequence
  Optimized Mouse IL-17A Nucleotide Sequence
ggatccgccaccatgtctccaggtcgggcttccagtgtctctctgatgctgctgctgctgctctccctcgccgccacagtgaaggctgccgctattattcctcagagctccgcctgcccaaacactgaggctaaggacttcctgcagaacgtgaaggtcaatctgaaagtgtttaattccctcggcgctaaagtctctagtaggagaccctctgattacctgaacaggtcaaccagcccttggacactccaccgaaatgaggaccccgataggtatcctagcgtgatctgggaagcacagtgccggcatcagaggtgcgtgaacgccgagggaaagctggaccaccatatgaatagtgtgctcatccagcaggaaattctggtcctcaaaagagagccagaatcctgtcccttcacctttcgggtggaaaagatgctggtcggggtgggttgtacttgcgtggcttccattgtgagacaggctgcctgataactcgag

SEQ ID NO: 20 - DNA, 474 bp, Artificial Sequence
  Optimized Mouse IL-17A Coding Sequence
atgtctccaggtcgggcttccagtgtctctctgatgctgctgctgctgctctccctcgccgccacagtgaaggctgccgctattattcctcagagctccgcctgcccaaacactgaggctaaggacttcctgcagaacgtgaaggtcaatctgaaagtgtttaattccctcggcgctaaagtctctagtaggagaccctctgattacctgaacaggtcaaccagcccttggacactccaccgaaatgaggaccccgataggtatcctagcgtgatctgggaagcacagtgccggcatcagaggtgcgtgaacgccgagggaaagctggaccaccatatgaatagtgtgctcatccagcaggaaattctggtcctcaaaagagagccagaatcctgtcccttcacctttcgggtggaaaagatgctggtcggggtgggttgtacttgcgtggcttccattgtgagacaggctgcc

SEQ ID NO: 21 - protein, 158 aa, Artificial Sequence; identical to SEQ ID NO: 3
  Amino Acid Sequence Encoded by Optimized Mouse IL-17A Nucleic Acid Sequences

SEQ ID NO: 22 - DNA, 489 bp, Artificial Sequence
  Optimized Human IL-17A Nucleotide Sequence
ggatccgccaccatgacccctggaaaaacaagtctggtgtctctgctgctgctgctgtctctggaggcaatcgtcaaggctggaatcacaatcccacggaacccaggctgccccaattccgaggacaagaacttccctagaactgtgatggtcaacctgaatatccacaacaggaataccaacacaaatcccaaacgaagctccgactactataatcggtctaccagtccttggaacctgcataggaatgaggaccccgaacgctacccaagtgtgatctgggaagccaagtgccgccacctgggctgtattaacgctgacgggaatgtggattatcatatgaattcagtcccaatccagcaggagattctggtcctgcggagagaaccccctcactgtcccaacagctttcgactggaaaaaatcctggtgtccgtgggctgtacctgcgtgactccaatcgtccatcatgtggcttgataactcgag

SEQ ID NO: 23 - DNA, 465 bp, Artificial Sequence
  Optimized Human IL-17A Coding Sequence
atgacccctggaaaaacaagtctggtgtctctgctgctgctgctgtctctggaggcaatcgtcaaggctggaatcacaatcccacggaacccaggctgccccaattccgaggacaagaacttccctagaactgtgatggtcaacctgaatatccacaacaggaataccaacacaaatcccaaacgaagctccgactactataatcggtctaccagtccttggaacctgcataggaatgaggaccccgaacgctacccaagtgtgatctgggaagccaagtgccgccacctgggctgtattaacgctgacgggaatgtggattatcatatgaattcagtcccaatccagcaggagattctggtcctgcggagagaaccccctcactgtcccaacagctttcgactggaaaaaatcctggtgtccgtgggctgtacctgcgtgactccaatcgtccatcatgtggct

SEQ ID NO: 24 - protein, 155 aa, Artificial Sequence; identical to SEQ ID NO: 6
  Amino Acid Sequence Encoded by Optimized Human IL-17A Nucleic Acid Sequences

SEQ ID NO: 25 - DNA, 483 bp, Artificial Sequence
  Optimized Cow IL-17A Nucleotide Sequence
ggatccgccaccatggctagtatgcgaacttcctctatgtcactgctgctgctgctgtctctggtcgctctggtcaaggccggggtcattattcctcagagccccggctgccctcccacagaggacaagaacttccctcagcacgtgagggtcaacctgaatatcgtgaaccgctcaactaatagccggaggcctaccgactaccacaaaagaagcacctccccatggacactgcatcgaaatgaggaccccgaacggtatcccagtgtgatctgggaggccaagtgctctcatagtggctgtattaacgctgaagggaaagtggatcaccatatgaattccgtcaccatccagcaggagattctggtcctgcgcagagaatcccagcactgtccccattcttttcgactggaaaaaatgctggtcgctgtgggctgtacttgcgtgactcctattgtgagacacctggcttgataactcgag

SEQ ID NO: 26 - DNA, 459 bp, Artificial Sequence
  Optimized Cow IL-17A Coding Sequence
atggctagtatgcgaacttcctctatgtcactgctgctgctgctgtctctggtcgctctggtcaaggccggggtcattattcctcagagccccggctgccctcccacagaggacaagaacttccctcagcacgtgagggtcaacctgaatatcgtgaaccgctcaactaatagccggaggcctaccgactaccacaaaagaagcacctccccatggacactgcatcgaaatgaggaccccgaacggtatcccagtgtgatctgggaggccaagtgctctcatagtggctgtattaacgctgaagggaaagtggatcaccatatgaattccgtcaccatccagcaggagattctggtcctgcgcagagaatcccagcactgtccccattcttttcgactggaaaaaatgctggtcgctgtgggctgtacttgcgtgactcctattgtgagacacctggct

SEQ ID NO: 27 - protein, 153 aa, Artificial Sequence; identical to SEQ ID NO: 9
  Amino Acid Sequence Encoded by Optimized Cow IL-17A Nucleic Acid Sequences

SEQ ID NO: 28 - DNA, 483 bp, Artificial Sequence
  Optimized Pig IL-17A Nucleotide Sequence
ggatccgccaccatgacaccagtccgctcctccagcctcagcctcctcctgctcctctctctcgtcgccctcgtgaaagccggcattatgattccacagagccctggctgcccaaagaccgaggacaagaacttcccacagcacgtgagggtcaacctgaatatcctgaacagatccaccccagctagacgcccctctgactacagcaagcgattcaccagcccatggaccctgcagagaaacgaagacccagagaggtactcctcagtgatctgggaagccaagtgcagccactccggatgcatcaacgccgaaggcaaggaggaccaccacatgaactccgtgccaatccagcaggaaatcctcgtgctgagaagggaaccaagacactgccctaactccttcaggctggaaaaagtcatggtcacagtcggctgcacctgcgtcacacctattgtccgccacatctcttgataactcgag

SEQ ID NO: 29 - DNA, 459 bp, Artificial Sequence
  Optimized Pig IL-17A Coding Sequence
atgacaccagtccgctcctccagcctcagcctcctcctgctcctctctctcgtcgccctcgtgaaagccggcattatgattccacagagccctggctgcccaaagaccgaggacaagaacttcccacagcacgtgagggtcaacctgaatatcctgaacagatccaccccagctagacgcccctctgactacagcaagcgattcaccagcccatggaccctgcagagaaacgaagacccagagaggtactcctcagtgatctgggaagccaagtgcagccactccggatgcatcaacgccgaaggcaaggaggaccaccacatgaactccgtgccaatccagcaggaaatcctcgtgctgagaagggaaccaagacactgccctaactccttcaggctggaaaaagtcatggtcacagtcggctgcacctgcgtcacacctattgtccgccacatctct

SEQ ID NO: 30 - protein, 153 aa, Artificial Sequence; identical to SEQ ID NO: 12
  Amino Acid Sequence Encoded by Optimized Pig IL-17A Nucleic Acid Sequences

SEQ ID NO: 31 - DNA, 489 bp, Artificial Sequence
  Optimized Dog IL-17A Nucleotide Sequence
ggatccgccaccatgaccctggtgacaacctcctccatgttccagtccctgctgctgctgctgagcctggtggccatcatcaaggccggcatcgccttcccccagaaccccggctgcaggaacaccgaggacaagaacttcccccagcacgtgaaggtgaacctgaacatcctgaacaggaacaccaactcccgccggcccagcgactactataaccggtccacaagcccctggaacctgcaccggaacgaggaccccgagaggtacccctccgtgatctgggaggccaagtgcaggcacctgggctgcgtgaacaacgagggcaacatcaactatcacatgaacagcgtgcccatccagcaggagatcctggtgctgaggcgcgagtcccagcactgtccccacagcttccggctggagaagatgctggtggccgtgggctgtacctgcgtgacccccatcgtgaggcacgtggcctgataactcgag

SEQ ID NO: 32 - DNA, 465 bp, Artificial Sequence
  Optimized Dog IL-17A Coding Sequence
atgaccctggtgacaacctcctccatgttccagtccctgctgctgctgctgagcctggtggccatcatcaaggccggcatcgccttcccccagaaccccggctgcaggaacaccgaggacaagaacttcccccagcacgtgaaggtgaacctgaacatcctgaacaggaacaccaactcccgccggcccagcgactactataaccggtccacaagcccctggaacctgcaccggaacgaggaccccgagaggtacccctccgtgatctgggaggccaagtgcaggcacctgggctgcgtgaacaacgagggcaacatcaactatcacatgaacagcgtgcccatccagcaggagatcctggtgctgaggcgcgagtcccagcactgtccccacagcttccggctggagaagatgctggtggccgtgggctgtacctgcgtgacccccatcgtgaggcacgtggcc

SEQ ID NO: 33 - protein, 155 aa, Artificial Sequence; identical to SEQ ID NO: 15
  Amino Acid Sequence Encoded by Optimized Dog IL-17A Nucleic Acid Sequences

SEQ ID NO: 34 - DNA, 474 bp, Artificial Sequence
  Optimized Chicken IL-17A Nucleotide Sequence
ggatccgccaccatgctgagcgcaagtattagtgcccacgggaaagtgattcgccctggactggaacctgaaagcctgtttaagaaagccgatgccggttgcctgactcagaaggacggcaaattcccccagaccgtgagagtgaacatcagcatttccaacatgaatcaggataccaaggtgacactggacatctctaaacgctcactggcaccatgggattaccggattgatgaggaccacaataggtttccaaggctggtggcagacgctcagtgcagacactccaggtgcgtgaactcagccggccagctggatcatagtgtgaatagcgtgcccatcaagcaggagattctggtgctgagaagggaacctaaaggatgtcagcattcttatagactggagaagaagatgattaccgtgggttgtacttgtgtgactcctctgatccagcaccaggcatgataactcgag

SEQ ID NO: 35 - DNA, 450 bp, Artificial Sequence
  Optimized Chicken IL-17A Coding Sequence
atgctgagcgcaagtattagtgcccacgggaaagtgattcgccctggactggaacctgaaagcctgtttaagaaagccgatgccggttgcctgactcagaaggacggcaaattcccccagaccgtgagagtgaacatcagcatttccaacatgaatcaggataccaaggtgacactggacatctctaaacgctcactggcaccatgggattaccggattgatgaggaccacaataggtttccaaggctggtggcagacgctcagtgcagacactccaggtgcgtgaactcagccggccagctggatcatagtgtgaatagcgtgcccatcaagcaggagattctggtgctgagaagggaacctaaaggatgtcagcattcttatagactggagaagaagatgattaccgtgggttgtacttgtgtgactcctctgatccagcaccaggca

SEQ ID NO: 36 - protein, 150 aa, Artificial Sequence; identical to SEQ ID NO: 18
  Amino Acid Sequence Encoded by Optimized Chicken IL-17A Nucleic Acid Sequences

SEQ ID NO: 37 - DNA, 54 bp, Artificial Sequence
  IgE leader sequence
atggactggacttggattctgttcctggtcgccgccgcaactcgcgtgcatagc

SEQ ID NO: 38 - protein, 18 aa, Artificial Sequence
  IgE leader sequence
MDWTWILFLVAAATRVHS

(19) United States
(12) Patent Application Publication
(10) Pub. No.: US 2017/0182154 A1
(43) Pub. Date: Jun. 29, 2017

(54) VACCINES WITH INTERLEUKIN-17 AS AN ADJUVANT

(71) Applicant: BEIJING ADVACCINE BIOTECHNOLOGY CO. LTD, Beijing (CN)

(72) Inventors: Bin WANG, Beijing (CN); Jin JIN, Beijing (CN); Xiaoping XIE, Beijing (CN); Zhonghuai HE, Beijing (CN); Qingling YU, Beijing (CN)

(21) Appl. No.: 15/118,211

(22) PCT Filed: Feb. 10, 2015
(86) PCT No.: PCT/CN2015/072619

§ 371 Date: Aug. 11, 2016

Related U.S. Application Data
(60) Provisional application No. 62/108,596, filed on Jan. 28, 2015.
(60) Provisional application No. 61/938,315, filed on Feb. 11, 2014.

(30) Foreign Application Priority Data
Jan. 22, 2014 (CN) XW2014000032

(51) Int. Cl. A61K 39/39; A61K 39/12; A61K 39/05; A61K 39/145

(52) U.S. Cl. CPC A61K 39/39; A61K 39/145; A61K 39/12; A61K 39/05; C12N 2760/16134; C12N 2770/10034; C12N 2750/10034; C12N 2770/32134; A61K 2039/525

(57) ABSTRACT

Disclosed herein is a vaccine comprising an antigen and IL-17. Also disclosed herein is a method for increasing an immune response in a subject. The method may comprise administering the vaccine to the subject in need thereof.

### TECHNICAL FIELD

The present invention relates to vaccines comprising an antigen and IL-17, and methods of administering such vaccines.

### BACKGROUND

Vaccines are used to stimulate an immune response in an individual to provide protection against and/or treatment for a particular disease. Some vaccines include an antigen to induce the immune response. Some antigens elicit a strong immune response while other antigens elicit a weak immune response. A weak immune response to an antigen can be strengthened by including an adjuvant in the vaccine. Adjuvants come in many different forms, for example, aluminum salts, oil emulsions, sterile constituents of bacteria or other pathogens, cytokines, and so forth.

Cytokines are proteins made by cells that affect the behavior of other cells, and unlike many adjuvants, can modulate specific immune responses. One such cytokine is Interleukin-17 (IL-17). IL-17 is a family of cytokines that signal through receptors to induce the production of anti-microbial peptides and pro-inflammatory cytokines such as interferon-γ (IFN-γ). The IL-17 family of cytokines induces adaptive immune responses such as T helper type 2 (Th2) immune responses and innate immune responses such as the expansion and recruitment of neutrophils. Accordingly, the IL-17 family of cytokines can bridge the adaptive and innate immune responses.

Vaccines are also administered in many different ways (e.g., injection, orally, etc.) into many different tissues (e.g., intramuscular, nasal, etc.). Not all delivery methods, however, are equal. Some delivery methods allow for greater compliance within a population of individuals while other delivery methods may affect immunogenicity and/or safety of the vaccine. Accordingly, a need remains in the art for the development of safe and more effective adjuvants that increase immune responses to the antigen.

### SUMMARY

The present invention is directed to a vaccine comprising an antigen and IL-17.

The present invention is also directed to a method for inducing or increasing an immune response in a subject. The method can comprise administering an antigen and IL-17 to the subject in need thereof. The present invention also relates, generally, to the use of IL-17 as an adjuvant.

### BRIEF DESCRIPTION OF THE DRAWINGS

FIG. 1 shows (A) a graph plotting mouse group vs. cytokine level; (B) a graph plotting days post infection (dpi) vs. percent survival; and (C) a graph plotting dpi vs. percent survival.

FIG. 2 shows (A) a graph plotting mouse group vs. cytokine level; (B) a graph plotting mouse group vs. cytokine level; and (C) a graph plotting days post infection (dpi) vs. percent survival.

FIG. 3 shows (A) a graph plotting cell type vs. percent interferon-gamma (IFN-γ) producing cells; (B) a graph plotting days post infection (dpi) vs. percent survival; and (C) a graph plotting dpi vs. percent survival.

FIG. 4 shows (A) an illustration that depicts the experimental scheme for adoptive transfer of CD8⁺ T cells into recipient mice; (B) a graph plotting days post infection (dpi) vs. percent survival; and (C) a graph plotting dpi vs. percent survival.

FIG. 5 shows (A) a graph plotting mouse type vs. percent specific lysis; and (B) a graph plotting CD8⁺ T cell population vs. percent specific lysis.

FIG. 6 shows (A) the mRNA nucleotide sequence; (B) the coding nucleotide sequence; and (C) the amino acid sequence for Mus musculus (mouse) IL-17A.

FIG. 7 shows (A) the mRNA nucleotide sequence; (B) the coding nucleotide sequence; and (C) the amino acid sequence for Homo sapiens (human) IL-17A.

FIG. 8 shows (A) the mRNA nucleotide sequence; (B) the coding nucleotide sequence; and (C) the amino acid sequence for Bos taurus (cow) IL-17A.

FIG. 9 shows (A) the mRNA nucleotide sequence; (B) the coding nucleotide sequence; and (C) the amino acid sequence for Sus scrofa (pig) IL-17A.

FIG. 10 shows (A) the mRNA nucleotide sequence; (B) the coding nucleotide sequence; and (C) the amino acid sequence for Canis lupus familiaris (dog) IL-17A.

FIG. 11 shows (A) the mRNA nucleotide sequence; (B) the coding nucleotide sequence; and (C) the amino acid sequence for Gallus gallus (chicken) IL-17A.

FIG. 12 shows (A) the optimized mouse IL-17A nucleotide sequence, in which the underlined sequence contains a BamHI site (GGA TCC) and a Kozak sequence (GCC ACC) and the double underlined sequence contains the stop codons TGA and TAA and a XhoI site (CTC GAG); (B) the optimized mouse IL-17A coding nucleotide sequence; and (C) the mouse IL-17A amino acid sequence encoded by the optimized nucleotide sequences of FIGS. 12A and 12B (i.e., SEQ ID NOS:19 and 20).

FIG. 13 shows (A) the optimized human IL-17A nucleotide sequence, in which the underlined sequence contains a BamHI site (GGA TCC) and a Kozak sequence (GCC ACC) and the double underlined sequence contains the stop codons TGA and TAA and a XhoI site (CTC GAG); (B) the optimized human IL-17A coding nucleotide sequence; and (C) the human IL-17A amino acid sequence encoded by the optimized nucleotide sequences of FIGS. 13A and 13B (i.e., SEQ ID NOS:22 and 23).

FIG. 14 shows (A) the optimized cow IL-17A nucleotide sequence, in which the underlined sequence contains a BamHI site (GGA TCC) and a Kozak sequence (GCC ACC) and the double underlined sequence contains the stop codons TGA and TAA and a XhoI site (CTC GAG); (B) the optimized cow IL-17A coding nucleotide sequence; and (C) the cow IL-17A amino acid sequence encoded by the optimized nucleotide sequences of FIGS. 14A and 14B (i.e., SEQ ID NOS:25 and 26).

FIG. 15 shows (A) the optimized pig IL-17A nucleotide sequence, in which the underlined sequence contains a BamHI site (GGA TCC) and a Kozak sequence (GCC ACC) and the double underlined sequence contains the stop codons TGA and TAA and a XhoI site (CTC GAG); (B) the optimized pig IL-17A coding nucleotide sequence; and (C) the pig IL-17A amino acid sequence encoded by the optimized nucleotide sequences of FIGS. 15A and 15B (i.e., SEQ ID NOS:28 and 29).

FIG. 16 shows (A) the optimized dog IL-17A nucleotide sequence, in which the underlined sequence contains a BamHI site (GGA TCC) and a Kozak sequence (GCC ACC) and the double underlined sequence contains the stop codons TGA and TAA and a XhoI site (CTC GAG); (B) the optimized dog IL-17A coding nucleotide sequence; and (C) the dog IL-17A amino acid sequence encoded by the optimized nucleotide sequences of FIGS. 16A and 16B (i.e., SEQ ID NOS:31 and 32).

FIG. 17 shows (A) the optimized chicken IL-17A nucleotide sequence, in which the underlined sequence contains a BamHI site (GGA TCC) and a Kozak sequence (GCC ACC) and the double underlined sequence contains the stop codons TGA and TAA and a XhoI site (CTC GAG); (B) the optimized chicken IL-17A coding nucleotide sequence; and (C) the chicken IL-17A amino acid sequence encoded by the optimized nucleotide sequences of FIGS. 17A and 17B (i.e., SEQ ID NOS:34 and 35).

FIG. 18 shows a graph plotting immunization group versus antibody titer.

FIG. 19 shows a graph plotting immunization group versus antibody titer.

FIG. 20 shows graphs plotting time (days) versus temperature for animals vaccinated with (A) live PRRSV vaccine; (B) killed PRRSV vaccine; (C) IL-17A (3 μg)+PRRSV killed vaccine; and (D) IL-17A (9 μg)+PRRSV vaccine.

FIG. 21 shows a graph plotting immunization group versus antibody titer.

FIG. 22 shows graphs plotting antibody levels for each immunization group at 21, 35, 48, and 63 days.

FIG. 23 shows a graph plotting immunization group versus antibody titer.

### DETAILED DESCRIPTION

The present invention relates to a vaccine that can be used to induce or increase an immune response to an antigen in a subject by using IL-17 as an adjuvant. The invention also relates to the use of IL-17 as an adjuvant, to stimulate an immune response in a subject. The invention also relates to methods for vaccinating or inducing or increasing an immune response in a subject by administering to the subject an antigen an IL-17. IL-17 can be administered together with or separately from the antigen. As illustrated in the present invention, a strong immune response is elicited in the subject when IL-17 is administered before the antigen as compared to a vaccine comprising the antigen alone. Such separate administration readies the immune system for exposure to the antigen, thereby providing an increased immune response to the antigen in the subject.

The vaccine of the present invention can increase the immune response to the antigen in the subject by increasing the CD8⁺ T cell response as compared to the vaccine not including IL-17. This increased CD8⁺ T cell response has cytolytic activity and secretes the anti-viral cytokine interferon-gamma (IFN-γ). Accordingly, IL-17 may further augment the immune response to viral antigens, for example, influenza viral antigens.

The vaccine of the present invention can also increase the immune response to the antigen in the subject by increasing the antibody titer and duration of the antibody response to the antigen as compared to the vaccine not including IL-17.

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

“Adjuvant” as used herein means any molecule, which may be added to a vaccine, that enhances the immunogenicity of an antigen.

“Coding sequence” or “encoding nucleic acid” as used herein means the nucleic acids (RNA or DNA molecule) that comprise a nucleotide sequence which encodes a protein. The coding sequence can further include initiation and termination signals operably linked to regulatory elements including a promoter and polyadenylation signal capable of directing expression in the cells of an individual or mammal to which the nucleic acid is administered.

“Complement” or “complementary” as used herein means a nucleic acid can mean Watson-Crick (e.g., A-T/U and C-G) or Hoogsteen base pairing between nucleotides or nucleotide analogs of nucleic acid molecules.

“Electroporation,” “electro-permeabilization,” or “electro-kinetic enhancement” (“EP”) as used interchangeably herein means the use of a transmembrane electric field pulse to induce microscopic pathways (pores) in a bio-membrane; their presence allows biomolecules such as plasmids, oligonucleotides, siRNA, drugs, ions, and water to pass from one side of the cellular membrane to the other.

“Fragment” or “immunogenic fragment” as used herein means a nucleic acid sequence or a portion thereof that encodes a polypeptide capable of eliciting an immune response in a mammal. The fragments can be DNA fragments selected from at least one of the various nucleotide sequences that encode protein fragments set forth below. Fragments can comprise at least 10%, at least 20%, at least 30%, at least 40%, at least 50%, at least 60%, at least 70%, at least 80%, at least 90%, or at least 95% of one or more of the nucleic acid sequences set forth below. In some embodiments, fragments can comprise at least 20 nucleotides or more, at least 30 nucleotides or more, at least 40 nucleotides or more, at least 50 nucleotides or more, at least 60 nucleotides or more, at least 70 nucleotides or more, at least 80 nucleotides or more, at least 90 nucleotides or more, at least 100 nucleotides or more, at least 150 nucleotides or more, at least 200 nucleotides or more, at least 250 nucleotides or more, at least 300 nucleotides or more, at least 350 nucleotides or more, at least 400 nucleotides or more, at least 450 nucleotides or more, at least 500 nucleotides or more, at least 550 nucleotides or more, at least 600 nucleotides or more, at least 650 nucleotides or more, at least 700 nucleotides or more, at least 750 nucleotides or more, at least 800 nucleotides or more, at least 850 nucleotides or more, at least 900 nucleotides or more, at least 950 nucleotides or more, or at least 1000 nucleotides or more of at least one of the nucleic acid sequences set forth below.

Fragment or immunogenic fragment as used herein also means a polypeptide sequence or a portion thereof that is capable of eliciting an immune response in a mammal. The fragments can be polypeptide fragments selected from at least one of the various amino acid sequence set forth below. Fragments can comprise at least 10%, at least 20%, at least 30%, at least 40%, at least 50%, at least 60%, at least 70%, at least 80%, at least 90%, or at least 95% of one or more of the proteins set forth below. In some embodiments, fragments can comprise at least 20 amino acids or more, at least 30 amino acids or more, at least 40 amino acids or more, at least 50 amino acids or more, at least 60 amino acids or more, at least 70 amino acids or more, at least 80 amino acids or more, at least 90 amino acids or more, at least 100 amino acids or more, at least 110 amino acids or more, at least 120 amino acids or more, at least 130 amino acids or more, at least 140 amino acids or more, at least 150 amino acids or more, at least 160 amino acids or more, at least 170 amino acids or more, at least 180 amino acids or more, at least 190 amino acids or more, at least 200 amino acids or more, at least 210 amino acids or more, at least 220 amino acids or more, at least 230 amino acids or more, or at least 240 amino acids or more of at least one of the proteins set forth below.

“Genetic construct” as used herein refers to the DNA or RNA molecules that comprise a nucleotide sequence which encodes a protein. The coding sequence includes initiation and termination signals operably linked to regulatory elements including a promoter and polyadenylation signal capable of directing expression in the cells of the individual to whom the nucleic acid molecule is administered. As used herein, the term “expressible form” refers to gene constructs that contain the necessary regulatory elements operable linked to a coding sequence that encodes a protein such that when present in the cell of the individual, the coding sequence will be expressed.

“Identical” or “identity” as used herein in the context of two or more nucleic acids or polypeptide sequences, means that the sequences have a specified percentage of residues that are the same over a specified region. The percentage can be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) can be considered equivalent. Identity can be performed manually or by using a computer sequence algorithm such as BLAST or BLAST 2.0.

“Immune response” as used herein means the activation of a host's immune system, e.g., that of a mammal, in response to the introduction of antigen. The immune response can be in the form of a cellular or humoral response, or both.

“Nucleic acid” or “oligonucleotide” or “polynucleotide” as used herein means at least two nucleotides covalently linked together. The depiction of a single strand also defines the sequence of the complementary strand. Thus, a nucleic acid also encompasses the complementary strand of a depicted single strand. Many variants of a nucleic acid can be used for the same purpose as a given nucleic acid. Thus, a nucleic acid also encompasses substantially identical nucleic acids and complements thereof. A single strand provides a probe that can hybridize to a target sequence under stringent hybridization conditions. Thus, a nucleic acid also encompasses a probe that hybridizes under stringent hybridization conditions.

Nucleic acids can be single stranded or double stranded, or can contain portions of both double stranded and single stranded sequence. The nucleic acid can be DNA, both genomic and cDNA, RNA, or a hybrid, where the nucleic acid can contain combinations of deoxyribo- and ribo-nucleotides, and combinations of bases including uracil, adenine, thymine, cytosine, guanine, inosine, xanthine hypoxanthine, isocytosine and isoguanine. Nucleic acids can be obtained by chemical synthesis methods or by recombinant methods.

“Operably linked” as used herein means that expression of a gene is under the control of a promoter with which it is spatially connected. A promoter can be positioned 5′ (upstream) or 3′ (downstream) of a gene under its control. The distance between the promoter and a gene can be approximately the same as the distance between that promoter and the gene it controls in the gene from which the promoter is derived. As is known in the art, variation in this distance can be accommodated without loss of promoter function.

A “peptide,” “protein,” or “polypeptide” as used herein can mean a linked sequence of amino acids and can be natural, synthetic, or a modification or combination of natural and synthetic.

“Promoter” as used herein means a synthetic or naturally-derived molecule which is capable of conferring, activating or enhancing expression of a nucleic acid in a cell. A promoter can comprise one or more specific transcriptional regulatory sequences to further enhance expression and/or to alter the spatial expression and/or temporal expression of same. A promoter can also comprise distal enhancer or repressor elements, which can be located as much as several thousand base pairs from the start site of transcription. A promoter can be derived from sources including viral, bacterial, fungal, plants, insects, and animals. A promoter can regulate the expression of a gene component constitutively or differentially with respect to cell, the tissue or organ in which expression occurs or, with respect to the developmental stage at which expression occurs, or in response to external stimuli such as physiological stresses, pathogens, metal ions, or inducing agents. Representative examples of promoters include the bacteriophage T7 promoter, bacteriophage T3 promoter, SP6 promoter, lac operator-promoter, tac promoter, SV40 late promoter, SV40 early promoter, RSV-LTR promoter, CMV IE promoter, SV40 early promoter or SV40 late promoter and the CMV IE promoter.

“Signal peptide” and “leader sequence” are used interchangeably herein and refer to an amino acid sequence that can be linked at the amino terminus of a IL-17 protein (and/or an antigen) set forth herein. Signal peptides/leader sequences typically direct localization of a protein. Signal peptides/leader sequences used herein preferably facilitate secretion of the protein from the cell in which it is produced. Signal peptides/leader sequences are often cleaved from the remainder of the protein, often referred to as the mature protein, upon secretion from the cell. Signal peptides/leader sequences are linked at the N terminus of the protein.

“Subject” as used herein can mean a mammal that wants to or is in need of being immunized with the herein described vaccine. The mammal can be a human or non-human such as a chimpanzee, dog, cat, horse, cow, pig, chicken, mouse, or rat.

“Substantially identical” as used herein can mean that a first and second amino acid sequence are at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%,or 99% over a region of 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100 or more amino acids. Substantially identical can also mean that a first nucleic acid sequence and a second nucleic acid sequence are at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%,or 99% over a region of 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100 or more nucleotides.

“Treatment” or “treating,” as used herein can mean protecting of an animal from a disease through means of preventing, suppressing, repressing, or completely eliminating the disease. Preventing the disease involves administering a vaccine of the present invention to an animal prior to onset of the disease. Suppressing the disease involves administering a vaccine of the present invention to an animal after induction of the disease but before its clinical appearance. Repressing the disease involves administering a vaccine of the present invention to an animal after clinical appearance of the disease.

“Variant” used herein with respect to a nucleic acid means (i) a portion or fragment of a referenced nucleotide sequence; (ii) the complement of a referenced nucleotide sequence or portion thereof; (iii) a nucleic acid that is substantially identical to a referenced nucleic acid or the complement thereof; or (iv) a nucleic acid that hybridizes under stringent conditions to the referenced nucleic acid, complement thereof, or a sequences substantially identical thereto.

Variant can further be defined as a peptide or polypeptide that differs in amino acid sequence by the insertion, deletion, or conservative substitution of amino acids, but retain at least one biological activity. Representative examples of “biological activity” include the ability to be bound by a specific antibody or to promote an immune response. Variant can also mean a protein with an amino acid sequence that is substantially identical to a referenced protein with an amino acid sequence that retains at least one biological activity. A conservative substitution of an amino acid, i.e., replacing an amino acid with a different amino acid of similar properties (e.g., hydrophilicity, degree and distribution of charged regions) is recognized in the art as typically involving a minor change. These minor changes can be identified, in part, by considering the hydropathic index of amino acids, as understood in the art. Kyte et al., J. Mol. Biol. 157:105-132 (1982). The hydropathic index of an amino acid is based on a consideration of its hydrophobicity and charge. It is known in the art that amino acids of similar hydropathic indexes can be substituted and still retain protein function. In one aspect, amino acids having hydropathic indexes of ±2 are substituted. The hydrophilicity of amino acids can also be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity. Substitution of amino acids having similar hydrophilicity values can result in peptides retaining biological activity, for example immunogenicity, as is understood in the art. Substitutions can be performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

A variant may be a nucleic acid sequence that is substantially identical over the full length of the full gene sequence or a fragment thereof. The nucleic acid sequence may be 80%, 81%, 82%, 83%, 84%, 85%, 86%, 87%, 88%, 89%, 90%, 91%, 92%, 93%, 94%, 95%, 96%, 97%, 98%, 99%, or 100% identical over the full length of the gene sequence or a fragment thereof. A variant may be an amino acid sequence that is substantially identical over the full length of the amino acid sequence or fragment thereof. The amino acid sequence may be 80%, 81%, 82%, 83%, 84%, 85%, 86%, 87%, 88%, 89%, 90%, 91%, 92%, 93%, 94%, 95%, 96%, 97%, 98%, 99%, or 100% identical over the full length of the amino acid sequence or a fragment thereof

“Vector” as used herein means a nucleic acid sequence containing an origin of replication. A vector can be a viral vector, bacteriophage, bacterial artificial chromosome or yeast artificial chromosome. A vector can be a DNA or RNA vector. A vector can be a self-replicating extrachromosomal vector, and preferably, is a DNA plasmid.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

### 2. Vaccine

Provided herein is a vaccine comprising an antigen and an adjuvant. The vaccine can increase antigen presentation and the overall immune response to the antigen in a subject. The combination of antigen and adjuvant induces the immune system more efficiently than a vaccine comprising the antigen alone. This more efficient immune response provides increased efficacy in the treatment and/or prevention of any disease, pathogen, or virus.

The antigen and adjuvant of the vaccine can be administered together or separately to the subject in need thereof. In some instances, IL-17 can be administered separately from the antigen of the vaccine because a greater immune response is elicited in the subject when IL-17 is administered before the antigen as compared to the vaccine comprising the antigen alone. Such separate administration readies the immune system for exposure to the antigen, thereby providing the increased immune response to the antigen in the subject.

In some embodiments, the adjuvant can be administered at least 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 36 hours, 48 hours, 60 hours, 72 hours, 84 hours, or 96 hours before administration of the antigen to the subject. In other embodiments, the adjuvant can be administered at least 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 60 days, or 90 days before administration of the antigen to the subject.

In still other embodiments, the adjuvant can be administered at least 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, or 15 weeks before administration of the antigen to the subject. In other embodiments, the adjuvant can be administered about 12 hours to about 15 weeks, about 12 hours to about 10 weeks, about 12 hours to about 5 weeks, about 12 hours to about 1 week, about 12 hours to about 60 hours, about 12 hours to about 48 hours, about 24 hours to about 15 weeks, about 60 hours to about 15 weeks, about 96 hours to about 15 weeks, about 1 day to about 15 weeks, about 5 days to about 15 weeks, about 10 days to about 15 weeks, about 15 days to about 15 weeks, about 20 days to about 15 weeks, about 25 days to about 15 weeks, about 30 days to about 15 weeks, about 1 week to about 15 weeks, about 5 weeks to about 15 weeks, or about 10 weeks to about 15 weeks before administration of the antigen to the subject.

In another embodiment, the antigen and the IL-17 are administered simultaneously. In such a case, they may be formulated together (e.g., in admixture in a same container) or separately (and administered at a same time).

In alternative embodiments, the IL-17 may be administered alone first, and then again, in admixture with or at the same time as the antigen

The vaccine of the present invention can have features required of effective vaccines such as being safe so the vaccine itself does not cause illness or death; being protective against illness resulting from exposure to live pathogens such as viruses or bacteria; inducing neutralizing antibody to prevent infection of cells; inducing protective T cell against intracellular pathogens; and providing ease of administration, few side effects, biological stability, and low cost per dose. The vaccine can accomplish some or all of these features by combining the antigen with the adjuvant as discussed below.

The vaccine can further modify epitope presentation within the antigen to induce greater immune response to the antigen that a vaccine comprising the antigen alone. The vaccine can further induce an immune response when administered to different tissues such as the muscle or the skin.

### a. IL-17

The present invention uses IL-17, in vaccines or compositions, to induce or stimulate an immune response in a subject. The IL-17 can be a nucleic acid sequence, an amino acid sequence, or a combination thereof. The nucleic acid sequence can be DNA, RNA, cDNA, a variant thereof, a fragment thereof, or a combination thereof. The nucleic acid sequence can also include additional sequence that encode e.g., linker or tag sequence, that is linked to the IL-17 sequence by a peptide bond. The amino acid sequence can be a protein, a peptide, a variant thereof, a fragment thereof, or a combination thereof.

The term IL-17 designates interleukin-17, a monomer thereof, a dimer thereof, a homodimer thereof, a heterodimer thereof, a fragment thereof, a variant thereof, or a combination thereof. The IL-17 may be IL-17A (also known as CTLA-8), IL-17B, IL-17C, IL-17D, IL-17E (also known as IL-25) and IL-17F. IL-17 may be isolated and originate from multiple cell types, for example, T helper type 17 (Th17) cells, activated T cells, and epithelial cells. IL-17 cytokines have a highly conserved C-terminus, which contains a cysteine-knot fold structure, and are secreted as disulfide-linked dimers with the exception of IL-17B. IL-17B is secreted as a non-covalent dimer. Besides homodimers, heterodimers can be formed between IL-17A and IL-17F.

The vaccine can comprise about 0.001 μg to about 100 μg of IL-17, preferably from about 0.001 μg to about 50 μg, more preferably from about 0.001 μg to about 10 μg, even more preferably from about 0.001 μg to about 5 μg, about 0.005 μg to about 4 μg, about 0.01 μg to about 3 μg, about 0.05 μg to about 2 μg, or about 0.1 μg to about 1 μg of IL-17. The immunotherapeutic composition can comprise at least about 0.001 μg, at least about 0.002 μg, at least about 0.003 μg, at least about 0.004 μg, at least about 0.005 μg, at least about 0.006 μg, at least about 0.007 μg, at least about 0.008 μg, at least about 0.009 μg, at least about 0.01 μg, at least about 0.02 μg, at least about 0.03 μg, at least about 0.04 μg, at least about 0.05 μg, at least about 0.06 μg, at least about 0.07 μg, at least about 0.08 μg, at least about 0.09 μg, at least about 0.1 μg, at least about 0.2 μg, at least about 0.3 μg, at least about 0.4 μg, at least about 0.5 μg, at least about 0.6 μg, at least about 0.7 μg, at least about 0.8 μg, at least about 0.9 μg, at least about 1.0 μg, at least about 1.5 μg, at least about 2.0 μg, at least about 2.5 μg, at least about 3.0 μg, at least about 3.5 μg, at least about 4.0 μg, at least about 4.5 μg, or at least about 5.0 μg of IL-17.

Inclusion of IL-17 in the vaccine can increase or boost the immune response to the antigen in the subject as compared to a vaccine not including IL-17. The antigen is discussed in more detail below. In some instances, inclusion of IL-17 in the vaccine can increase the immune response to the antigen by about 0.5-fold to about 15-fold, about 0.5-fold to about 10-fold, or about 0.5-fold to about 8-fold as compared to the vaccine not including IL-17. Alternatively, inclusion of IL-17 in the vaccine can increase the immune response to the antigen by at least about 0.5-fold, at least about 1.0-fold, at least about 1.5-fold, at least about 2.0-fold, at least about 2.5-fold, at least about 3.0-fold, at least about 3.5-fold, at least about 4.0-fold, at least about 4.5-fold, at least about 5.0-fold, at least about 5.5-fold, at least about 6.0-fold, at least about 6.5-fold, at least about 7.0-fold, at least about 7.5-fold, at least about 8.0-fold, at least about 8.5-fold, at least about 9.0-fold, at least about 9.5-fold, at least about 10.0-fold, at least about 10.5-fold, at least about 11.0-fold, at least about 11.5-fold, at least about 12.0-fold, at least about 12.5-fold, at least about 13.0-fold, at least about 13.5-fold, at least about 14.0-fold, at least about 14.5-fold, or at least about 15.0-fold as compared to the vaccine not including IL-17.

In still other alternative embodiments, inclusion of IL-17 in the vaccine can increase the immune response to the antigen by about 50% to about 1500%, about 50% to about 1000%, or about 50% to about 800% as compared to the vaccine not including IL-17. In other embodiments, inclusion of IL-17 in the vaccine can increase the immune response to the antigen by at least about 50%, at least about 100%, at least about 150%, at least about 200%, at least about 250%, at least about 300%, at least about 350%, at least about 400%, at least about 450%, at least about 500%, at least about 550%, at least about 600%, at least about 650%, at least about 700%, at least about 750%, at least about 800%, at least about 850%, at least about 900%, at least about 950%, at least about 1000%, at least about 1050%, at least about 1100%, at least about 1150%, at least about 1200%, at least about 1250%, at least about 1300%, at least about 1350%, at least about 1450%, or at least about 1500% as compared to the vaccine not including IL-17.

The IL-17 cytokine can signal through members of the IL-17 receptor family and activation of these receptors triggers intercellular pathways that induce the production of pro-inflammatory cytokines, for example, interferon-gamma (IFN-γ), IL-6, and tumor necrosis factor alpha (TNF-α). Accordingly, overexpression of the IL-17 cytokines can contribute to the development of autoimmune and inflammatory diseases such as asthma, rheumatoid arthritis, psoriasis, transplant rejection, inflammatory bowel disease, and multiple sclerosis.

IFN-γ has antiviral, immunoregulatory, and anti-tumor properties and can alter transcription in multiple genes to produce a variety of physiological and cellular responses. Some effects by IFN-γ include promoting natural killer (NK) cell activity, causing normal cells to increase expression of class I MHC molecules, increasing antigen presentation and lysosome activity in macrophages, inducing nitric oxide synthase (iNOS), and promoting Th1 differentiation to cellular immunity regarding cytotoxic CD8⁺ T cells while suppressing Th2 differentiation in humoral (antibody) response.

Cytotoxic CD8⁺ T cells (cytotoxic T lymphocytes (CTLs)) are a subgroup of T cells that induce the death of cells infected with viruses and other pathogens. Upon activation, CTLs undergo clonal expansion to produce effector cells that are antigen-specific. Effector CTLs release through a process of directed exocytosis (i.e., degranulation) molecules that kill infected or target cells, for example, perforin, granulysin, and granzyme. When no longer needed, many effector CTLs die, but some effector cells are retained as memory cells such that when the antigen is encountered again, the memory cells differentiate into effector cells to more quickly mount an immune response.

When IL-17 increases or boosts the immune response, this increased immune response can include an increased cellular immune response. The increased cellular immune response can include an increased CD8⁺ T cell response. The increased CD8⁺ T cell response can include an increased cytotoxic CD8⁺ T lymphocyte (CTL) response. The increased CTL response can include an increased amount of antigen-specific-lysis of target cells. The increased CD8⁺ T cell response can include increasing the population or frequency of CD8⁺ T cells secreting IFN-γ. The IFN-γ producing CD8⁺ T cells can have cytolytic activity.

In turn, inclusion of IL-17 in the vaccine can increase the level of IFN-γ by about 0.5-fold to about 15-fold, about 0.5-fold to about 10-fold, or about 0.5-fold to about 8-fold as compared to the vaccine not including IL-17. Inclusion of IL-17 in the vaccine can increase the level of IFN-γ by at least about 0.5-fold, at least about 1.0-fold, at least about 1.5-fold, at least about 2.0-fold, at least about 2.5-fold, at least about 3.0-fold, at least about 3.5-fold, at least about 4.0-fold, at least about 4.5-fold, at least about 5.0-fold, at least about 5.5-fold, at least about 6.0-fold, at least about 6.5-fold, at least about 7.0-fold, at least about 7.5-fold, at least about 8.0-fold, at least about 8.5-fold, at least about 9.0-fold, at least about 9.5-fold, at least about 10.0-fold, at least about 10.5-fold, at least about 11.0-fold, at least about 11.5-fold, at least about 12.0-fold, at least about 12.5-fold, at least about 13.0-fold, at least about 13.5-fold, at least about 14.0-fold, at least about 14.5-fold, or at least about 15.0-fold as compared to the vaccine not including IL-17.

Inclusion of IL-17 in the vaccine can also increase the level of IFN-γ by about 50% to about 1500%, about 50% to about 1000%, or about 50% to about 800% as compared to the vaccine not including IL-17. Inclusion of IL-17 in the vaccine can also increase the level of IFN-γ by at least about 50%, at least about 100%, at least about 150%, at least about 200%, at least about 250%, at least about 300%, at least about 350%, at least about 400%, at least about 450%, at least about 500%, at least about 550%, at least about 600%, at least about 650%, at least about 700%, at least about 750%, at least about 800%, at least about 850%, at least about 900%, at least about 950%, at least about 1000%, at least about 1050%, at least about 1100%, at least about 1150%, at least about 1200%, at least about 1250%, at least about 1300%, at least about 1350%, at least about 1450%, or at least about 1500% as compared to the vaccine not including IL-17.

When IL-17 increases or boosts the immune response, this increased immune response can include an increased humoral immune response. This increased humoral immune response can include increased antibody titers and increased duration of the antibody response. Accordingly, inclusion of IL-17 in the vaccine can increase the humoral immune response as compared to the vaccine not including IL-17. Inclusion of IL-17 in the vaccine can increase the antibody titer specific for the antigen as compared to the vaccine not including IL-17. Inclusion of IL-17 in the vaccine can increase the duration of the antibody response to the antigen as compared to the vaccine not including IL-17.

An object of the invention thus relates to an IL-17 protein or nucleic acid, for use as an adjuvant.

The invention also relates to an IL-17 protein or nucleic acid, for use to stimulate an antigen-specific immune response in a subject.

The invention also concerns an IL-17 protein or nucleic acid, for use to induce or stimulate a CD8-T cell response to an antigen in a subject.

The invention also relates to an IL-17 protein or nucleic acid, in combination with an antigen, for use to vaccinate a subject.

In a preferred embodiment, IL-17 is IL-17A, a fragment thereof, a variant thereof, or a combination thereof. IL-17A can be a monomer, a homodimer, or a heterodimer with IL-17F.

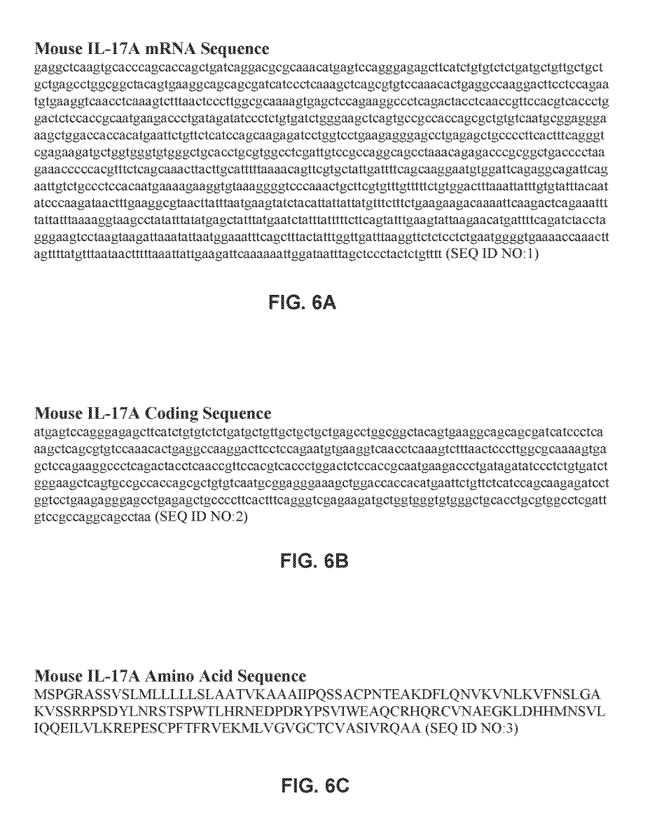

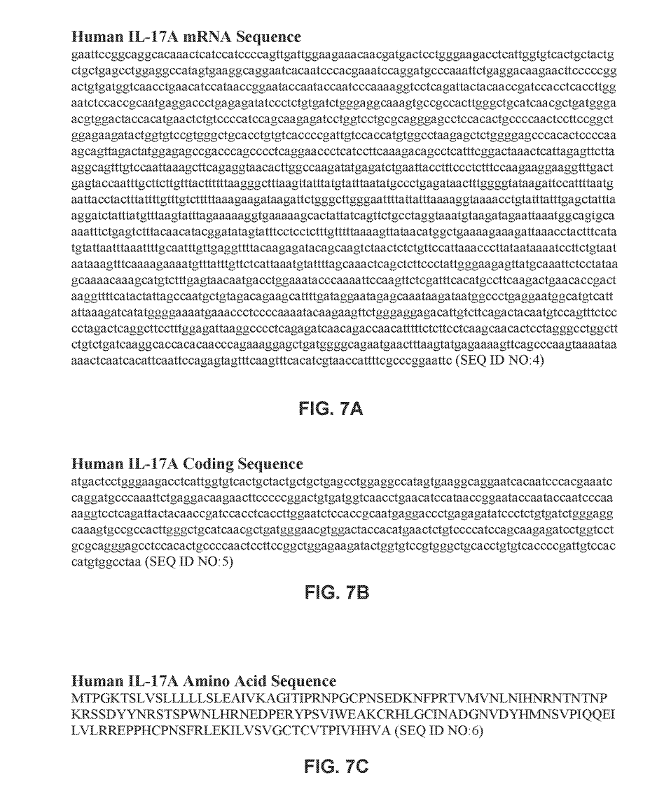

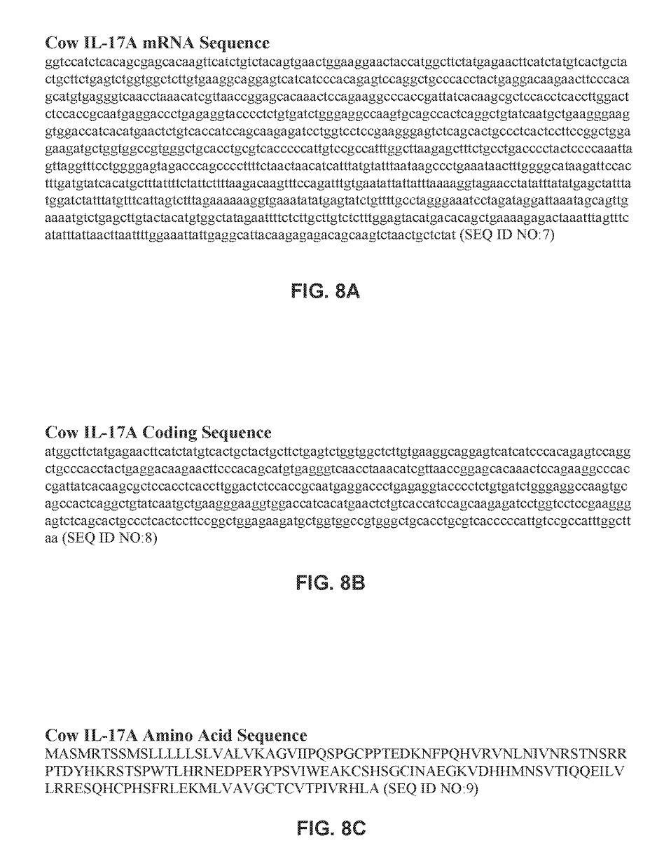

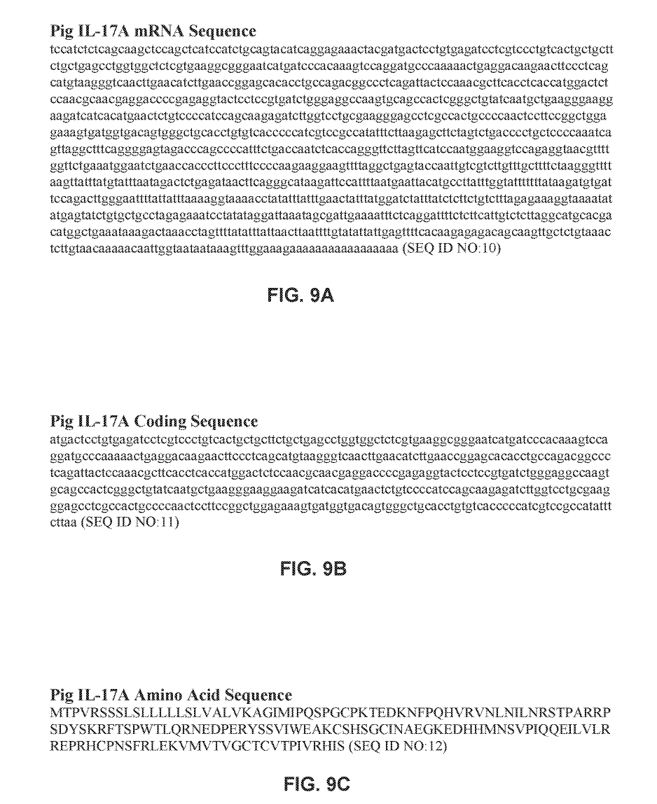

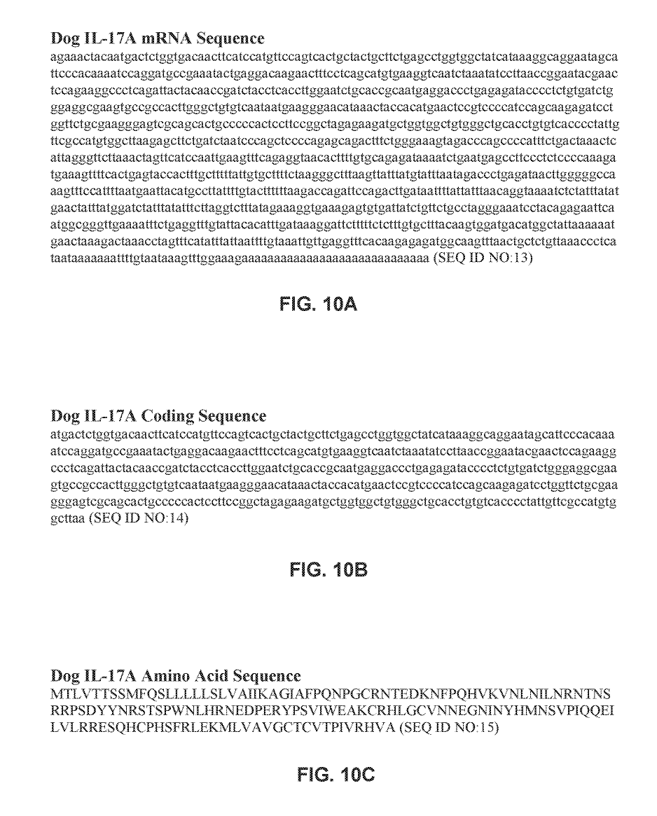

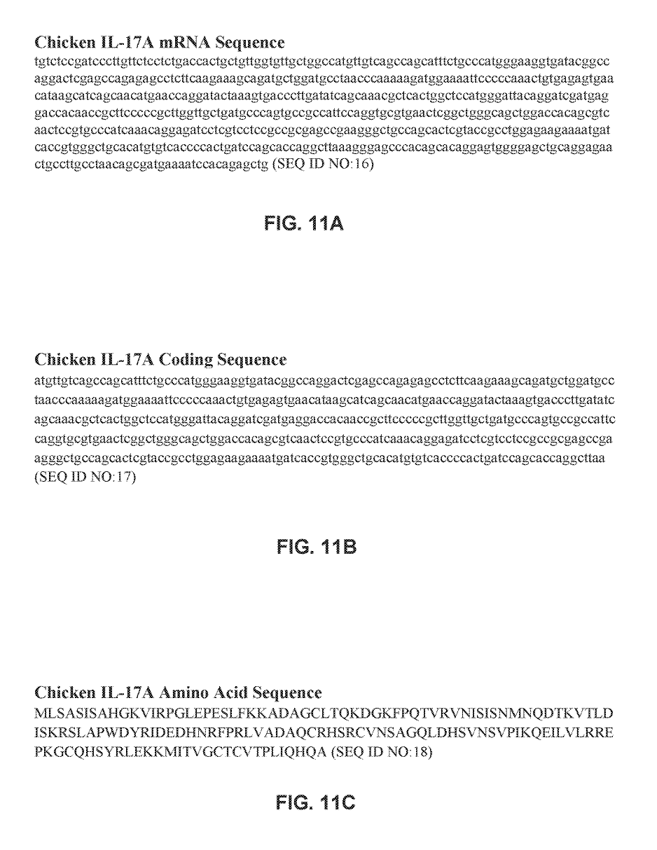

IL-17A can be an IL-17A protein from any number of organisms, for example, a mouse (Mus musculus) IL-17A protein, a human (Homo sapiens) IL-17A protein, a cow (Bos taurus) IL-17A protein, a pig (Sus scrofa) IL-17A protein, a dog (Canis lupis familiaris) IL-17A protein, and chicken (Gallus gallus) IL-17A protein. The mouse IL-17A protein can have the amino acid sequence shown in FIG. 6C (i.e., SEQ ID NO:3), a fragment thereof, a variant thereof, or a combination thereof. The human IL-17A protein can have the amino acid sequence shown in FIG. 7C (i.e., SEQ ID NO:6), a fragment thereof, a variant thereof, or a combination thereof. The cow IL-17A protein can have the amino acid sequence shown in FIG. 8C (i.e., SEQ ID NO:9), a fragment thereof, a variant thereof, or a combination thereof. The pig IL-17A protein can have the amino acid sequence shown in FIG. 9C (i.e., SEQ ID NO:12), a fragment thereof, a variant thereof, or a combination thereof. The dog IL-17A protein can have the amino acid sequence shown in FIG. 10C (i.e., SEQ ID NO:15), a fragment thereof, a variant thereof, or a combination thereof. The chicken IL-17A protein can have the amino acid sequence shown in FIG. 11C (SEQ ID NO:18), a fragment thereof, a variant thereof, or a combination thereof. The IL-17A protein may further comprise one or more additional amino acid sequence elements, for example, an immunoglobulin E (IgE) leader sequence (e.g., SEQ ID NO:38), an hemagglutinin (HA) tag, or both an IgE leader sequence and an HA tag.

A nucleic acid encoding IL-17A can be from any number of organisms, for example, mouse (Mus musculus) (FIGS. 6A and 6B, SEQ ID NOS:1 and 2, respectively), human (Homo sapiens) (FIGS. 7A and 7B, SEQ ID NOS:4 and 5, respectively), cow (Bos taurus) (FIGS. 8A and 8B, SEQ ID NOS:7 and 8, respectively), pig (Sus scrofa) (FIGS. 9A and 9B, SEQ ID NOS:10 and 11, respectively), dog (Canis lupis familiaris) (FIGS. 10A and 10B, SEQ ID NOS:13 and 14, respectively), and chicken (Gallus gallus) (FIGS. 11A and 11B, SEQ ID NOS:16 and 17, respectively). The nucleic acid encoding IL-17A can be optimized with regards to codon usage and corresponding RNA transcripts. The nucleic acid encoding IL-17A can be codon and RNA optimized for expression. The nucleic acid encoding IL-17A can include a Kozak sequence (e.g., GCC ACC) to increase the efficiency of translational initiation. The nucleic acid encoding IL-17A can include multiple stop codons (e.g., TGA TAA) to increase the efficiency of translational termination. The nucleic acid encoding IL-17A can also encode an immunoglobulin E (IgE) leader sequence. The IgE leader sequence can be located 5′ to IL-17A in the nucleic acid. The nucleic acid encoding IL-17A can also include a nucleotide sequence encoding the IgE leader sequence (e.g., SEQ ID NO:37).

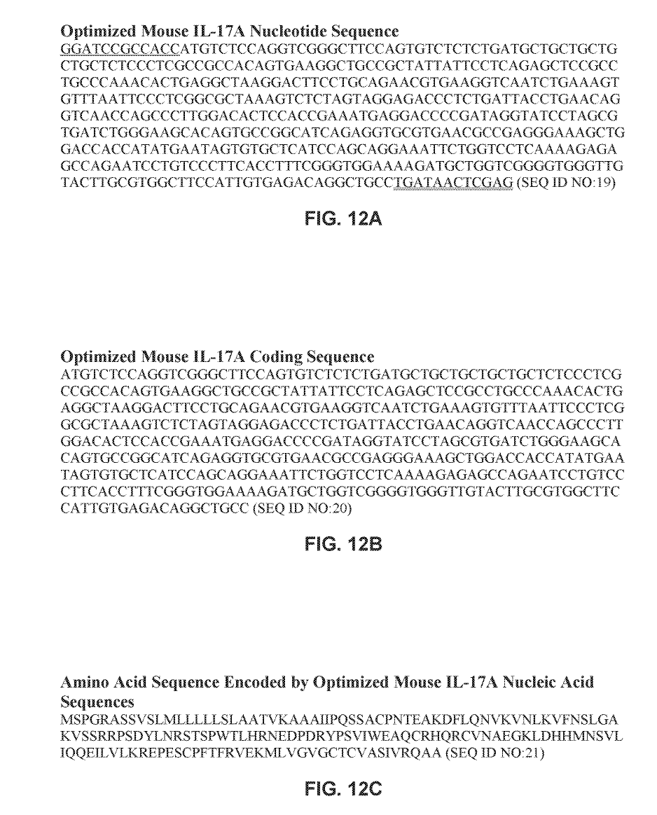

The optimized mouse IL-17A can be the nucleic acid sequence SEQ ID NO:19, which encodes SEQ ID NO:21 (FIGS. 12A and 12C). In some embodiments, the optimized mouse IL-17A can be the nucleic acid sequence having at least about 80%, 81%, 82%, 83%, 84%, 85%, 86%, 87%, 88%, 89%, 90%, 91%, 92%, 93%, 94%, 95%, 96%, 97%, 98%, 99%, or 100% identity over an entire length of the nucleic acid sequence set forth in SEQ ID NO:19. In other embodiments, the optimized mouse IL-17A can be the nucleic acid sequence that encodes the amino acid sequence having at least about 80%, 81%, 82%, 83%, 84%, 85%, 86%, 87%, 88%, 89%, 90%, 91%, 92%, 93%, 94%, 95%, 96%, 97%, 98%, 99%, or 100% identity over an entire length of the amino acid sequence set forth in SEQ ID NO:21.

The optimized mouse IL-17A can be the nucleic acid sequence SEQ ID NO:20, which encodes SEQ ID NO:21 (FIGS. 12B and 12C). In some embodiments, the optimized mouse IL-17A can be the nucleic acid sequence having at least about 80%, 81%, 82%, 83%, 84%, 85%, 86%, 87%, 88%, 89%, 90%, 91%, 92%, 93%, 94%, 95%, 96%, 97%, 98%, 99%, or 100% identity over an entire length of the nucleic acid sequence set forth in SEQ ID NO:20.

The mouse IL-17A can be the amino acid sequence having at least about 80%, 81%, 82%, 83%, 84%, 85%, 86%, 87%, 88%, 89%, 90%, 91%, 92%, 93%, 94%, 95%, 96%, 97%, 98%, 99%, or 100% identity over an entire length of the amino acid sequence set forth in SEQ ID NO:21.

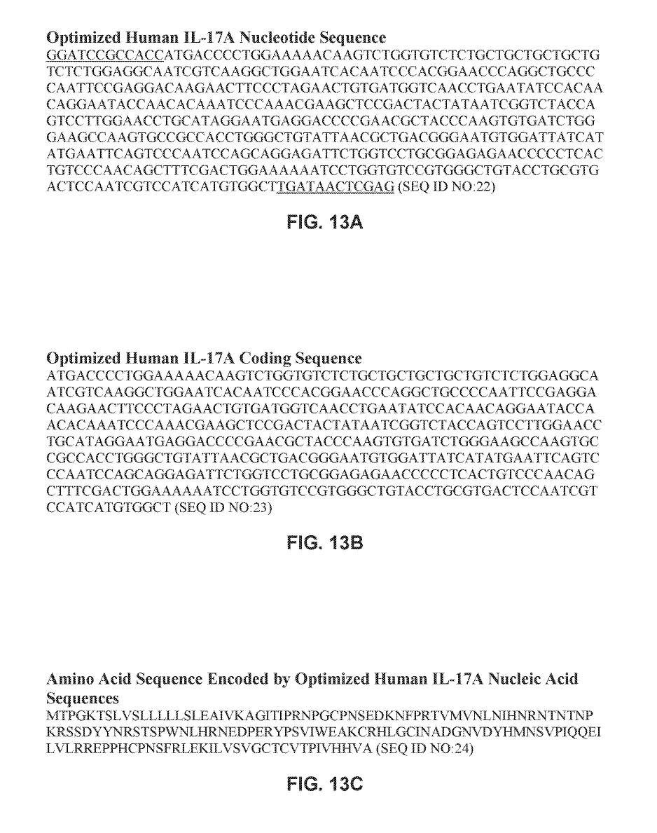

The optimized human IL-17A can be the nucleic acid sequence SEQ ID NO:22, which encodes SEQ ID NO:24 (FIGS. 13A and 13C). In some embodiments, the optimized human IL-17A can be the nucleic acid sequence having at least about 80%, 81%, 82%, 83%, 84%, 85%, 86%, 87%, 88%, 89%, 90%, 91%, 92%, 93%, 94%, 95%, 96%, 97%, 98%, 99%, or 100% identity over an entire length of the nucleic acid sequence set forth in SEQ ID NO:22. In other embodiments, the optimized human IL-17A can be the nucleic acid sequence that encodes the amino acid sequence having at least about 80%, 81%, 82%, 83%, 84%, 85%, 86%, 87%, 88%, 89%, 90%, 91%, 92%, 93%, 94%, 95%, 96%, 97%, 98%, 99%, or 100% identity over an entire length of the amino acid sequence set forth in SEQ ID NO:24.

The optimized human IL-17A can be the nucleic acid sequence SEQ ID NO:23, which encodes SEQ ID NO:24 (FIGS. 13B and 13C). In some embodiments, the optimized human IL-17A can be the nucleic acid sequence having at least about 80%, 81%, 82%, 83%, 84%, 85%, 86%, 87%, 88%, 89%, 90%, 91%, 92%, 93%, 94%, 95%, 96%, 97%, 98%, 99%, or 100% identity over an entire length of the nucleic acid sequence set forth in SEQ ID NO:23.

The human IL-17A can be the amino acid sequence having at least about 80%, 81%, 82%, 83%, 84%, 85%, 86%, 87%, 88%, 89%, 90%, 91%, 92%, 93%, 94%, 95%, 96%, 97%, 98%, 99%, or 100% identity over an entire length of the amino acid sequence set forth in SEQ ID NO:24.

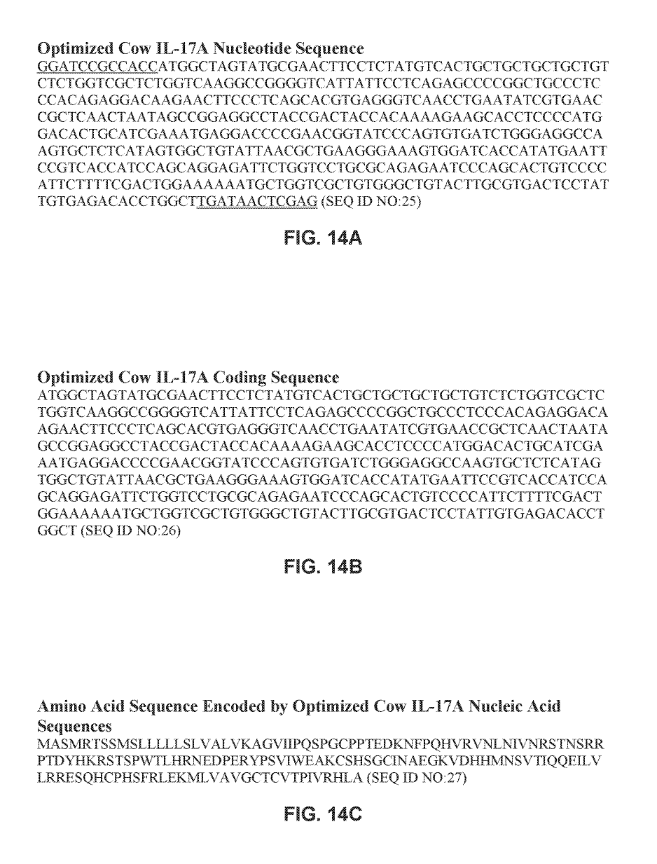

The optimized cow IL-17A can be the nucleic acid sequence SEQ ID NO:25, which encodes SEQ ID NO:27 (FIGS. 14A and 14C). In some embodiments, the optimized cow IL-17A can be the nucleic acid sequence having at least about 80%, 81%, 82%, 83%, 84%, 85%, 86%, 87%, 88%, 89%, 90%, 91%, 92%, 93%, 94%, 95%, 96%, 97%, 98%, 99%, or 100% identity over an entire length of the nucleic acid sequence set forth in SEQ ID NO:25. In other embodiments, the optimized cow IL-17A can be the nucleic acid sequence that encodes the amino acid sequence having at least about 80%, 81%, 82%, 83%, 84%, 85%, 86%, 87%, 88%, 89%, 90%, 91%, 92%, 93%, 94%, 95%, 96%, 97%, 98%, 99%, or 100% identity over an entire length of the amino acid sequence set forth in SEQ ID NO:27.

The optimized cow IL-17A can be the nucleic acid sequence SEQ ID NO:26, which encodes SEQ ID NO:27 (FIGS. 14B and 14C). In some embodiments, the optimized cow IL-17A can be the nucleic acid sequence having at least about 80%, 81%, 82%, 83%, 84%, 85%, 86%, 87%, 88%, 89%, 90%, 91%, 92%, 93%, 94%, 95%, 96%, 97%, 98%, 99%, or 100% identity over an entire length of the nucleic acid sequence set forth in SEQ ID NO:26.

The cow IL-17A can be the amino acid sequence having at least about 80%, 81%, 82%, 83%, 84%, 85%, 86%, 87%, 88%, 89%, 90%, 91%, 92%, 93%, 94%, 95%, 96%, 97%, 98%, 99%, or 100% identity over an entire length of the amino acid sequence set forth in SEQ ID NO:27.

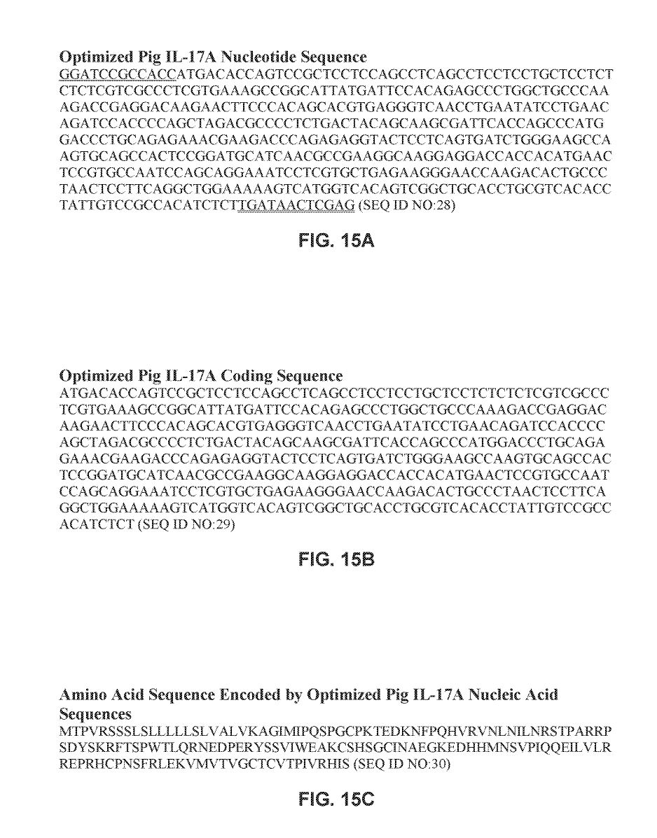

The optimized pig IL-17A can be the nucleic acid sequence SEQ ID NO:28, which encodes SEQ ID NO:30 (FIGS. 15A and 15C). In some embodiments, the optimized pig IL-17A can be the nucleic acid sequence having at least about 80%, 81%, 82%, 83%, 84%, 85%, 86%, 87%, 88%, 89%, 90%, 91%, 92%, 93%, 94%, 95%, 96%, 97%, 98%, 99%, or 100% identity over an entire length of the nucleic acid sequence set forth in SEQ ID NO:28. In other embodiments, the optimized pig IL-17A can be the nucleic acid sequence that encodes the amino acid sequence having at least about 80%, 81%, 82%, 83%, 84%, 85%, 86%, 87%, 88%, 89%, 90%, 91%, 92%, 93%, 94%, 95%, 96%, 97%, 98%, 99%, or 100% identity over an entire length of the amino acid sequence set forth in SEQ ID NO:30.

The optimized pig IL-17A can be the nucleic acid sequence SEQ ID NO:29, which encodes SEQ ID NO:30 (FIGS. 15B and 15C). In some embodiments, the optimized pig IL-17A can be the nucleic acid sequence having at least about 80%, 81%, 82%, 83%, 84%, 85%, 86%, 87%, 88%, 89%, 90%, 91%, 92%, 93%, 94%, 95%, 96%, 97%, 98%, 99%, or 100% identity over an entire length of the nucleic acid sequence set forth in SEQ ID NO:29.

The pig IL-17A can be the amino acid sequence having at least about 80%, 81%, 82%, 83%, 84%, 85%, 86%, 87%, 88%, 89%, 90%, 91%, 92%, 93%, 94%, 95%, 96%, 97%, 98%, 99%, or 100% identity over an entire length of the amino acid sequence set forth in SEQ ID NO:30.

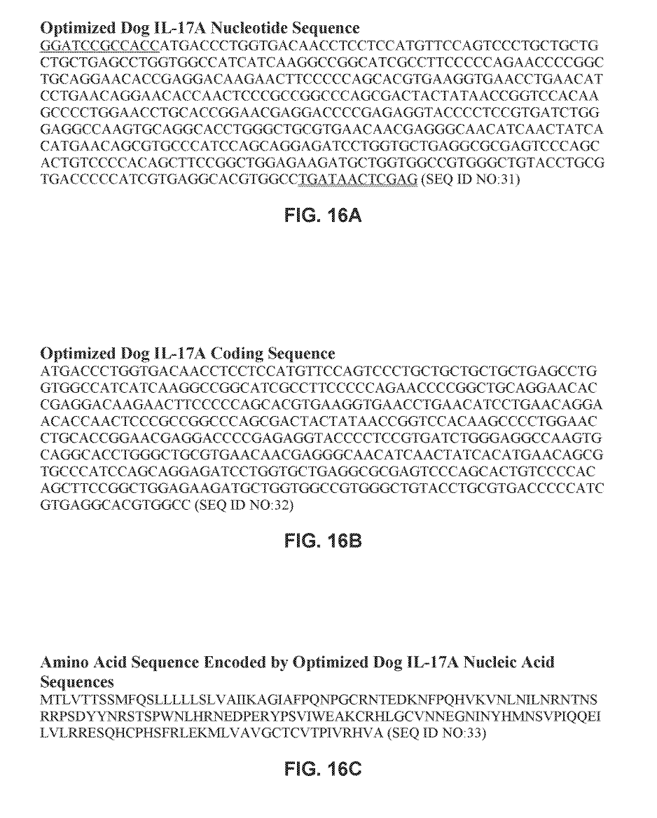

The optimized dog IL-17A can be the nucleic acid sequence SEQ ID NO:31, which encodes SEQ ID NO:33 (FIGS. 16A and 16C). In some embodiments, the optimized dog IL-17A can be the nucleic acid sequence having at least about 80%, 81%, 82%, 83%, 84%, 85%, 86%, 87%, 88%, 89%, 90%, 91%, 92%, 93%, 94%, 95%, 96%, 97%, 98%, 99%, or 100% identity over an entire length of the nucleic acid sequence set forth in SEQ ID NO:31. In other embodiments, the optimized dog IL-17A can be the nucleic acid sequence that encodes the amino acid sequence having at least about 80%, 81%, 82%, 83%, 84%, 85%, 86%, 87%, 88%, 89%, 90%, 91%, 92%, 93%, 94%, 95%, 96%, 97%, 98%, 99%, or 100% identity over an entire length of the amino acid sequence set forth in SEQ ID NO:33.

The optimized dog IL-17A can be the nucleic acid sequence SEQ ID NO:32, which encodes SEQ ID NO:33 (FIGS. 16B and 16C). In some embodiments, the optimized dog IL-17A can be the nucleic acid sequence having at least about 80%, 81%, 82%, 83%, 84%, 85%, 86%, 87%, 88%, 89%, 90%, 91%, 92%, 93%, 94%, 95%, 96%, 97%, 98%, 99%, or 100% identity over an entire length of the nucleic acid sequence set forth in SEQ ID NO:32.

The dog IL-17A can be the amino acid sequence having at least about 80%, 81%, 82%, 83%, 84%, 85%, 86%, 87%, 88%, 89%, 90%, 91%, 92%, 93%, 94%, 95%, 96%, 97%, 98%, 99%, or 100% identity over an entire length of the amino acid sequence set forth in SEQ ID NO:33.

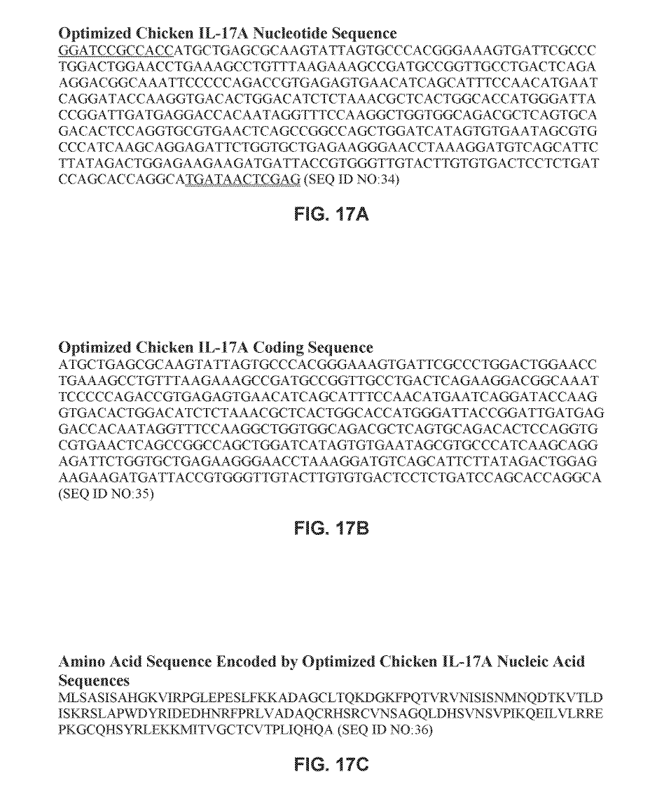

The optimized chicken IL-17A can be the nucleic acid sequence SEQ ID NO:34, which encodes SEQ ID NO:36 (FIGS. 17A and 17C). In some embodiments, the optimized chicken IL-17A can be the nucleic acid sequence having at least about 80%, 81%, 82%, 83%, 84%, 85%, 86%, 87%, 88%, 89%, 90%, 91%, 92%, 93%, 94%, 95%, 96%, 97%, 98%, 99%, or 100% identity over an entire length of the nucleic acid sequence set forth in SEQ ID NO:34. In other embodiments, the optimized chicken IL-17A can be the nucleic acid sequence that encodes the amino acid sequence having at least about 80%, 81%, 82%, 83%, 84%, 85%, 86%, 87%, 88%, 89%, 90%, 91%, 92%, 93%, 94%, 95%, 96%, 97%, 98%, 99%, or 100% identity over an entire length of the amino acid sequence set forth in SEQ ID NO:36.

The optimized chicken IL-17A can be the nucleic acid sequence SEQ ID NO:35, which encodes SEQ ID NO:36 (FIGS. 17B and 17C). In some embodiments, the optimized chicken IL-17A can be the nucleic acid sequence having at least about 80%, 81%, 82%, 83%, 84%, 85%, 86%, 87%, 88%, 89%, 90%, 91%, 92%, 93%, 94%, 95%, 96%, 97%, 98%, 99%, or 100% identity over an entire length of the nucleic acid sequence set forth in SEQ ID NO:35.

The chicken IL-17A can be the amino acid sequence having at least about 80%, 81%, 82%, 83%, 84%, 85%, 86%, 87%, 88%, 89%, 90%, 91%, 92%, 93%, 94%, 95%, 96%, 97%, 98%, 99%, or 100% identity over an entire length of the amino acid sequence set forth in SEQ ID NO:36.

Immunogenic fragments of SEQ ID NO:21, SEQ ID NO:24, SEQ ID NO:27, SEQ ID NO:30, SEQ ID NO:33, and SEQ ID NO:36 can be provided. Immunogenic fragments can comprise at least 60%, at least 65%, at least 70%, at least 75%, at least 80%, at least 85%, at least 90%, at least 95%, at least 96%, at least 97%, at least 98%, or at least 99% of SEQ ID NO:21, SEQ ID NO:24, SEQ ID NO:27, SEQ ID NO:30, SEQ ID NO:33, and/or SEQ ID NO:36. In some embodiments, immunogenic fragments include a leader sequence, for example, an immunoglobulin leader sequence, such as the immunoglobulin E (IgE) leader sequence. In some embodiments, immunogenic fragments are free of a leader sequence.

Immunogenic fragments of proteins with amino acid sequences having identity to immunogenic fragments of SEQ ID NO:21, SEQ ID NO:24, SEQ ID NO:27, SEQ ID NO:30, SEQ ID NO:33, and SEQ ID NO:36 can be provided. Such fragments can comprise at least 60%, at least 65%, at least 70%, at least 75%, at least 75%, at least 80%, at least 85%, at least 90%, at least 95%, at least 96%, at least 97%, at least 98%, or at least 99% of proteins having 95% or greater identity to SEQ ID NO:21, SEQ ID NO:24, SEQ ID NO:27, SEQ ID NO:30, SEQ ID NO:33, and/or SEQ ID NO:36. Some embodiments relate to immunogenic fragments that have 96% or greater identity to the immunogenic fragments of IL-17A protein sequences herein. Some embodiments relate to immunogenic fragments that have 97% or greater identity to the immunogenic fragments of IL-17A protein sequences herein. Some embodiments relate to immunogenic fragments that have 98% or greater identity to the immunogenic fragments of IL-17A protein sequences herein. Some embodiments relate to immunogenic fragments that have 99% or greater identity to the immunogenic fragments of IL-17A protein sequences herein. In some embodiments, immunogenic fragments include a leader sequence, for example, an immunoglobulin leader sequence such as the IgE leader sequence. In some embodiments, the immunogenic fragments are free of a leader sequence.

Some embodiments relate to immunogenic fragments of SEQ ID NO:19, SEQ ID NO:20, SEQ ID NO:22, SEQ ID NO::23, SEQ ID NO:25, SEQ ID NO:26, SEQ ID NO:28, SEQ ID NO:29, SEQ ID NO:31, SEQ ID NO:32, SEQ ID NO:34, and SEQ ID NO:35. Immunogenic fragments can comprise at least 60%, at least 65%, at least 70%, at least 75%, at least 80%, at least 85%, at least 90%, at least 95%, at least 96%, at least 97%, at least 98%, or at least 99% of SEQ ID NO:19, SEQ ID NO:20, SEQ ID NO:22, SEQ ID NO::23, SEQ ID NO:25, SEQ ID NO:26, SEQ ID NO:28, SEQ ID NO:29, SEQ ID NO:31, SEQ ID NO:32, SEQ ID NO:34, and/or SEQ ID NO:35. In some embodiments, immunogenic fragments include sequences that encode a leader sequence, for example, an immunoglobulin leader sequence such as the IgE leader sequence. In some embodiments, immunogenic fragments are free of coding sequences that encode a leader sequence.

Immunogenic fragments of nucleic acids with nucleotide sequences having identity to immunogenic fragments of SEQ ID NO:19, SEQ ID NO:20, SEQ ID NO:22, SEQ ID NO::23, SEQ ID NO:25, SEQ ID NO:26, SEQ ID NO:28, SEQ ID NO:29, SEQ ID NO:31, SEQ ID NO:32, SEQ ID NO:34, and SEQ ID NO:35 can be provided. Such fragments can comprise at least 60%, at least 65%, at least 70%, at least 75%, at least 75%, at least 80%, at least 85%, at least 90%, at least 95%, at least 96%, at least 97%, at least 98%, or at least 99% of nucleic acids having 95% or greater identity to SEQ ID NO:19, SEQ ID NO:20, SEQ ID NO:22, SEQ ID NO::23, SEQ ID NO:25, SEQ ID NO:26, SEQ ID NO:28, SEQ ID NO:29, SEQ ID NO:31, SEQ ID NO:32, SEQ ID NO:34, and/or SEQ ID NO:35. Some embodiments relate to immunogenic fragments that have 96% or greater identity to the immunogenic fragments of IL-17A nucleic acid sequences herein. Some embodiments relate to immunogenic fragments that have 97% or greater identity to the immunogenic fragments of IL-17A nucleic acid sequences herein. Some embodiments relate to immunogenic fragments that have 98% or greater identity to the immunogenic fragments of IL-17A nucleic acid sequences herein. Some embodiments relate to immunogenic fragments that have 99% or greater identity to the immunogenic fragments of IL-17A nucleic sequences herein. In some embodiments, immunogenic fragments include sequences that encode a leader sequence, for example, an immunoglobulin leader sequence such as the IgE leader sequence. In some embodiments, immunogenic fragments are free of coding sequences that encode a leader sequence.

### b. Antigen

The antigen can be anything that induces an immune response in a subject. Purified antigens are not usually strong immunogenic on their own and are therefore combined with the adjuvant as described above. The immune response to an antigen can be induced or boosted or increased when combined with the adjuvant. Such an immune response can be a humoral immune response and/or a cellular immune response. In some embodiments, the combination of the adjuvant and the antigen can boost or increase a cellular immune response in the subject.

The antigen can be a nucleic acid sequence, an amino acid sequence, a lipid, a polysaccharide, or a combination thereof. The nucleic acid sequence can be DNA, RNA, cDNA, a variant thereof, a fragment thereof, or a combination thereof. The nucleic acid sequence can also include additional sequences that encode linker or tag sequences that are linked to the antigen by a peptide bond. The amino acid sequence can be a protein, a peptide, a variant thereof, a fragment thereof, or a combination thereof. The antigen may be in purified form, or a mixture such as an extract, a cell, a viral particle, etc.

In a particular embodiment, the antigen is or comprises a protein, a nucleic acid, or a fragment thereof, or a variant thereof, or a combination thereof from any number of organisms, for example, a virus, a parasite, a bacterium, a fungus, or a mammal.

The antigen can be associated with an infectious disease (e.g., a viral disease or a bacterial disease or a parasitic disease), an autoimmune disease, a cancer, allergy, or asthma. In particular embodiments, the antigen can be associated with herpes, influenza, hepatitis B, hepatitis C, human papilloma virus (HPV), or human immunodeficiency virus (HIV). Preferably, the antigen can be associated with influenza or HIV.

Some antigens can induce a strong immune response. Other antigens can induce a weak immune response. The antigen can elicit a greater immune response when combined with the adjuvant as described above.

### (1) Viral Antigens

The antigen can be a viral antigen, or fragment thereof, or variant thereof. The viral antigen can be from a virus from one of the following families: Adenoviridae, Arenaviridae, Bunyaviridae, Caliciviridae, Coronaviridae, Filoviridae, Hepadnaviridae, Herpesviridae, Orthomyxoviridae, Papovaviridae, Paramyxoviridae, Parvoviridae, Picornaviridae, Poxviridae, Reoviridae, Retroviridae, Rhabdoviridae, or Togaviridae. The viral antigen can be from papilloma viruses, for example, human papillomoa virus (HPV), human immunodeficiency virus (HIV), polio virus, hepatitis viruses, for example, hepatitis A virus (HAV), hepatitis B virus (HBV), hepatitis C virus (HCV), hepatitis D virus (HDV), and hepatitis E virus (HEV), smallpox virus (Variola major and minor), vaccinia virus, influenza virus, rhinoviruses, dengue fever virus, equine encephalitis viruses, rubella virus, yellow fever virus, Norwalk virus, hepatitis A virus, human T-cell leukemia virus (HTLV-I), hairy cell leukemia virus (HTLV-II), California encephalitis virus, Hanta virus (hemorrhagic fever), rabies virus, Ebola fever virus, Marburg virus, measles virus, mumps virus, respiratory syncytial virus (RSV), herpes simplex 1 (oral herpes), herpes simplex 2 (genital herpes), herpes zoster (varicella-zoster, a.k.a., chickenpox), cytomegalovirus (CMV), for example human CMV, Epstein-Barr virus (EBV), flavivirus, foot and mouth disease virus, chikungunya virus, lassa virus, arenavirus, cancer causing virus, porcine reproductive and respiratory syndrome (PPRS) virus, porcine circovirus type 2 (PCV2), foot and mouth disease virus (FMDV), avian influenza virus, avian paramyxovirus type 1, also called Newcastle disease virus (NDV), avian metapnemovirus, Marek's disease virus, Gumboro disease virus, also called infectious bursal disease virus (IBDV), Bovine virus Diarrhea (BVD), Classical Swine Fever (CSF), Parvovirus, Pseudorabies virus, Sagiyama virus, Swine herpes virus, Swine Poxvirus, Swine influenza virus, Vesicular stomatitis virus or virus of exanthema of swine. The viral antigen can also be from Canine Distemper Virus, Canine respiratory coronavirus, Canine Parainfluenza virus, Canine adenovirus, Canine Parvovirus and Canine Reovirus.

### (a) Hepatitis Antigen

IL-17 can be associated or combined with a hepatitis virus antigen (i.e., hepatitis antigen), or fragment thereof, or variant thereof. The hepatitis antigen can be an antigen or immunogen from hepatitis A virus (HAV), hepatitis B virus (HBV), hepatitis C virus (HCV), hepatitis D virus (HDV), and/or hepatitis E virus (HEV). In some embodiments, the hepatitis antigen can be a nucleic acid molecule(s), such as a plasmid(s), which encodes one or more of the antigens from HAV, HBV, HCV, HDV, and HEV. The hepatitis antigen can be full-length or immunogenic fragments of full-length proteins.

The hepatitis antigen can comprise consensus sequences and/or modification for improved expression. Genetic modifications including codon optimization, RNA optimization, and the addition of a high efficient immunoglobulin leader sequence to increase the immunogenicity of the constructs can be included in the modified consensus sequences. The consensus hepatitis antigen may comprise a signal peptide such as an immunoglobulin signal peptide such as an IgE or IgG signal peptide, and in some embodiments, may comprise an HA tag. The immunogens can be designed to elicit stronger and broader cellular immune responses than corresponding codong optimized immunogens.

The hepatitis antigen can be an antigen from HAV. The hepatitis antigen can be a HAV capsid protein, a HAV non-structural protein, a fragment thereof, a variant thereof, or a combination thereof.

The hepatitis antigen can be an antigen from HCV. The hepatitis antigen can be a HCV nucleocapsid protein (i.e., core protein), a HCV envelope protein (e.g., E1 and E2), a HCV non-structural protein (e.g., NS1, NS2, NS3, NS4a, NS4b, NS5a, and NS5b), a fragment thereof, a variant thereof, or a combination thereof.

The hepatitis antigen can be an antigen from HDV. The hepatitis antigen can be a HDV delta antigen, fragment thereof, or variant thereof.

The hepatitis antigen can be an antigen from HEV. The hepatitis antigen can be a HEV capsid protein, fragment thereof, or variant thereof.

The hepatitis antigen can be an antigen from HBV. The hepatitis antigen can be a HBV core protein, a HBV surface protein, a HBV DNA polymerase, a HBV protein encoded by gene X, fragment thereof, variant thereof, or combination thereof. The hepatitis antigen can be a HBV genotype A core protein, a HBV genotype B core protein, a HBV genotype C core protein, a HBV genotype D core protein, a HBV genotype E core protein, a HBV genotype F core protein, a HBV genotype G core protein, a HBV genotype H core protein, a HBV genotype A surface protein, a HBV genotype B surface protein, a HBV genotype C surface protein, a HBV genotype D surface protein, a HBV genotype E surface protein, a HBV genotype F surface protein, a HBV genotype G surface protein, a HBV genotype H surface protein, fragment thereof, variant thereof, or combination thereof. The hepatitis antigen can be a consensus HBV core protein, or a consensus HBV surface protein.

In some embodiments, the hepatitis antigen can be a HBV genotype A consensus core DNA sequence construct, an IgE leader sequence linked to a consensus sequence for HBV genotype A core protein, or a HBV genotype A consensus core protein sequence.

In other embodiments, the hepatitis antigen can be a HBV genotype B consensus core DNA sequence construct, an IgE leader sequence linked to a consensus sequence for HBV genotype B core protein, or a HBV genotype B consensus core protein sequence.

In still other embodiments, the hepatitis antigen can be a HBV genotype C consensus core DNA sequence construct, an IgE leader sequence linked to a consensus sequence for HBV genotype C core protein, or a HBV genotype C consensus core protein sequence.

In some embodiments, the hepatitis antigen can be a HBV genotype D consensus core DNA sequence construct, an IgE leader sequence linked to a consensus sequence for HBV genotype D core protein, or a HBV genotype D consensus core protein sequence.

In other embodiments, the hepatitis antigen can be a HBV genotype E consensus core DNA sequence construct, an IgE leader sequence linked to a consensus sequence for HBV genotype E core protein, or a HBV genotype E consensus core protein sequence.

In some embodiments, the hepatitis antigen can be a HBV genotype F consensus core DNA sequence construct, an IgE leader sequence linked to a consensus sequence for HBV genotype F core protein, or a HBV genotype F consensus core protein sequence.

In other embodiments, the hepatitis antigen can be a HBV genotype G consensus core DNA sequence construct, an IgE leader sequence linked to a consensus sequence for HBV genotype G core protein, or a HBV genotype G consensus core protein sequence.

In some embodiments, the hepatitis antigen can be a HBV genotype H consensus core DNA sequence construct, an IgE leader sequence linked to a consensus sequence for HBV genotype H core protein, or a HBV genotype H consensus core protein sequence.

In still other embodiments, the hepatitis antigen can be a HBV genotype A consensus surface DNA sequence construct, an IgE leader sequence linked to a consensus sequence for HBV genotype A surface protein, or a HBV genotype A consensus surface protein sequence.

In some embodiments, the hepatitis antigen can be a HBV genotype B consensus surface DNA sequence construct, an IgE leader sequence linked to a consensus sequence for HBV genotype B surface protein, or a HBV genotype B consensus surface protein sequence.

In other embodiments, the hepatitis antigen can be a HBV genotype C consensus surface DNA sequence construct, an IgE leader sequence linked to a consensus sequence for HBV genotype C surface protein, or a HBV genotype C consensus surface protein sequence.

In still other embodiments, the hepatitis antigen can be a HBV genotype D consensus surface DNA sequence construct, an IgE leader sequence linked to a consensus sequence for HBV genotype D surface protein, or a HBV genotype D consensus surface protein sequence.

In some embodiments, the hepatitis antigen can be a HBV genotype E consensus surface DNA sequence construct, an IgE leader sequence linked to a consensus sequence for HBV genotype E surface protein, or a HBV genotype E consensus surface protein sequence.

In other embodiments, the hepatitis antigen can be a HBV genotype F consensus surface DNA sequence construct, an IgE leader sequence linked to a consensus sequence for HBV genotype F surface protein, or a HBV genotype F consensus surface protein sequence.

In still other embodiments, the hepatitis antigen can be a HBV genotype G consensus surface DNA sequence construct, an IgE leader sequence linked to a consensus sequence for HBV genotype G surface protein, or a HBV genotype G consensus surface protein sequence.

In other embodiments, the hepatitis antigen can be a HBV genotype H consensus surface DNA sequence construct, an IgE leader sequence linked to a consensus sequence for HBV genotype H surface protein, or a HBV genotype H consensus surface protein sequence.

### (b) Human Papilloma Virus (HPV) Antigen

IL-17 can be associated or combined with a human papilloma virus (HPV) antigen, or fragment thereof, or variant thereof. The HPV antigen can be from HPV types 16, 18, 31, 33, 35, 45, 52, and 58 which cause cervical cancer, rectal cancer, and/or other cancers. The HPV antigen can be from HPV types 6 and 11, which cause genital warts, and are known to be causes of head and neck cancer.

The HPV antigens can be the HPV E6 or E7 domains from each HPV type. For example, for HPV type 16 (HPV16), the HPV16 antigen can include the HPV16 E6 antigen, the HPV16 E7 antigen, fragments, variants, or combinations thereof. Similarly, the HPV antigen can be HPV 6 E6 and/or E7, HPV 11 E6 and/or E7, HPV 18 E6 and/or E7, HPV 31 E6 and/or E7, HPV 33 E6 and/or E7, HPV 52 E6 and/or E7, or HPV 58 E6 and/or E7, fragments, variants, or combinations thereof.

### (c) RSV Antigen

IL-17 can also be associated or combined with an RSV antigen or fragment thereof, or variant thereof. The RSV antigen can be a human RSV fusion protein (also referred to herein as “RSV F”, “RSV F protein” and “F protein”), or fragment or variant thereof. The human RSV fusion protein can be conserved between RSV subtypes A and B. The RSV antigen can be a RSV F protein, or fragment or variant thereof, from the RSV Long strain (GenBank AAX23994.1). The RSV antigen can be a RSV F protein from the RSV A2 strain (GenBank AAB59858.1), or a fragment or variant thereof. The RSV antigen can be a monomer, a dimer or trimer of the RSV F protein, or a fragment or variant thereof. The RSV antigen can be an optimized amino acid RSV F amino acid sequence, or fragment or variant thereof.

The postfusion form of RSV F elicits high titer neutralizing antibodies in immunized animals and protects the animals from RSV challenge. The present invention utilizes this immunoresponse in the claimed vaccines. According to the invention, the RSV F protein can be in a prefusion form or a postfusion form.

The RSV antigen can also be human RSV attachment glycoprotein (also referred to herein as “RSV G”, “RSV G protein” and “G protein”), or fragment or variant thereof. The human RSV G protein differs between RSV subtypes A and B. The antigen can be RSV G protein, or fragment or variant thereof, from the RSV Long strain (GenBank AAX23993). The RSV antigen can be RSV G protein from: the RSV subtype B isolate H5601, the RSV subtype B isolate H1068, the RSV subtype B isolate H5598, the RSV subtype B isolate H1123, or a fragment or variant thereof. The RSV antigen can be an optimized amino acid RSV G amino acid sequence, or fragment or variant thereof.

In other embodiments, the RSV antigen can be human RSV non-structural protein 1 (“NS1 protein”), or fragment or variant thereof. For example, the RSV antigen can be RSV NS1 protein, or fragment or variant thereof, from the RSV Long strain (GenBank AAX23987.1). The RSV antigen human can also be RSV non-structural protein 2 (“NS2 protein”), or fragment or variant thereof. For example, the RSV antigen can be RSV NS2 protein, or fragment or variant thereof, from the RSV Long strain (GenBank AAX23988.1). The RSV antigen can further be human RSV nucleocapsid (“N”) protein, or fragment or variant thereof. For example, the RSV antigen can be RSV N protein, or fragment or variant thereof, from the RSV Long strain (GenBank AAX23989.1). The RSV antigen can be human RSV Phosphoprotein (“P”) protein, or fragment or variant thereof. For example, the RSV antigen can be RSV P protein, or fragment or variant thereof, from the RSV Long strain (GenBank AAX23990.1). The RSV antigen also can be human RSV Matrix protein (“M”) protein, or fragment or variant thereof. For example, the RSV antigen can be RSV M protein, or fragment or variant thereof, from the RSV Long strain (GenBank AAX23991.1).

In still other embodiments, the RSV antigen can be human RSV small hydrophobic (“SH”) protein, or fragment or variant thereof. For example, the RSV antigen can be RSV SH protein, or fragment or variant thereof, from the RSV Long strain (GenBank AAX23992.1). The RSV antigen can also be human RSV Matrix protein2-1 (“M2-1”) protein, or fragment or variant thereof. For example, the RSV antigen can be RSV M2-1 protein, or fragment or variant thereof, from the RSV Long strain (GenBank AAX23995.1). The RSV antigen can further be human RSV Matrix protein 2-2 (“M2-2”) protein, or fragment or variant thereof. For example, the RSV antigen can be RSV M2-2 protein, or fragment or variant thereof, from the RSV Long strain (GenBank AAX23997.1). The RSV antigen human can be RSV Polymerase L (“L”) protein, or fragment or variant thereof. For example, the RSV antigen can be RSV L protein, or fragment or variant thereof, from the RSV Long strain (GenBank AAX23996.1).

In further embodiments, the RSV antigen can have an optimized amino acid sequence of NS1, NS2, N, P, M, SH, M2-1, M2-2, or L protein. The RSV antigen can be a human RSV protein or recombinant antigen, such as any one of the proteins encoded by the human RSV genome.

In other embodiments, the RSV antigen can be, but is not limited to, the RSV F protein from the RSV Long strain, the RSV G protein from the RSV Long strain, the optimized amino acid RSV G amino acid sequence, the human RSV genome of the RSV Long strain, the optimized amino acid RSV F amino acid sequence, the RSV NS1 protein from the RSV Long strain, the RSV NS2 protein from the RSV Long strain, the RSV N protein from the RSV Long strain, the RSV P protein from the RSV Long strain, the RSV M protein from the RSV Long strain, the RSV SH protein from the RSV Long strain, the RSV M2-1 protein from the RSV Long strain, the RSV M2-2 protein from the RSV Long strain, the RSV L protein from the RSV Long strain, the RSV G protein from the RSV subtype B isolate H5601, the RSV G protein from the RSV subtype B isolate H1068, the RSV G protein from the RSV subtype B isolate H5598, the RSV G protein from the RSV subtype B isolate H1123, or fragment thereof, or variant thereof.

### (d) Influenza Antigen

IL-17 can be associated or combined with an influenza antigen or fragment thereof, or variant thereof. The influenza antigens are those capable of eliciting an immune response in a mammal against one or more influenza serotypes. The antigen can comprise the full length translation product HA0, subunit HA1, subunit HA2, a variant thereof, a fragment thereof or a combination thereof. The influenza hemagglutinin antigen can be a consensus sequence derived from multiple strains of influenza A serotype H1, a consensus sequence derived from multiple strains of influenza A serotype H2, a hybrid sequence containing portions of two different consensus sequences derived from different sets of multiple strains of influenza A serotype H1 or a consensus sequence derived from multiple strains of influenza B. The influenza hemagglutinin antigen can be from influenza B.

The influenza antigen can also contain at least one antigenic epitope that can be effective against particular influenza immunogens against which an immune response can be induced. The antigen may provide an entire repertoire of immunogenic sites and epitopes present in an intact influenza virus. The antigen may be a consensus hemagglutinin antigen sequence that can be derived from hemagglutinin antigen sequences from a plurality of influenza A virus strains of one serotype such as a plurality of influenza A virus strains of serotype H1 or of serotype H2. The antigen may be a hybrid consensus hemagglutinin antigen sequence that can be derived from combining two different consensus hemagglutinin antigen sequences or portions thereof. Each of two different consensus hemagglutinin antigen sequences may be derived from a different set of a plurality of influenza A virus strains of one serotype such as a plurality of influenza A virus strains of serotype H1. The antigen may be a consensus hemagglutinin antigen sequence that can be derived from hemagglutinin antigen sequences from a plurality of influenza B virus strains.

In some embodiments, the influenza antigen can be H1 HA, H2 HA, H3 HA, H5 HA, or a BHA antigen. Alternatively, the influenza antigen can be a consensus hemagglutinin antigen comprising a consensus H1 amino acid sequence or a consensus H2 amino acid sequence. The consensus hemagglutinin antigen may be a synthetic hybrid consensus H1 sequence comprising portions of two different consensus H1 sequences, which are each derived from a different set of sequences from the other. An example of a consensus HA antigen that is a synthetic hybrid consensus H1 protein is a protein comprising the U2 amino acid sequence. The consensus hemagglutinin antigen may be a consensus hemagglutinin protein derived from hemagglutinin sequences from influenza B strains, such as a protein comprising the consensus BHA amino acid sequence.

The consensus hemagglutinin antigen may further comprise one or more additional amino acid sequence elements. The consensus hemagglutinin antigen may further comprise on its N-terminal an IgE or IgG leader amino acid sequence.The consensus hemagglutinin antigen may further comprise an immunogenic tag which is a unique immunogenic epitope that can be detected by readily available antibodies. An example of such an immunogenic tag is the 9 amino acid influenza HA Tag which may be linked on the consensus hemagglutinin C terminus. In some embodiments, consensus hemagglutinin antigen may further comprise on its N-terminal an IgE or IgG leader amino acid sequence and on its C terminal an HA tag.

The consensus hemagglutinin antigen may be a consensus hemagglutinin protein that consists of consensus influenza amino acid sequences or fragments and variants thereof. The consensus hemagglutinin antigen may be a consensus hemagglutinin protein that comprises non-influenza protein sequences and influenza protein sequences or fragments and variants thereof.

Examples of a consensus H1 protein include those that may consist of the consensus H1 amino acid sequence or those that further comprise additional elements such as an IgE leader sequence, or an HA Tag or both an IgE leader sequence and an HA Tag.

Examples of consensus H2 proteins include those that may consist of the consensus H2 amino acid sequence or those that further comprise an IgE leader sequence, or an HA Tag, or both an IgE leader sequence and an HA Tag.

Examples of hybrid consensus H1 proteins include those that may consist of the consensus U2 amino acid sequence or those that further comprise an IgE leader sequence, or an HA Tag, or both an IgE leader sequence and an HA Tag.

Examples of hybrid consensus influenza B hemagglutinin proteins include those that may consist of the consensus BHA amino acid sequence or it may comprise an IgE leader sequence, or an HA Tag, or both an IgE leader sequence and an HA Tag.

The consensus hemagglutinin protein can be encoded by a consensus hemagglutinin nucleic acid, a variant thereof or a fragment thereof. Unlike the consensus hemagglutinin protein which may be a consensus sequence derived from a plurality of different hemagglutinin sequences from different strains and variants, the consensus hemagglutinin nucleic acid refers to a nucleic acid sequence that encodes a consensus protein sequence and the coding sequences used may differ from those used to encode the particular amino acid sequences in the plurality of different hemagglutinin sequences from which the consensus hemagglutinin protein sequence is derived. The consensus nucleic acid sequence may be codon optimized and/or RNA optimized. The consensus hemagglutinin nucleic acid sequence may comprise a Kozak's sequence in the 5′ untranslated region. The consensus hemagglutinin nucleic acid sequence may comprise nucleic acid sequences that encode a leader sequence. The coding sequence of an N terminal leader sequence is 5′ of the hemagglutinin coding sequence. The N-terminal leader can facilitate secretion. The N-terminal leader can be an IgE leader or an IgG leader. The consensus hemagglutinin nucleic acid sequence can comprise nucleic acid sequences that encode an immunogenic tag. The immunogenic tag can be on the C terminus of the protein and the sequence encoding it is 3′ of the HA coding sequence. The immunogenic tag provides a unique epitope for which there are readily available antibodies so that such antibodies can be used in assays to detect and confirm expression of the protein. The immunogenic tag can be an H Tag at the C-terminus of the protein.

### (e) Human Immunodeficiency Virus (HIV) Antigen

IL-17 can be associated or combined with an HIV antigen or fragment thereof, or variant thereof. HIV antigens can include modified consensus sequences for immunogens. Genetic modifications including codon optimization, RNA optimization, and the addition of a high efficient immunoglobin leader sequence to increase the immunogenicity of constructs can be included in the modified consensus sequences. The novel immunogens can be designed to elicit stronger and broader cellular immune responses than a corresponding codon optimized immunogens.

In some embodiments, the HIV antigen can be a subtype A consensus envelope DNA sequence construct, an IgE leader sequence linked to a consensus sequence for Subtype A envelope protein, or a subtype A consensus Envelope protein sequence.

In other embodiments, the HIV antigen can be a subtype B consensus envelope DNA sequence construct, an IgE leader sequence linked to a consensus sequence for Subtype B envelope protein, or an subtype B consensus Envelope protein sequence.

In still other embodiments, the HIV antigen can be a subtype C consensus envelope DNA sequence construct, an IgE leader sequence linked to a consensus sequence for subtype C envelope protein, or a subtype C consensus envelope protein sequence.

In further embodiments, the HIV antigen can be a subtype D consensus envelope DNA sequence construct, an IgE leader sequence linked to a consensus sequence for Subtype D envelope protein, or a subtype D consensus envelope protein sequence.

In some embodiments, the HIV antigen can be a subtype B Nef-Rev consensus envelope DNA sequence construct, an IgE leader sequence linked to a consensus sequence for Subtype B Nef-Rev protein, or a Subtype B Nef-Rev consensus protein sequence.

In other embodiments, the HIV antigen can be a Gag consensus DNA sequence of subtype A, B, C and D DNA sequence construct, an IgE leader sequence linked to a consensus sequence for Gag consensus subtype A, B, C and D protein, or a consensus Gag subtype A, B, C and D protein sequence.

In still other embodiments the HIV antigen can be a MPo1 DNA sequence or a MPo1 protein sequence. The HIV antigen can be nucleic acid or amino acid sequences of Env A, Env B, Env C, Env D, B Nef-Rev , Gag, or any combination thereof.

### (f) Porcine Reproductive and Respiratory Syndrome (PRRS) Virus

IL-17 can be associated or combined with a porcine reproductive and respiratory syndrome (PRRS) virus antigen, or fragment thereof, or variant thereof. The PRRS virus antigen can be from PRRS virus strain JXAI-R. In some embodiments, the PRRS antigen can be a glycoprotein selected from GP2 (ORF2a), GP3 (ORF3), GP4 (ORF4), or GP5 (ORFS) or a non-glycosylated protein selected from M (ORF6) and E (ORF2b), or combinations(s) thereof.

The PRRS virus antigen can comprise consensus sequences and/or modification for improved expression. Genetic modifications including codon optimization, RNA optimization, and the addition of a high efficient immunoglobulin leader sequence to increase the immunogenicity of the constructs can be included in the modified consensus sequences. The consensus PRRS virus antigen may comprise a signal peptide such as an immunoglobulin signal peptide such as an IgE or IgG signal peptide, and in some embodiments, may comprise an HA tag. The immunogens can be designed to elicit stronger and broader cellular immune responses than corresponding codon optimized immunogens.

### (g) Porcine Circovirus Type 2 (PCV2)

IL-17 can be associated or combined with a porcine circovirus type 2 (PCV2) antigen, or fragment thereof, or variant thereof. In some embodiments, the PCV2 antigen is selected from proteins ORF1, ORF2 and ORF3, alone or in combination(s). The PCV2 antigen can comprise consensus sequences and/or modification for improved expression. Genetic modifications including codon optimization, RNA optimization, and the addition of a high efficient immunoglobulin leader sequence to increase the immunogenicity of the constructs can be included in the modified consensus sequences. The consensus PCV2 antigen may comprise a signal peptide such as an immunoglobulin signal peptide such as an IgE or IgG signal peptide, and in some embodiments, may comprise an HA tag. The immunogens can be designed to elicit stronger and broader cellular immune responses than corresponding codon optimized immunogens.

### (h) Foot and Mouth Disease Virus (FMDV)

IL-17 can be associated or combined with a foot and mouth disease virus (FMDV) antigen, or fragment thereof, or variant thereof. In some embodiments, the antigen is VP1 or P1 proteins, alone or in combination(s). The FMDV antigen can comprise consensus sequences and/or modification for improved expression. Genetic modifications including codon optimization, RNA optimization, and the addition of a high efficient immunoglobulin leader sequence to increase the immunogenicity of the constructs can be included in the modified consensus sequences. The consensus FMDV antigen may comprise a signal peptide such as an immunoglobulin signal peptide such as an IgE or IgG signal peptide, and in some embodiments, may comprise an HA tag. The immunogens can be designed to elicit stronger and broader cellular immune responses than corresponding codon optimized immunogens.

### (2) Parasite Antigens

The antigen can be a parasite antigen or fragment or variant thereof. The parasite can be a protozoa, helminth, or ectoparasite. The helminth (i.e., worm) can be a flatworm (e.g., flukes and tapeworms), a thorny-headed worm, or a round worm (e.g., pinworms). The ectoparasite can be lice, fleas, ticks, and mites.

The parasite can be any parasite causing the following diseases: Acanthamoeba keratitis, Amoebiasis, Ascariasis, Babesiosis, Balantidiasis, Baylisascariasis, Chagas disease, Clonorchiasis, Cochliomyia, Cryptosporidiosis, Diphyllobothriasis, Dracunculiasis, Echinococcosis, Elephantiasis, Enterobiasis, Fascioliasis, Fasciolopsiasis, Filariasis, Giardiasis, Gnathostomiasis, Hymenolepiasis, Isosporiasis, Katayama fever, Leishmaniasis, Lyme disease, Malaria, Metagonimiasis, Myiasis, Onchocerciasis, Pediculosis, Scabies, Schistosomiasis, Sleeping sickness, Strongyloidiasis, Taeniasis, Toxocariasis, Toxoplasmosis, Trichinosis, and Trichuriasis.

The parasite can be Acanthamoeba, Anisakis, Ascaris lumbricoides, Botfly, Balantidium coli, Bedbug, Cestoda (tapeworm), Chiggers, Cochliomyia hominivorax, Entamoeba histolytica, Fasciola hepatica, Giardia lamblia, Hookworm, Leishmania, Linguatula serrata, Liver fluke, Loa loa, Paragonimus—lung fluke, Pinworm, Plasmodium falciparum, Schistosoma, Strongyloides stercoralis, Mite, Tapeworm, Toxoplasma gondii, Trypanosoma, Whipworm, or Wuchereria bancrofti.

### (a) Malaria Antigen

IL-17 can be associated or combined with a malaria antigen (i.e., PF antigen or PF immunogen), or fragment thereof, or variant thereof. The antigen can be from a parasite causing malaria. The malaria causing parasite can be Plasmodium falciparum. The Plasmodium falciparum antigen can include the circumsporozoite (CS) antigen.

In some embodiments, the malaria antigen can be nucleic acid molecules such as plasmids which encode one or more of the P. falciparum immunogens CS; LSA1; TRAP; CelTOS; and Ama1. The immunogens may be full length or immunogenic fragments of full length proteins. The immunogens comprise consensus sequences and/or modifications for improved expression.

In other embodiments, the malaria antigen can be a consensus sequence of TRAP, which is also referred to as SSP2, designed from a compilation of all full-length Plasmodium falciparum TRAP/SSP2 sequences in the GenBank database (28 sequences total). Consensus TRAP immunogens (i.e., ConTRAP immunogen) may comprise a signal peptide such as an immunoglobulin signal peptide such as an IgE or IgG signal peptide and in some embodiments, may comprise an HA Tag.

In still other embodiments, the malaria antigen can be CelTOS, which is also referred to as Ag2 and is a highly conserved Plasmodium antigen. Consensus CelTOS antigens (i.e., ConCelTOS immunogen) may comprise a signal peptide such as an immunoglobulin signal peptide such as an IgE or IgG signal peptide and in some embodiments, may comprise an HA Tag.

In further embodiments, the malaria antigen can be Ama1, which is a highly conserved Plasmodium antigen. The malaria antigen can also be a consensus sequence of Ama1 (i.e., ConAmaI immunogen) comprising in some instances, a signal peptide such as an immunoglobulin signal peptide such as an IgE or IgG signal peptide and in some embodiments, may comprise an HA Tag.

In some embodiments, the malaria antigen can be a consensus CS antigen (i.e., Consensus CS immunogen) comprising in some instances, a signal peptide such as an immunoglobulin signal peptide such as an IgE or IgG signal peptide and in some embodiments, may comprise an HA Tag.

In other embodiments, the malaria antigen can be a fusion protein comprising a combination of two or more of the PF proteins set forth herein. For example, fusion proteins may comprise two or more of Consensus CS immunogen, ConLSA1 immunogen, ConTRAP immunogen, ConCelTOS immunogen and ConAma1 immunogen linked directly adjacent to each other or linked with a spacer or one or more amino acids in between. In some embodiments, the fusion protein comprises two PF immunogens; in some embodiments the fusion protein comprises three PF immunogens, in some embodiments the fusion protein comprises four PF immunogens, and in some embodiments the fusion protein comprises five PF immunogens. Fusion proteins with two Consensus PF immunogens may comprise: CS and LSA1; CS and TRAP; CS and CelTOS; CS and Ama1; LSA1 and TRAP; LSA1 and CelTOS; LSA1 and Ama1; TRAP and CelTOS; TRAP and Ama1; or CelTOS and Ama1. Fusion proteins with three Consensus PF immunogens may comprise: CS, LSA1 and TRAP; CS, LSA1 and CelTOS; CS, LSA1 and Ama1; LSA1, TRAP and CelTOS; LSA1, TRAP and Ama1; or TRAP, CelTOS and Ama1. Fusion proteins with four Consensus PF immunogens may comprise: CS, LSA1, TRAP and CelTOS; CS, LSA1, TRAP and Amal; CS, LSA1, CelTOS and Ama1; CS, TRAP, CelTOS and Ama1; or LSA1, TRAP, CelTOS and Ama1. Fusion proteins with five Consensus PF immunogens may comprise CS or CS-alt, LSA1, TRAP, CelTOS and Ama1.

In some embodiments, the fusion proteins comprise a signal peptide linked to the N terminus. In some embodiments, the fusion proteins comprise multiple signal peptides linked to the N terminal of each Consensus PF immunogen. In some embodiments, a spacer may be included between PF immunogens of a fusion protein. In some embodiments, the spacer between PF immunogens of a fusion protein may be a proteolyic cleavage site. In some embodiments, the spacer may be a proteolyic cleavage site recognized by a protease found in cells to which the vaccine is intended to be administered and/or taken up. In some embodiments, a spacer may be included between PF immunogens of a fusion protein wherein the spacer is a proteolyic cleavage site recognized by a protease found in cells to which the vaccine is intended to be administered and/or taken up and the fusion proteins comprises multiple signal peptides linked to the N terminal of each Consensus PF immunogens such that upon cleavage the signal peptide of each Consensus PF immunogens translocates the Consensus PF immunogen to outside the cell.

### (3) Bacterial Antigens

The antigen can be a bacterial antigen or fragment or variant thereof. The bacterium can be from any one of the following phyla: Acidobacteria, Actinobacteria, Aquificae, Bacteroidetes, Caldiserica, Chlamydiae, Chlorobi, Chloroflexi, Chrysiogenetes, Cyanobacteria, Deferribacteres, Deinococcus-Thermus, Dictyoglomi, Elusimicrobia, Fibrobacteres, Firmicutes, Fusobacteria, Gemmatimonadetes, Lentisphaerae, Nitrospira, Planctomycetes, Proteobacteria, Spirochaetes, Synergistetes, Tenericutes, Thermodesulfobacteria, Thermotogae, and Verrucomicrobia.

The bacterium can be a gram positive bacterium or a gram negative bacterium. The bacterium can be an aerobic bacterium or an anerobic bacterium. The bacterium can be an autotrophic bacterium or a heterotrophic bacterium. The bacterium can be a mesophile, a neutrophile, an extremophile, an acidophile, an alkaliphile, a thermophile, a psychrophile, an halophile, or an osmophile.

The bacterium can be an anthrax bacterium, an antibiotic resistant bacterium, a disease causing bacterium, a food poisoning bacterium, an infectious bacterium, Salmonella bacterium, Staphylococcus bacterium, Streptococcus bacterium, or tetanus bacterium. The bacterium can be Actinobacillus pleuropneumoniae, Bacillus anthracis, Balantidium coli, Pasteurella multocida, Riemerella anatipestifer, Ornithobacterium rhinotracheale, Mycoplasma gallisepticum, Mycoplasma hyorhinis, Mycoplasma synoviae, Mycoplasma hyopneumoniae , Mycoplasma canis, Bordetella bronchiseptica, Brachyspira spp., preferably B. hyodyentheriae, B. pilosicoli, B. innocens, Brucella suis, preferably biovars 1, 2 and 3, Chlamydia and Chlamydophila sp., and preferably C. pecorum and C. abortus, Clostridium spp., preferably Cl. difficile, Cl. perfringens types A, B and C, Cl. novyi, Cl. septicum, Cl, tetani, Cryptosporidium parvum, Eimeria spp., Eperythrozoonis suis currently named Mycoplasma haemosuis, Erysipelothrix rhusiopathiae, Escherichia coli, Haemophilus parasuis, preferably subtypes 1 ,7 and 14, Isospora suis, Lawsonia intracellularis, Mannheimia haemolytica, Mycobacterium spp., preferably, M. avium, M. intracellular, M. tuberculosis and M. bovis, Pasteurella multocida, Salmonella spp., preferably, S. thyhimurium and S. choleraesuis, Staphylococcus spp., preferably, S. hyicus, methicillin-resistant Staphylococcus aureus (MRSA), Streptococcus spp., preferably Strep. Suis, Yersinia pestis.

### (a) Mycobacterium tuberculosis Antigens

IL-17 can be associated or combined with a Mycobacterium tuberculosis antigen (i.e., TB antigen or TB immunogen), or fragment thereof, or variant thereof. The TB antigen can be from the Ag85 family of TB antigens, for example, Ag85A and Ag85B. The TB antigen can be from the Esx family of TB antigens, for example, EsxA, EsxB, EsxC, EsxD, EsxE, EsxF, EsxH, EsxO, EsxQ, EsxR, EsxS, EsxT, EsxU, EsxV, or EsxW, or combination(s) thereof.

In some embodiments, the TB antigen can be nucleic acid molecules such as plasmids which encode one or more of the Mycobacterium tuberculosis immunogens from the Ag85 family and the Esx family. The immunogens can be full-length or immunogenic fragments of full-length proteins. The immunogens can comprise consensus sequences and/or modifications for improved expression. Consensus immunogens may comprise a signal peptide such as an immunoglobulin signal peptide such as an IgE or IgG signal peptide and in some embodiments, may comprise an HA tag.

### (b) Actinobacillus pleuropneumoniae Antigen

IL-17 can be associated or combined with a Actinobacillus pleuropneumoniae antigen, or fragment thereof, or variant thereof. In some embodiments, the antigen is selected from exotoxins ApxI, ApxII and ApxIII, alone or in combination(s). In some embodiments, the Actinobacillus pleuropneumoniae antigen can be nucleic acid molecules such as plasmids which encode one or more of Actinobacillus pleuropneumoniae antigens. The antigens can be full-length or immunogenic fragments of full-length proteins. The antigens can comprise consensus sequences and/or modifications for improved expression. Consensus antigens may comprise a signal peptide such as an immunoglobulin signal peptide such as an IgE or IgG signal peptide and in some embodiments, may comprise an HA tag.

### (4) Fungal Antigens

The antigen can be a fungal antigen or fragment or variant thereof. The fungus can be Aspergillus species, Blastomyces dermatitidis, Candida yeasts (e.g., Candida albicans), Coccidioides, Cryptococcus neoformans, Cryptococcus gattii, dermatophyte, Fusarium species, Histoplasma capsulatum, Mucoromycotina, Pneumocystis jirovecii, Sporothrix schenckii, Exserohilum, or Cladosporium.

### c. Vector

The vaccine can comprise one or more vectors that include a nucleic acid encoding the antigen and/or the adjuvant. The one or more vectors can be capable of expressing the antigen and/or the adjuvant. The vector can have a nucleic acid sequence containing an origin of replication. The vector can be a plasmid, bacteriophage, virus, bacterial artificial chromosome or yeast artificial chromosome. The vector can be either a self-replication extra chromosomal vector, or a vector which integrates into a host genome.

The one or more vectors can be an expression construct, which is generally a plasmid that is used to introduce a specific gene into a target cell. Once the expression vector is inside the cell, the protein that is encoded by the gene is produced by the cellular-transcription and translation machinery ribosomal complexes. The plasmid is frequently engineered to contain regulatory sequences that act as enhancer and promoter regions and lead to efficient transcription of the gene carried on the expression vector. The vectors of the present invention express large amounts of stable messenger RNA, and therefore proteins.

The vectors may have expression signals such as a strong promoter, a strong termination codon, adjustment of the distance between the promoter and the cloned gene, and the insertion of a transcription termination sequence and a PTIS (portable translation initiation sequence).

### (1) Expression Vectors

The vector can be a circular (e.g., plasmid) or a linear nucleic acid. The circular and linear nucleic acid are capable of directing expression of a particular nucleotide sequence in an appropriate subject cell. The vector can have a promoter operably linked to the antigen-encoding nucleotide sequence, or the adjuvant-encoding nucleotide sequence, which may be operably linked to termination signals. The vector can also contain sequences required for proper translation of the nucleotide sequence. The vector comprising the nucleotide sequence of interest may be chimeric, meaning that at least one of its components is heterologous with respect to at least one of its other components. The expression of the nucleotide sequence in the expression cassette may be under the control of a constitutive promoter or of an inducible promoter, which initiates transcription only when the host cell is exposed to some particular external stimulus. In the case of a multicellular organism, the promoter can also be specific to a particular tissue or organ or stage of development.

### (2) Circular and Linear Vectors

The vector may be circular plasmid, which may transform a target cell by integration into the cellular genome or exist extrachromosomally (e.g., autonomous replicating plasmid with an origin of replication).

The vector can be pVAX, pcDNA3.0, or provax, or any other expression vector capable of expressing DNA encoding the antigen, or the adjuvant and enabling a cell to translate the sequence to an antigen that is recognized by the immune system, or the adjuvant.

Also provided herein is a linear nucleic acid vaccine, or linear expression cassette (“LEC”), that is capable of being efficiently delivered to a subject via electroporation and expressing one or more desired antigens, or one or more desired adjuvants. The LEC may be any linear DNA devoid of any phosphate backbone. The DNA may encode one or more antigens, or one or more adjuvants. The LEC may contain a promoter, an intron, a stop codon, and/or a polyadenylation signal. The expression of the antigen, or the adjuvant may be controlled by the promoter. The LEC may not contain any antibiotic resistance genes and/or a phosphate backbone. The LEC may not contain other nucleic acid sequences unrelated to the desired antigen gene expression, or the desired adjuvant expression.

The LEC may be derived from any plasmid capable of being linearized. The plasmid may be capable of expressing the antigen, or the adjuvant. The plasmid may be capable of expressing the adjuvant IL-17. The plasmid can be pNP (Puerto Rico/34) or pM2 (New Caledonia/99). The plasmid may be WLV009, pVAX, pcDNA3.0, or provax, or any other expression vector capable of expressing DNA encoding the antigen, or encoding the adjuvant, and enabling a cell to translate the sequence to an antigen that is recognized by the immune system, or the adjuvant.

The LEC can be pcrM2. The LEC can be pcrNP. pcrNP and pcrMR can be derived from pNP (Puerto Rico/34) and pM2 (New Caledonia/99), respectively.

The expression vector may also be a recombinant viral vector. Such viral vector may be produced from non-pathogenic viruses such as HVT, MDV, adenoviruses, AAV, lentiviruses, swinepox viruses, etc.

### (3) Promoter, Intron, Stop Codon, and Polyadenylation Signal

The vector may have a promoter. A promoter may be any promoter that is capable of driving gene expression and regulating expression of the isolated nucleic acid. Such a promoter is a cis-acting sequence element required for transcription via a DNA dependent RNA polymerase, which transcribes the antigen sequence, or the adjuvant sequence described herein. Selection of the promoter used to direct expression of a heterologous nucleic acid depends on the particular application. The promoter may be positioned about the same distance from the transcription start in the vector as it is from the transcription start site in its natural setting. However, variation in this distance may be accommodated without loss of promoter function.

The promoter may be operably linked to the nucleic acid sequence encoding the antigen and signals required for efficient polyadenylation of the transcript, ribosome binding sites, and translation termination. The promoter may be operably linked to the nucleic acid sequence encoding the adjuvant and signals required for efficient polyadenylation of the transcript, ribosome binding sites, and translation termination.

The promoter may be a CMV promoter, SV40 early promoter, SV40 later promoter, metallothionein promoter, murine mammary tumor virus promoter, Rous sarcoma virus promoter, polyhedrin promoter, or another promoter shown effective for expression in eukaryotic cells.

The vector may include an enhancer and an intron with functional splice donor and acceptor sites. The vector may contain a transcription termination region downstream of the structural gene to provide for efficient termination. The termination region may be obtained from the same gene as the promoter sequence or may be obtained from different genes.

### d. Excipients and other Components of the Vaccine

The vaccine may further comprise a pharmaceutically acceptable excipient. The pharmaceutically acceptable excipient can be functional molecules such as vehicles, adjuvants other than IL-17, carriers, or diluents. The pharmaceutically acceptable excipient can be a transfection facilitating agent, which can include surface active agents, such as immune-stimulating complexes (ISCOMS), Freunds incomplete adjuvant, LPS analog including monophosphoryl lipid A, muramyl peptides, quinone analogs, vesicles such as squalene and squalene, hyaluronic acid, lipids, liposomes, calcium ions, viral proteins, polyanions, polycations, or nanoparticles, or other known transfection facilitating agents.

The transfection facilitating agent is a polyanion, polycation, including poly-L-glutamate (LGS), or lipid. The transfection facilitating agent is poly-L-glutamate, and the poly-L-glutamate may be present in the vaccine at a concentration less than 6 mg/ml. The transfection facilitating agent may also include surface active agents such as immune-stimulating complexes (ISCOMS), Freunds incomplete adjuvant, LPS analog including monophosphoryl lipid A, muramyl peptides, quinone analogs and vesicles such as squalene and squalene, and hyaluronic acid may also be used administered in conjunction with the genetic construct. The DNA plasmid vaccines may also include a transfection facilitating agent such as lipids, liposomes, including lecithin liposomes or other liposomes known in the art, as a DNA-liposome mixture (see for example W09324640), calcium ions, viral proteins, polyanions, polycations, or nanoparticles, or other known transfection facilitating agents. The transfection facilitating agent is a polyanion, polycation, including poly-L-glutamate (LGS), or lipid. Concentration of the transfection agent in the vaccine is less than 4 mg/ml, less than 2 mg/ml, less than 1 mg/ml, less than 0.750 mg/ml, less than 0.500 mg/ml, less than 0.250 mg/ml, less than 0.100 mg/ml, less than 0.050 mg/ml, or less than 0.010 mg/ml.

The pharmaceutically acceptable excipient can be an adjuvant in addition to IL-17. The additional adjuvant can be other genes that are expressed in an alternative plasmid or are delivered as proteins in combination with the plasmid above in the vaccine. The adjuvant may be selected from the group consisting of: α-interferon(IFN-α), (β-interferon (IFN-(β), γ-interferon, platelet derived growth factor (PDGF), TNFα, TNFβ, GM-CSF, epidermal growth factor (EGF), cutaneous T cell-attracting chemokine (CTACK), epithelial thymus-expressed chemokine (TECK), mucosae-associated epithelial chemokine (MEC), IL-12, IL-15, MHC, CD80, CD86 including IL-15 having the signal sequence deleted and optionally including the signal peptide from IgE. The adjuvant can be IL-12, IL-15, IL-28, CTACK, TECK, platelet derived growth factor (PDGF), TNFα, TNFβ, GM-CSF, epidermal growth factor (EGF), IL-1, IL-2, IL-4, IL-5, IL-6, IL-10, IL-12, IL-18, or a combination thereof.

Other genes that can be useful as adjuvants in addition to IL-17 include those encoding: MCP-1, MIP-1a, MIP-1p, IL-8, RANTES, L-selectin, P-selectin, E-selectin, CD34, GlyCAM-1, MadCAM-1, LFA-1, VLA-1, Mac-1, p150.95, PECAM, ICAM-1, ICAM-2, ICAM-3, CD2, LFA-3, M-CSF, G-CSF, IL-4, mutant forms of IL-18, CD40, CD4OL, vascular growth factor, fibroblast growth factor, IL-7, IL-22, nerve growth factor, vascular endothelial growth factor, Fas, TNF receptor, Flt, Apo-1, p55, WSL-1, DR3, TRAMP, Apo-3, AIR, LARD, NGRF, DR4, DR5, KILLER, TRAIL-R2, TRICK2, DR6, Caspase ICE, Fos, c-jun, Sp-1, Ap-1, Ap-2, p38, p65Rel, MyD88, IRAK, TRAF6, IkB, Inactive NIK, SAP K, SAP-1, JNK, interferon response genes, NFkB, Bax, TRAIL, TRAILrec, TRAILrecDRC5, TRAIL-R3, TRAIL-R4, RANK, RANK LIGAND, Ox40, Ox40 LIGAND, NKG2D, MICA, MICB, NKG2A, NKG2B, NKG2C, NKG2E, NKG2F, TAP1, TAP2 and functional fragments thereof.

The vaccine may further comprise a genetic vaccine facilitator agent as described in U.S. Ser. No. 021,579 filed Apr. 1, 1994, which is fully incorporated by reference.

In a particular embodiment, the vaccine of the invention comprises IL-17 as the sole adjuvant.

The vaccine can be formulated according to the mode of administration to be used. An injectable vaccine pharmaceutical composition can be sterile, pyrogen free and particulate free. An isotonic formulation or solution can be used. Additives for isotonicity can include sodium chloride, dextrose, mannitol, sorbitol, and lactose. The vaccine can comprise a vasoconstriction agent. The isotonic solutions can include phosphate buffered saline. Vaccine can further comprise stabilizers including gelatin and albumin. The stabilizers can allow the formulation to be stable at room or ambient temperature for extended periods of time, including LGS or polycations or polyanions.

A further object of the invention relates to a method for preparing a composition, comprising mixing an antigen and an IL-17 protein or nucleic acid, optionally in the presence of an acceptable excipient. In a particular embodiment, the method comprises mixing 1 μg to 10 mg of an antigen with 0.001 μg to about 100 μg of an IL-17 protein or nucleic acid.

### 3. Method of Vaccination

The present invention is also directed to a method of vaccinating or of increasing an immune response in a subject. Increasing the immune response can be used to treat and/or prevent disease in the subject. The method can include administering the herein disclosed vaccine to the subject. The subject administered the vaccine can have an increased or boosted immune response as compared to a subject administered the antigen alone. In some embodiments, the immune response can be increased by about 0.5-fold to about 15-fold, about 0.5-fold to about 10-fold, or about 0.5-fold to about 8-fold. Alternatively, the immune response in the subject administered the vaccine can be increased by at least about 0.5-fold, at least about 1.0-fold, at least about 1.5-fold, at least about 2.0-fold, at least about 2.5-fold, at least about 3.0-fold, at least about 3.5-fold, at least about 4.0-fold, at least about 4.5-fold, at least about 5.0-fold, at least about 5.5-fold, at least about 6.0-fold, at least about 6.5-fold, at least about 7.0-fold, at least about 7.5-fold, at least about 8.0-fold, at least about 8.5-fold, at least about 9.0-fold, at least about 9.5-fold, at least about 10.0-fold, at least about 10.5-fold, at least about 11.0-fold, at least about 11.5-fold, at least about 12.0-fold, at least about 12.5-fold, at least about 13.0-fold, at least about 13.5-fold, at least about 14.0-fold, at least about 14.5-fold, or at least about 15.0-fold.

In still other alternative embodiments, the immune response in the subject administered the vaccine can be increased about 50% to about 1500%, about 50% to about 1000%, or about 50% to about 800%. In other embodiments, the immune response in the subject administered the vaccine can be increased by at least about 50%, at least about 100%, at least about 150%, at least about 200%, at least about 250%, at least about 300%, at least about 350%, at least about 400%, at least about 450%, at least about 500%, at least about 550%, at least about 600%, at least about 650%, at least about 700%, at least about 750%, at least about 800%, at least about 850%, at least about 900%, at least about 950%, at least about 1000%, at least about 1050%, at least about 1100%, at least about 1150%, at least about 1200%, at least about 1250%, at least about 1300%, at least about 1350%, at least about 1450%, or at least about 1500%.

The invention also relates to a method for vaccinating a subject, or for inducing or increasing an immune response in a subject, the method comprising administering to the subject in need thereof an antigen and an IL-17 protein or nucleic acid. The antigen and the IL-17 protein or nucleic acid may be administered together or separately, simultaneously or sequentially. In a particular embodiment, the IL-17 protein or nucleic acid is administered before the antigen. Alternatively, or in addition, the IL-17 protein or nucleic acid and the antigen may be administered together. Furthermore, depending on the subject and conditions, the antigen and/or the IL-17 protein or nucleic acid may be administered repeatedly.

The vaccine dose can be between 1 μg to 10 mg active component/kg body weight/time, and can be 20 μg to 10 mg component/kg body weight/time. The vaccine can be administered every 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days. The number of vaccine doses for effective treatment can be 1, 2, 3, 4, 5, 6, 7, 8, 9, or 10.

### a. Administration

The vaccine can be formulated in accordance with standard techniques well known to those skilled in the pharmaceutical art. Such compositions can be administered in dosages and by techniques well known to those skilled in the medical arts taking into consideration such factors as the age, sex, weight, and condition of the particular subject, and the route of administration. The subject can be a mammal, such as a human, or a non-human, such as a horse, a cow, a pig, a sheep, a cat, a dog, a rat, an avian or a mouse.

The vaccine can be administered prophylactically or therapeutically. In prophylactic administration, the vaccines can be administered in an amount sufficient to induce an immune response. In therapeutic applications, the vaccines are administered to a subject in need thereof in an amount sufficient to elicit a therapeutic effect. An amount adequate to accomplish this is defined as “therapeutically effective dose.” Amounts effective for this use will depend on, e.g., the particular composition of the vaccine regimen administered, the manner of administration, the stage and severity of the disease, the general state of health of the patient, and the judgment of the prescribing physician.

The vaccine can be administered by methods well known in the art as described in Donnelly et al. (Ann. Rev. Immunol. 15:617-648 (1997)); Felgner et al. (U.S. Pat. No. 5,580,859, issued Dec. 3, 1996); Felgner (U.S. Pat. No. 5,703,055, issued Dec. 30, 1997); and Carson et al. (U.S. Pat. No. 5,679,647, issued Oct. 21, 1997), the contents of all of which are incorporated herein by reference in their entirety. The DNA of the vaccine can be complexed to particles or beads that can be administered to an individual, for example, using a vaccine gun. One skilled in the art would know that the choice of a pharmaceutically acceptable carrier, including a physiologically acceptable compound, depends, for example, on the route of administration of the expression vector.

The vaccine can be delivered via a variety of routes. Typical delivery routes include parenteral administration, e.g., intradermal, intramuscular or subcutaneous delivery. Other routes include oral administration, intranasal, and intravaginal routes. For the DNA of the vaccine in particular, the vaccine can be delivered to the interstitial spaces of tissues of an individual (Felgner et al., U.S. Pat. Nos. 5,580,859 and 5,703,055, the contents of all of which are incorporated herein by reference in their entirety). The vaccine can also be administered to muscle, or can be administered via intradermal or subcutaneous injections, or transdermally, such as by iontophoresis. Epidermal administration of the vaccine can also be employed. Epidermal administration can involve mechanically or chemically irritating the outermost layer of epidermis to stimulate an immune response to the irritant (Carson et al., U.S. Pat. No. 5,679,647, the contents of which are incorporated herein by reference in its entirety).

The vaccine can also be formulated for administration via the nasal passages. Formulations suitable for nasal administration, wherein the carrier is a solid, can include a coarse powder having a particle size, for example, in the range of about 10 to about 500 microns which is administered in the manner in which snuff is taken, i.e., by rapid inhalation through the nasal passage from a container of the powder held close up to the nose. The formulation can be a nasal spray, nasal drops, or by aerosol administration by nebulizer. The formulation can include aqueous or oily solutions of the vaccine.

The vaccine can be a liquid preparation such as a suspension, syrup or elixir. The vaccine can also be a preparation for parenteral, subcutaneous, intradermal, intramuscular or intravenous administration (e.g., injectable administration), such as a sterile suspension or emulsion.

The vaccine can be incorporated into liposomes, microspheres or other polymer matrices (Feigner et al., U.S. Pat. No. 5,703,055; Gregoriadis, Liposome Technology, Vols. Ito III (2nd ed. 1993), the contents of which are incorporated herein by reference in their entirety). Liposomes can consist of phospholipids or other lipids, and can be nontoxic, physiologically acceptable and metabolizable carriers that are relatively simple to make and administer.

The vaccine can be administered via electroporation, such as by a method described in U.S. Pat. No. 7,664,545, the contents of which are incorporated herein by reference. The electroporation can be by a method and/or apparatus described in U.S. Pat. Nos. 6,302,874; 5,676,646; 6,241,701; 6,233,482; 6,216,034; 6,208,893; 6,192,270; 6,181,964; 6,150,148; 6,120,493; 6,096,020; 6,068,650; and 5,702,359, the contents of which are incorporated herein by reference in their entirety. The electroporation may be carried out via a minimally invasive device.

The minimally invasive electroporation device (“MID”) may be an apparatus for injecting the vaccine described above and associated fluid into body tissue. The device may comprise a hollow needle, DNA cassette, and fluid delivery means, wherein the device is adapted to actuate the fluid delivery means in use so as to concurrently (for example, automatically) inject DNA into body tissue during insertion of the needle into the said body tissue. This has the advantage that the ability to inject the DNA and associated fluid gradually while the needle is being inserted leads to a more even distribution of the fluid through the body tissue. The pain experienced during injection may be reduced due to the distribution of the DNA being injected over a larger area.

The MID may inject the vaccine into tissue without the use of a needle. The MID may inject the vaccine as a small stream or jet with such force that the vaccine pierces the surface of the tissue and enters the underlying tissue and/or muscle. The force behind the small stream or jet may be provided by expansion of a compressed gas, such as carbon dioxide through a micro-orifice within a fraction of a second. Examples of minimally invasive electroporation devices, and methods of using them, are described in published U.S. Patent Application No. 20080234655; U.S. Pat. No. 6,520,950; U.S. Pat. No. 7,171,264; U.S. Pat. No. 6,208,893; U.S. Pat. No. 6,009,347; U.S. Pat. No. 6,120,493; U.S. Pat. No. 7,245,963; U.S. Pat. No. 7,328,064; and U.S. Pat. No. 6,763,264, the contents of each of which are herein incorporated by reference.

The MID may comprise an injector that creates a high-speed jet of liquid that painlessly pierces the tissue. Such needle-free injectors are commercially available. Examples of needle-free injectors that can be utilized herein include those described in U.S. Pat. Nos. 3,805,783; 4,447,223; 5,505,697; and 4,342,310, the contents of each of which are herein incorporated by reference.

A desired vaccine in a form suitable for direct or indirect electrotransport may be introduced (e.g., injected) using a needle-free injector into the tissue to be treated, usually by contacting the tissue surface with the injector so as to actuate delivery of a jet of the agent, with sufficient force to cause penetration of the vaccine into the tissue. For example, if the tissue to be treated is mucosa, skin or muscle, the agent is projected towards the mucosal or skin surface with sufficient force to cause the agent to penetrate through the stratum corneum and into dermal layers, or into underlying tissue and muscle, respectively.

Needle-free injectors are well suited to deliver vaccines to all types of tissues, particularly to skin and mucosa. In some embodiments, a needle-free injector may be used to propel a liquid that contains the vaccine to the surface and into the subject's skin or mucosa. Representative examples of the various types of tissues that can be treated using the invention methods include pancreas, larynx, nasopharynx, hypopharynx, oropharynx, lip, throat, lung, heart, kidney, muscle, breast, colon, prostate, thymus, testis, skin, mucosal tissue, ovary, blood vessels, or any combination thereof.

The MID may have needle electrodes that electroporate the tissue. By pulsing between multiple pairs of electrodes in a multiple electrode array, for example set up in rectangular or square patterns, provides improved results over that of pulsing between a pair of electrodes. Disclosed, for example, in U.S. Pat. No. 5,702,359 entitled “Needle Electrodes for Mediated Delivery of Drugs and Genes” is an array of needles wherein a plurality of pairs of needles may be pulsed during the therapeutic treatment. In that application, which is incorporated herein by reference as though fully set forth, needles were disposed in a circular array, but have connectors and switching apparatus enabling a pulsing between opposing pairs of needle electrodes. A pair of needle electrodes for delivering recombinant expression vectors to cells may be used. Such a device and system is described in U.S. Pat. No. 6,763,264, the contents of which are herein incorporated by reference. Alternatively, a single needle device may be used that allows injection of the DNA and electroporation with a single needle resembling a normal injection needle and applies pulses of lower voltage than those delivered by presently used devices, thus reducing the electrical sensation experienced by the patient.

The MID may comprise one or more electrode arrays. The arrays may comprise two or more needles of the same diameter or different diameters. The needles may be evenly or unevenly spaced apart. The needles may be between 0.005 inches and 0.03 inches, between 0.01 inches and 0.025 inches; or between 0.015 inches and 0.020 inches. The needle may be 0.0175 inches in diameter. The needles may be 0.5 mm, 1.0 mm, 1.5 mm, 2.0 mm, 2.5 mm, 3.0 mm, 3.5 mm, 4.0 mm, or more spaced apart.

The MID may consist of a pulse generator and a two or more-needle vaccine injectors that deliver the vaccine and electroporation pulses in a single step. The pulse generator may allow for flexible programming of pulse and injection parameters via a flash card operated personal computer, as well as comprehensive recording and storage of electroporation and patient data. The pulse generator may deliver a variety of volt pulses during short periods of time. For example, the pulse generator may deliver three 15 volt pulses of 100 ms in duration. An example of such a MID is the Elgen 1000 system by Inovio Biomedical Corporation, which is described in U.S. Pat. No. 7,328,064, the contents of which are herein incorporated by reference.

The MID may be a CELLECTRA (Inovio Pharmaceuticals, Blue Bell Pa.) device and system, which is a modular electrode system, that facilitates the introduction of a macromolecule, such as a DNA, into cells of a selected tissue in a body or plant. The modular electrode system may comprise a plurality of needle electrodes; a hypodermic needle; an electrical connector that provides a conductive link from a programmable constant-current pulse controller to the plurality of needle electrodes; and a power source. An operator can grasp the plurality of needle electrodes that are mounted on a support structure and firmly insert them into the selected tissue in a body or plant. The macromolecules are then delivered via the hypodermic needle into the selected tissue. The programmable constant-current pulse controller is activated and constant-current electrical pulse is applied to the plurality of needle electrodes. The applied constant-current electrical pulse facilitates the introduction of the macromolecule into the cell between the plurality of electrodes. Cell death due to overheating of cells is minimized by limiting the power dissipation in the tissue by virtue of constant-current pulses. The Cellectra device and system is described in U.S. Pat. No. 7,245,963, the contents of which are herein incorporated by reference.

The MID may be an Elgen 1000 system (Inovio Pharmaceuticals). The Elgen 1000 system may comprise device that provides a hollow needle; and fluid delivery means, wherein the apparatus is adapted to actuate the fluid delivery means in use so as to concurrently (for example automatically) inject fluid, the described vaccine herein, into body tissue during insertion of the needle into the said body tissue. The advantage is the ability to inject the fluid gradually while the needle is being inserted leads to a more even distribution of the fluid through the body tissue. It is also believed that the pain experienced during injection is reduced due to the distribution of the volume of fluid being injected over a larger area.

In addition, the automatic injection of fluid facilitates automatic monitoring and registration of an actual dose of fluid injected. This data can be stored by a control unit for documentation purposes if desired.

It will be appreciated that the rate of injection could be either linear or non-linear and that the injection may be carried out after the needles have been inserted through the skin of the subject to be treated and while they are inserted further into the body tissue.

Suitable tissues into which fluid may be injected by the apparatus of the present invention include tumor tissue, skin or liver tissue but may be muscle tissue.

The apparatus further comprises needle insertion means for guiding insertion of the needle into the body tissue. The rate of fluid injection is controlled by the rate of needle insertion. This has the advantage that both the needle insertion and injection of fluid can be controlled such that the rate of insertion can be matched to the rate of injection as desired. It also makes the apparatus easier for a user to operate. If desired means for automatically inserting the needle into body tissue could be provided.

A user could choose when to commence injection of fluid. Ideally however, injection is commenced when the tip of the needle has reached muscle tissue and the apparatus may include means for sensing when the needle has been inserted to a sufficient depth for injection of the fluid to commence. This means that injection of fluid can be prompted to commence automatically when the needle has reached a desired depth (which will normally be the depth at which muscle tissue begins). The depth at which muscle tissue begins could for example be taken to be a preset needle insertion depth such as a value of 4 mm which would be deemed sufficient for the needle to get through the skin layer.

The sensing means may comprise an ultrasound probe. The sensing means may comprise a means for sensing a change in impedance or resistance. In this case, the means may not as such record the depth of the needle in the body tissue but will rather be adapted to sense a change in impedance or resistance as the needle moves from a different type of body tissue into muscle. Either of these alternatives provides a relatively accurate and simple to operate means of sensing that injection may commence. The depth of insertion of the needle can further be recorded if desired and could be used to control injection of fluid such that the volume of fluid to be injected is determined as the depth of needle insertion is being recorded.

The apparatus may further comprise: a base for supporting the needle; and a housing for receiving the base therein, wherein the base is moveable relative to the housing such that the needle is retracted within the housing when the base is in a first rearward position relative to the housing and the needle extends out of the housing when the base is in a second forward position within the housing. This is advantageous for a user as the housing can be lined up on the skin of a patient, and the needles can then be inserted into the patient's skin by moving the housing relative to the base.

As stated above, it is desirable to achieve a controlled rate of fluid injection such that the fluid is evenly distributed over the length of the needle as it is inserted into the skin. The fluid delivery means may comprise piston driving means adapted to inject fluid at a controlled rate. The piston driving means could for example be activated by a servo motor. However, the piston driving means may be actuated by the base being moved in the axial direction relative to the housing. It will be appreciated that alternative means for fluid delivery could be provided. Thus, for example, a closed container which can be squeezed for fluid delivery at a controlled or non-controlled rate could be provided in the place of a syringe and piston system.

The apparatus described above could be used for any type of injection. It is however envisaged to be particularly useful in the field of electroporation and so it may further comprises means for applying a voltage to the needle. This allows the needle to be used not only for injection but also as an electrode during, electroporation. This is particularly advantageous as it means that the electric field is applied to the same area as the injected fluid. There has traditionally been a problem with electroporation in that it is very difficult to accurately align an electrode with previously injected fluid and so user's have tended to inject a larger volume of fluid than is required over a larger area and to apply an electric field over a higher area to attempt to guarantee an overlap between the injected substance and the electric field. Using the present invention, both the volume of fluid injected and the size of electric field applied may be reduced while achieving a good fit between the electric field and the fluid.

A further object of the invention is a kit comprising a first and a second container, said first container comprising an antigen and said second container comprising an IL-17 protein or nucleic acid.

The invention also relates to a kit comprising a first container comprising an antigen in admixture with an IL-17 protein or nucleic acid, and a means for administering said admixture to a subject. The means may be any injectible device, a syringe, patch, a spray, and the like.

The present invention has multiple aspects, illustrated by the following non-limiting examples.

### EXAMPLES

### Example 1

### IL-6 and TNF-α

IL-17A may induce cytokines that are part of the innate immune response, for example, interleukin-6 (IL-6) and tumor necrosis factor alpha (TNF-α). To determine if IL-17A can enhance or increase the immune response to a viral antigen (e.g., a “live” or infectious influenza viral antigen), and thus act as an adjuvant, IL-17A protein was administered prior to administration of the infectious viral antigen. Accordingly, the vaccine included rIL-17A and influenza A virus H5N1, in which the two components of the vaccine were administered at different times. Specifically, levels of IL-6 and TNF-α were measured in sera collected from mice receiving vaccine including IL-17A and vaccine not including IL-17A.

Two groups of C57BL/6 mice were administered (or pre-treated with) intraperitoneally (i.p.) 0.1 μg and 0.5 μg, respectively, of recombinant IL-17A (rIL-17A) on day 7 and day 2 before administration of (or challenged with) the infectious viral antigen. In particular, rIL-17A was a mouse IL-17A homodimer and the infectious viral antigen was influenza A virus H5N1. The two groups of mice were administered 5 LD₅₀ of influenza A virus H5N1 on day 0. The H5N1 virus was administered nasally to the mice.

A third group of C57BL/6 mice served as a control and was not administered rIL-17A nor influenza A virus H5N1 (i.e., naïve mice). At 6 dpi, sera were collected from the mice and IL-6 and TNF-α levels were measured in the collected sera.

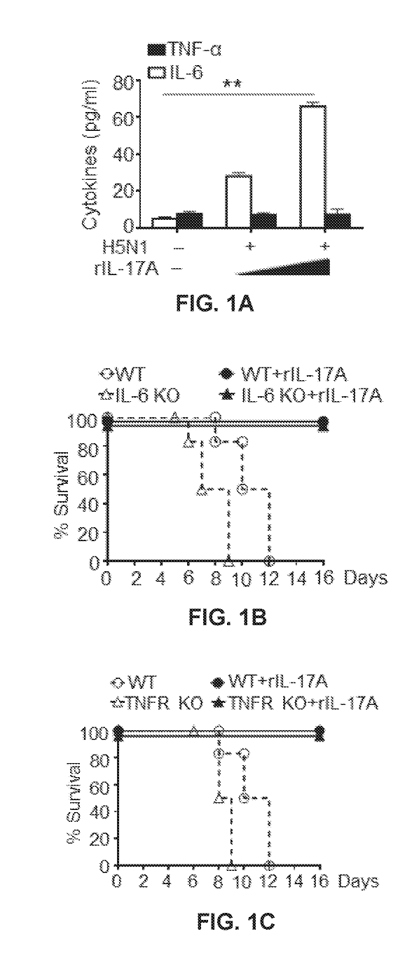

Compared to naïve mice, mice pre-treated with rIL-17A before viral challenge had increased levels of IL-6 in the sera (FIG. 1A, **p<0.01). The increase in IL-6 levels was dependent upon the dose of rIL-17A given in the pre-treatment, in which a higher dose of rIL-17A resulted in higher serum levels of IL-6. TNF-α levels, however, were unaltered by pre-treatment with rIL-17A and challenge with influenza A virus H5N1.

Additionally, wild-type (i.e.,C57BL/6), IL-6 knockout, and TNFR1/2 (i.e., TNF-α) knockout mice were challenged with influenza A virus H5N1 alone or in combination with rIL-17A pre-treatment. Mortality after infection was then measured. As shown in FIG. 1B, wild-type mice had died by about 9 dpi while IL-6 knockout mice died by 12 dpi. 100% of the wild-type and IL-6 knockout mice pre-treated with rIL-17A, however, survived the challenge with influenza A virus H5N1. These data indicated that while IL-6 levels increased in a dose dependent manner after pre-treatment with rIL-17A, IL-6 was not required for the increased immune response supported by the vaccine including IL-17A.

As shown in FIG. 1C, wild-type and TNFR1/1 knockout mice had died by 9 dpi and 12 dpi, respectively. 100% of the wild-type and TNFR1/2 knockout mice pre-treated with rIL-17A survived the challenge with influenza A virus H5N1. These data indicated that TNF-α levels do not change in response to pre-treatment with rIL-17A nor is TNF-α required for increased immune response supported by the vaccine including IL-17A.

In summary, the vaccine including IL-17A increased the immune response to the H5N1 virus (as compared to the vaccine not including IL-17A) independent of IL-6 and TNF-α.

### Example 2

### IFN-γ and IL-4

Cytokines, for example, interleukin-4 (IL-4) and interferon-gamma (IFN-γ), may control infection by influenza A virus. To examine if IL-17A as an adjuvant increased the immune response to the antigen via IL-4 and IFN-γ, the levels of IL-4 and IFN-γ were measured in sera collected from mice administered the vaccine including IL-17A and influenza A virus H5N1 or H7N9 and the vaccine not including IL-17A (i.e., influenza A virus H5N1 or H7N9 only).

Specifically, two groups of C57BL/6 mice were pre-treated i.p. with 0.1 μg and 0.5 μg, respectively, of rIL-17A on day 7 and day 2 before viral infection. A third group of C57BL/6 mice did not receive rIL-17A pre-treatment. The three groups of mice were challenged with 5 LD₅₀ of influenza A virus H5N1 on day 0. The H5N1 virus was administered nasally to the mice. A fourth group of C57BL/6 mice served as a control and was not pre-treated with rIL-17A nor challenged with influenza A virus H5N1 (i.e., naive mice). At 6 dpi, sera were collected from the mice and IL-4 and IFNγ levels were measured in the collected sera.

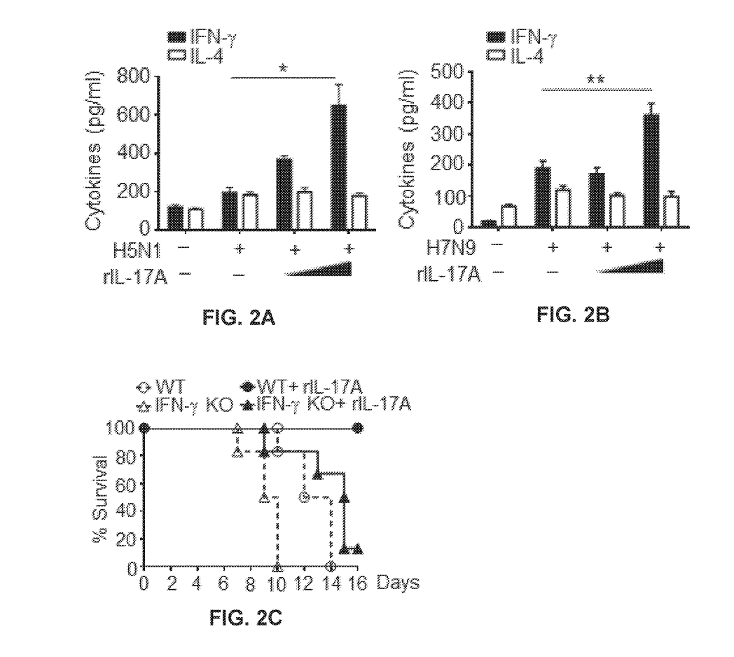

As shown in FIG. 2A, no significant difference in IL-4 levels was observed between the four groups of mice. The data in FIG. 2A are representative of three independent experiments. IFN-γ levels were increased in mice challenged with influenza A virus H5N1 as compared to naive mice. IFN-γ levels, however, significantly increased in a dose dependent manner in response to rIL-17A pre-treatment as compared to mice challenged with influenza A virus H5N1 (FIG. 2A, *p<0.05 (unpaired student's t-test)). Mice pre-treated with 0.5 μg of rIL-17A had higher serum levels of IFN-γ than mice pre-treated with 0.1 μg of rIL-17A, mice challenged with H5N1, and naïve mice (FIG. 2A, about 1.6-fold, about 3.25-fold, and about 6.5-fold, respectively, higher IFN-γ levels). These data indicated that IFN-γ, but not IL-4, levels in serum were significantly increased in response to rIL-17A pre-treatment in a dose dependent manner.

To further examine IL-4 and IFN-γ levels, the above experiment was repeated, but with influenza A virus H7N9 instead of influenza A virus H5N1. Specifically, two groups of C57BL/6 mice were pre-treated i.p. with 0.1 μg and 0.5 μg, respectively, of rIL-17A on day 7 and day 2 before viral infection. A third group of C57BL/6 mice did not receive rIL-17A pre-treatment. The three groups of mice were challenged with 10 LD₅₀ of influenza A virus H7N9 on day 0. The H7N9 virus was administered nasally to the mice. A fourth group of C57BL/6 mice served as a control and was not pre-treated with rIL-17A nor challenged with influenza A virus H7N9 (i.e., naïve mice). At 6 dpi, sera were collected from the mice and IL-4 and IFN-γ levels were measured in the collected sera.

As shown in FIG. 2B, no difference in IL-4 levels was observed between the four groups of mice. The data in FIG. 2B are representative of three independent experiments. IFN-γ levels were comparable between mice challenged with influenza A virus H7N9 and mice pre-treated with 0.1 μg rIL-17 before viral challenge. IFN-γ levels, however, were significantly increased in mice pre-treated with 0.5 μg rIL-17A as compared to mice not receiving pre-treatment and mice pre-treated with 0.1 μg rIL-17A (FIG. 2B, **p<0.01 (unpaired student's t-test)). In particular, mice pre-treated with 0.5 μg rIL-17A had about 1.9-fold higher levels of IFN-γ as compared to mice not receiving pre-treatment with rIL-17A. These data indicated that IFN-γ, but not IL-4, levels in serum significantly increased in response to rIL-17A pre-treatment in a dose dependent manner.

To further examine IFN-γ, wild-type (i.e., C56BL/6) and IFN-γ knockout mice were challenged with influenza A virus H5N1 alone or in combination with rIL-17A pre-treatment. Specifically, one group of wild-type mice (n=6) and one group of IFN-γ knockout mice (n=6) were pre-treated i.p. with 0.5 μg of rIL-17A on day 7 and day 2 before viral infection. A second group of wild-type mice (n=6) and a second group of IFN-γ knockout mice (n=6) did not receive rIL-17A pre-treatment. The four groups of mice were challenged with 5 LD₅₀ of influenza A virus H5N1 on day 0. The H5N1 virus was administered nasally to the mice. Mice were monitored daily for mortality 16 days after infection.

As shown in FIG. 2C, wild-type and IFN-γ knockout mice challenged with influenza A virus H5N1 died by 14 dpi and 10 dpi, respectively. This data indicated that the IFN-γ knockout mice were more susceptible to H5N1 viral infection than wild-type mice. Additionally, pre-treatment of the IFN-γ knockout mice with rIL-17A delayed death as compared to the IFN-γ knockout mice that did not receive rIL-17A pre-treatment. By 16 dpi, about 15% of the IFN-γ knockout mice pre-treated with rIL-17A had survived challenge with influenza A virus H5N1. In contrast, 100% of wild-type mice pre-treated with rIL-17A survived the challenge with influenza A virus H5N1. These data indicated that the vaccine including IL-17A increased the immune response through IFN-γ. In other words, IFN-γ was needed for the increased immune response provided when the vaccine included IL-17A as an adjuvant.

In summary, IL-4 levels were unchanged in response to rIL-17A pre-treatment. IFN-γ levels, however, increased in response to rIL-17A pre-treatment in a dose dependent manner and the vaccine including IL-17A as an adjuvant supported an increased immune response via IFN-γ.

### Example 3

### IFN-γ produced by CD8⁺T cells and Natural Killer Cells

During viral infection, IFN-γ may be secreted by CD4⁺ T cells, CD8⁺ T cells, and/or natural killer (NK) cells. To determine the cell population(s) responsible for the increased levels of IFN-γ induced by the vaccine including rIL-17A as an adjuvant, IFN-γ positive cells were detected at 6 dpi.

Specifically, wild-type mice (i.e., C57BL/6) mice were pre-treated i.p. with 0.5 μg of rIL-17A on day 7 and day 2 before administration of the virus. A second group of C57BL/6 mice did not receive rIL-17A pre-treatment. These two groups of wild-type mice plus a group of IL-17A knockout mice were challenged with 5 LD₅₀ of influenza A virus H5N1 on day 0. The H5N1 virus was administered nasally to the mice. Another group of C57BL/6 mice served as a control and was not pre-treated with rIL-17A nor challenged with influenza A virus H5N1 (i.e., naïve mice). Splenocytes were isolated from the four groups of mice at 6 dpi and stimulated with formalin-inactivated influenza A virus H5N1 for 12 hours (h) in vitro. Secretion of IFN-γ by CD4⁺ T cells, CD8⁺ T cells, or NK cells was measured by flow cytometry, which detected intracellular staining of IFN-γ.

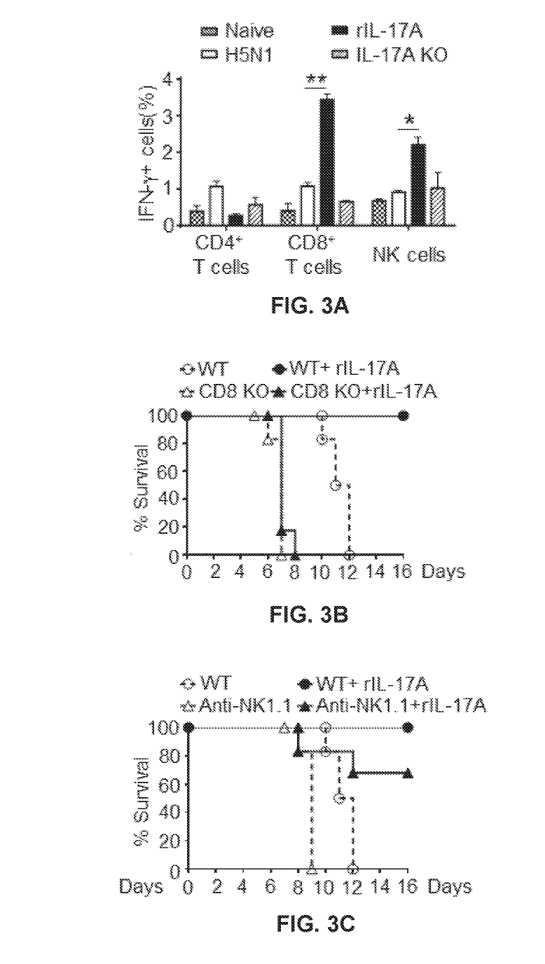

As shown in FIG. 3A, secretion of IFN-γ from CD4⁺ T cells isolated from C57BL/6 mice receiving the vaccine including rIL-17A and virus (i.e., pre-treated with rIL-17A (and challenged with virus)) was decreased as compared to IFN-γ secretion from CD4⁺ T cells isolated from mice receiving the vaccine that did not include rIL-17A (i.e., not receiving rIL-17A pre-treatment (but challenged with virus)). Accordingly, secretion of IFN-γ from CD4⁺ T cells decreased in response to rIL-17A pre-treatment. The data in FIG. 3A are representative of three independent experiments. In contrast, administration of the vaccine including rIL-17A (i.e., rIL-17A pre-treatment) significantly increased IFN-γ secretion from CD8⁺ T cells as compared to in the absence of rIL-17A in the vaccine (i.e., absence of rIL-17A pre-treatment) (FIG. 3A, **p<0.01). Administration of the vaccine including rIL-17A (i.e., rIL-17A pre-treatment) also significantly increased IFN-γ secretion from NK cells as compared to in the absence of rIL-17A in the vaccine (i.e., absence of rIL-17A pre-treatment) (FIG. 3A, *p<0.05). Accordingly, these data indicated that CD8⁺ T cells and NK cells significantly contributed to the increased levels of IFN-γ observed in the increased immune response supported by the vaccine including rIL-17A.

Because CD8⁺ T cells secreted IFN-y in response to rIL-17A pre-treatment, the ability of mice lacking CD8⁺ T cells (i.e., CD8 knockout mice) to withstand challenge with influenza A virus H5N1 was examined to determine if the vaccine including rIL-17A increased the immune response to viral antigen via CD8⁺ T cells. Specifically, wild-type (i.e., C57BL/6) mice and CD8 knockout mice were pre-treated i.p. with 0.5 μg of rIL-17A on day 7 and day 2 before administration of the virus. A second group of C57BL/6 mice and a second group of CD8 knockout mice did not receive rIL-17A pre-treatment. The four groups of mice were challenged with 5 LD₅₀ of influenza A virus H5N1 on day 0. The H5N1 virus was administered nasally to the mice. Mice were monitored daily for 16 days after infection for mortality.

As shown in FIG. 3B, wild-type and CD8 knockout mice challenged with influenza A virus H5N1 died at 12 dpi and 7 dpi, respectively. This data indicated that CD8 knockout mice were more susceptible to H5N1 viral infection than wild-type mice. Additionally, CD8 knockout mice pre-treated with rIL-17A died by 8 dpi, and thus, pre-treatment of CD8 knockout mice with rIL-17A delayed death by 1 day as compared to CD8 knockout mice that did not receive rIL-17A pre-treatment (FIG. 3B). As observed above, 100% of wild-type mice pre-treated with rIL-17A survived the challenge with influenza A virus H5N1. These data indicated that the vaccine including rIL-17A increased the immune response to the influenza A virus through CD8⁺ T cells. In other words, CD8⁺ T cells were needed for the increased immune response supported by the vaccine including IL-17A as an adjuvant.

As demonstrated above, IFN-γ was also secreted by NK cells in response to rIL-17A pre-treatment. Accordingly, the ability of mice depleted of NK cells (i.e., anti-NK 1.1 mice) to withstand challenge with influenza A virus H5N1 was examined to determine if the vaccine including rIL-17A as an adjuvant increased the immune response to the virus via NK cells. Specifically, four groups of C57BL/6 mice were examined. The first group of C57BL/6 mice were not depleted of NK cells nor pre-treated with rIL-17A. The second group of C57BL/6 mice was pre-treated i.p. with 0.5 μg rIL-17A on day 7 and day 2 before administration of the virus. The third group of C57BL/6 mice was injected i.p. with anti-NK 1.1 neutralizing monoclonal antibody on day 7, day 3, and day 1 before administration of the virus. The fourth group of C57BL/6 mice was injected i.p. with anti-NK 1.1 neutralizing monoclonal antibody on day 7, day 3, and day 1 before administration of the virus, and pre-treated i.p. with 0.5 μg of rIL-17A on day 7 and day 2 before administration of the virus. The four groups of mice were challenged with 5 LD₅₀ of influenza A virus H5N1 on day 0. The H5N1 virus was administered nasally to the mice. Mice were monitored daily for 16 days after infection for mortality.

As shown in FIG. 3C, wild-type and NK depleted (i.e., anti-NK 1.1) mice challenged with influenza A virus H5N1 died by 12 dpi and 9 dpi, respectively. This data indicated that NK depleted mice were more susceptible to H5N1 viral infection than wild-type mice. By 16 dpi, about 70% of NK depleted mice pre-treated with rIL-17A (i.e., anti-NK 1.1+rIL-17A) had survived the challenge with influenza A virus H5N1 (FIG. 3C). 100% of wild-type mice pre-treated with rIL-17A survived challenge with influenza A virus H5N1. These data indicated that NK cells played a smaller role (than CD8⁺ T cells) in facilitating the increased immune response provided by the vaccine including rIL-17A as an adjuvant because mice depleted of NK cells, but pre-treated with rIL-17A had a modestly reduced survival rate as compared to wild-type mice pre-treated with rIL-17A (i.e., about 70% vs. 100% survival rate to viral challenge).

In summary, IFN-γ secretion by CD8⁺ T cells and NK cells, but not CD4⁺ T cells, was significantly increased in response to rIL-17A pre-treatment. Accordingly, the vaccine including rIL-17A as an adjuvant increased the immune response to the virus via IFN-γ secretion by CD8⁺ T cells and NK cells, in which the CD8+ T cells contributed more to the increased immune response than the NK cells. The increased immune response supported by the vaccine including rIL-17A as an adjuvant was significantly reduced in the absence of CD8⁺ T cells, but modestly reduced in the absence of NK cells. Rather, in the absence of CD8⁺ T cells, rIL-17A pre-treatment delayed, but did not prevent, death. Together, these data indicated that IFN-γ producing CD8⁺ T cells significantly contributed to the increased immune response to the virus that was supported by the vaccine including rIL-17A as an adjuvant.

### Example 4

### Adoptive Transfer of CD8⁺ T cells

As demonstrated above, the vaccine including IL-17A as an adjuvant increased the immune response to live or infectious virus and such an increased immune response was achieved when the IL-17A adjuvant was administered before the virus. This increased immune response was facilitated by IFN-y producing CD8⁺ T cells.

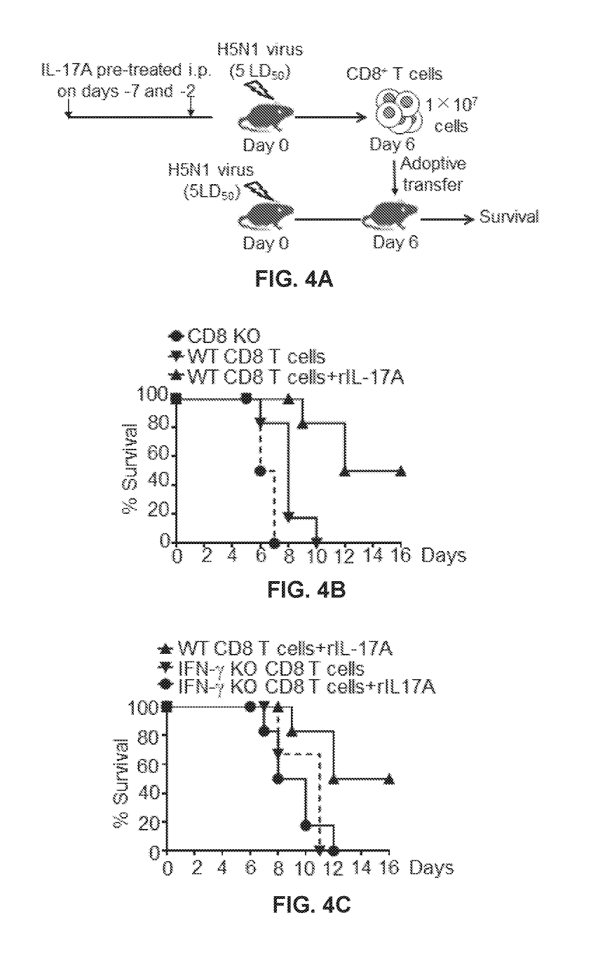

As described in more detail below, adoptive transfer was used to further establish that the increased immune response supported by the vaccine including IL-17A as an adjuvant was facilitated by IFN-γ producing CD8⁺ T cells. The scheme for adoptive transfer of CD8⁺ T cells is illustrated in FIG. 4A.

In particular, wild-type CD8⁺ T cells from rIL-17A pre-treated, H5N1 infected mice or untreated, H5N1 infected mice were adoptively transferred into H5N1 infected, wild-type mice. Briefly, a first group of C57BL/6 (i.e., wild-type) mice was pre-treated i.p. with 0.5 μg of rIL-17A on day 7 and day 2 before viral infection. A second group of C57BL/6 mice was not pre-treated with rIL-17A. A third group of mice, namely mice lacking CD8⁺ T cells (i.e., CD8 knockout mice), was used as a control and also was not pre-treated with rIL-17A. The three groups of C57BL/6 mice were challenged with 5 LD₅₀ of influenza A virus H5N1 on day 0. C57BL/6 mice that were to receive the adoptive transfer (i.e., recipient mice) were also challenged with 5 LD₅₀ of influenza A virus H5N1 on day 0. The H5N1 virus was administered nasally to the mice.

At 6 dpi, CD8⁺ T cells were isolated from the C57BL/6 mice pre-treated with rIL-17A (i.e., the first group of mice above) and from the C57BL/6 mice that did not receive rIL-17A pre-treatment (i.e., the second group of mice above). 1×10⁷ of the CD8⁺ T cells isolated from the first group of mice were then adoptively transferred into C57BL/6 (i.e., wild-type) mice that had been infected with influenza A virus H5N1 for 6 days and mortality was monitored daily until 16 dpi for these recipient mice (i.e., WT CD8 T cells+rIL-17A in FIG. 4B). Additionally, 1×10⁷ of the CD8⁺ T cells isolated from the second group of mice were adoptively transferred into C57BL/6 (i.e., wild-type) mice that had been infected with influenza A virus H5N1 for 6 days and mortality was monitored daily until 16 dpi for these recipient mice (i.e., WT CD8+ T cells in FIG. 4B). The CD8 knockout mice were also monitored daily for mortality until 16 dpi (i.e., CD8 KO in FIG. 4B).

As shown in FIG. 4B (and observed in Example 3 and FIG. 3B), the CD8 knockout mice died at 7 dpi and served as a control for mice lacking CD8⁺ T cells. The data in FIG. 4B are representative of three independent experiments. The infected C57BL/6 mice, which received CD8⁺ T cells from the second group of mice (i.e., C57BL/6 mice not receiving rIL-17A pre-treatment, but challenged with influenza A H5N1 virus), died at 10 dpi (FIG. 4B, WT CD8 T cells). In contrast, 50% of the infected C57BL/6 mice, which received CD8⁺ T cells from the first group of mice (i.e., C57BL/6 mice pre-treated with rIL-17A before challenge with influenza A H5N1 virus), survived at 16 dpi (FIG. 4B, WT CD8 T cells+rIL-17A). These data indicated that the CD8⁺ T cells isolated from mice pre-treated with rIL-17A provided the recipient mice an increased immune response to the virus. These data also indicated that CD8⁺ T cells, upon rIL-17A treatment, were able to provide an increased immune response to the influenza A virus, even in mice already infected with the virus.

Additionally, CD8⁺ T cells from IFN-γ knockout mice receiving or not receiving rIL-17A pre-treatment before viral challenge were adoptively transferred into wild-type, H5N1 infected mice to further establish that IFN-γ producing CD8⁺ T cells facilitated the increased immune response supported by the vaccine including rIL-17A as an adjuvant. Briefly, C57BL/6 (i.e., wild-type) mice were pre-treated i.p. with 0.5 μg of rIL-17A on day 7 and day 2 before viral infection. A first group of IFN-γ knockout mice was also pre-treated i.p. with 0.5 μg of rIL-17A on day 7 and day 2 before viral infection. A second group of IFN-γ knockout mice was not pre-treated with rIL-17A. The two groups of IFN-γ knockout mice and the rIL-17A pre-treated C57BL/6 mice were challenged with 5 LD₅₀ of influenza A virus H5N1 on day 0. C57BL/6 (i.e., wild-type) mice that were to receive the adoptive transfer (i.e., recipient mice) were also challenged with 5 LD₅₀ of influenza A virus H5N1 on day 0. The H5N1 virus was administered nasally to the mice.

At 6dpi, CD8⁺ T cells were isolated from the C57BL/6 mice pre-treated with rIL-17A, and 1×10⁷ of these isolated cells were adoptively transferred into C57BL/6 mice that had been infected with influenza A virus H5N1 for 6 days. The mortality of these recipient mice (i.e., WT CD8 T cells+rIL-17A in FIG. 4C) was monitored daily until 16 dpi.

Also at 6 dpi, CD8⁺ T cells were isolated from the IFN-γ knockout mice pre-treated with rIL-17A (i.e., the first group of IFN-γ knockout mice above) and from the IFN-γ knockout mice that did not receive rIL-17A pre-treatment (i.e., the second group of IFN-γ knockout mice above). 1×10⁷ of the CD8⁺ T cells isolated from the first group of IFN-γ knockout mice were then adoptively transferred into C57BL/6 (i.e., wild-type) mice that had been infected with influenza A virus H5N1 for 6 days. The mortality of these recipient mice was monitored daily until 16 dpi (i.e., IFN-γ KO CD8 T cells+rIL-17A in FIG. 4C). Additionally, 1×10⁷ of the CD8⁺ T cells isolated from the second group of IFN-γ knockout mice were adoptively transferred into C57BL/6 (i.e., wild-type mice) that had been infected with influenza A virus H5N1 for 6 days. For these recipient mice, mortality was monitored daily until 16 dpi (i.e., IFN-γ KO CD8 T cells in FIG. 4C).

As shown in FIG. 4C (and observed in FIG. 4B), 50% of the infected C57BL/6 mice, which received CD8⁺ T cells from rIL-17A pre-treated, infected C57BL/6 mice, survived at 16 dpi (i.e., WT CD8 T cells+rIL-17A in FIG. 4C). This data was agreement with the observation described above and shown in FIG. 4B, and thus, served as a positive control for the adoptive transfer experiment of FIG. 4C. The data in FIG. 4C are representative of three independent experiments.

The infected C57BL/6 mice, which received CD8⁺ T cells from the second group of IFN-γ knockout mice (i.e., IFN-γ knockout mice not receiving rIL-17A treatment before viral challenge), died at 11 dpi (FIG. 4C, IFN-γ KO CD8 T cells). This data indicated that CD8⁺ T cells, which are unable to produce IFN-γ and were derived from mice that did not receive rIL-17 pre-treatment, were not able to facilitate the increased immune response directed against influenza A H5N1 viral infection. The infected C57BL/6 mice, which received CD8⁺ T cells from the first group of IFN-γ knockout mice (i.e., IFN-y knockout mice pre-treated with rIL-17A before viral challenge), died at 12 dpi (FIG. 4C, IFN-γ KO CD8 T cells+rIL-17A). This data indicated that CD8⁺ T cells, which are unable to produce IFN-γ and were derived from mice pre-treated with rIL-17A, were not able to facilitate the increased immune directed against influenza A H5N1 viral infection. Accordingly, the increased immune response supported by the vaccine including rIL-17A as an adjuvant was facilitated by IFN-γ-producing CD8⁺ T cells, and this increased immune response was transferrable (via transfer of the IFN-γ-producing CD8+ T cells exposed to the vaccine including rIL-17A and virus) to mice having an established viral infection.

In summary, the data above indicated that H5N1 infected mice recovered and survived after receiving IFN-γ producing CD8⁺ T cells from rIL-17A pre-treated mice. H5N1 infected mice that received CD8⁺ T cells unable to produce IFN-γ did not recover from and survive viral infection (regardless of rIL-17A pre-treatment). Accordingly, IFN-γ producing CD8⁺ T cells, but not those CD8⁺ T cells unable to produce IFN-γ, facilitated the increased immune response supported by the vaccine including IL-17A as an adjuvant. CD8⁺ T cells and the IFN-γ produced from these CD8⁺ T cells, significantly contributed to the increased immune response provided by the vaccine including IL-17A as an adjuvant.

### Example 5

### Lytic Activity of CD8⁺ T cells

CD8⁺ T cells that are cytolytic are also known as cytolytic T lymphocytes (CTLs). CTLs can have anti-viral activity. Accordingly, the IFN-γ producing CD8⁺ T cells that facilitated the increased immune response supported by the vaccine including IL-17A as an adjuvant were examined for cytolytic activity. In particular, in vivo and in vitro assays for cytolytic activity were used as described below.

With regards to the in vivo assay for cytolytic activity, syngeneic splenocytes were prepared from naïve C57BL/6 mice (i.e., mice not receiving rIL-17A pre-treatment nor challenged with influenza virus). The syngeneic splenocytes were divided into two groups. The first group of syngeneic splenocytes was pulsed with inactivated influenza A virus H5N1 and labeled with 10 μM of carboxyfluorescein succinimidyl ester (CFSE, a fluorescent dye for staining cells). The second group of syngeneic splenocytes was labeled with 0.5 μM of CFSE. Equal numbers of splenocytes from the two groups were mixed together.

Wild-type (i.e., C57BL/6) mice and mice lacking CD8⁺ T cells (i.e., CD8 knockout mice) were pre-treated i.p. with 0.5 μg of rIL-17A on day 7 and day 2 before administration of the virus. A second group of C57BL/6 mice and a second group of CD8 knockout mice did not receive rIL-17A pre-treatment. The four groups of mice were challenged with 5 LD₅₀ of influenza A virus H5N1 on day 0. The H5N1 virus was administered nasally to the mice. At 6 dpi, the mixed splenocytes described above were injected intravenously into these four groups of mice. Eight hours later, splenocytes were isolated from the recipient mice to determine the level of antigen-specific cytolysis in vivo.

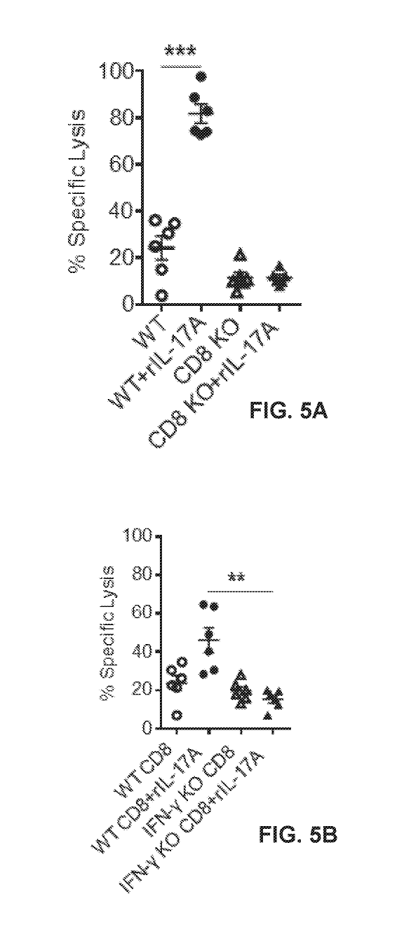

As shown in FIG. 5A, antigen-specific lysis was significantly increased in wild-type mice pre-treated with rIL-17A as compared to wild-type mice that did not receive rIL-17A pre-treatment (i.e., WT+rIL-17A and WT, respectively, in FIG. 5A). The data in FIG. 5A are representative of three independent experiments, mean±SEM, and ***p<0.001 (unpaired student's t-test). Specifically, antigen-specific lysis in rIL-17A pre-treated, wild-type mice was about 4-fold higher than antigen-specific lysis in wild-type mice that did not receive rIL-17A pre-treatment. This data indicated that rIL-17A pre-treatment significantly induced CTL activity. In other words, the vaccine including rIL-17A as an adjuvant significantly induced CTL activity as compared to the vaccine not including rIL-17A.

Additionally, CD8 knockout mice receiving and not receiving rIL-17A pre-treatment had similar levels of antigen-specific cytolytic activity (FIG. 5A, CD8 KO+rIL-17A and CD8 KO, respectively). Rather, CD8 knockout mice, regardless of rIL-17A pre-treatment, had levels of antigen-specific lysis that were lower than the levels of antigen-specific lysis observed for wild-type mice. Accordingly, CTL activity was decreased about 2-fold in both CD8 knockout mice receiving and not receiving rIL-17A pre-treatment as compared to the CTL activity of wild-type mice. In summary, these data indicated that CD8⁺ T cells were required for cytolytic activity and this cytolytic activity was significantly increased in response to rIL-17A pre-treatment.

To further examine the cytolytic activity of the CD8⁺ T cells and to determine if IFN-γ production was needed for cytolytic activity, an in vitro assay was used to assess the cytolytic activity of different populations of CD8⁺ T cells. In particular, wild-type and IFN-γ knockout mice were pre-treated i.p. with 0.5 μg of rIL-17A on day 7 and day 2 before administration of the virus. A second group of wild-type mice and a second group of IFN-γ knockout mice did not receive rIL-17A pre-treatment. The four groups of mice were challenged with 5 LD₅₀ of influenza A virus H5N1 on day 0. The virus was administered nasally to the mice. At 6 dpi, CD8⁺ T cells were isolated from the four groups of mice and used as effector T cells.

Syngeneic splenocytes were prepared from naive C57BL/6 mice (i.e., mice not receiving rIL-17A pre-treatment nor challenged with influenza virus). The syngeneic splenocytes were divided into two groups. The first group of syngeneic splenocytes was pulsed with inactivated influenza A virus H5N1 and labeled with 10 μM of CF SE. The second group of syngeneic splenocytes was labeled with 0.5 μM of CFSE and served as a negative control.

The first and second groups of splenocytes were then independently mixed with the effector T cells isolated from each of the four groups of mice. After 3 days, specific lysis was analyzed by flow cytometry.

As shown in FIG. 5B, CD8⁺ T cells isolated from wild-type mice pre-treated with rIL-17A had higher levels of antigen-specific lysis as compared to the CD8⁺ T cells isolated from wild-type mice that did not receive rIL-17A pre-treatment (WT CD8+rIL-17A and WT CD8, respectively, in FIG. 5B). The data in FIG. 5B are representative of three independent experiments, mean±SEM, and **p<0.01 (unpaired student's t-test). This data indicated that rIL-17A pre-treatment increased the cytolytic activity of CD8⁺ T cells as was also observed in the in vivo assay discussed above (FIGS. 5A and 5B). CD8⁺ T cells incapable of producing IFN-γ had significantly reduced levels of antigen-specific lytic activity as compared to CD8⁺ T cells isolated from rIL-17A pre-treated, wild-type mice (FIG. 5B, IFN-γ KO CD8 and WT CD8+rIL-17A, respectively). Additionally, CD8⁺ T cells obtained from rIL-17A pre-treated, IFN-γ knockout mice had similar levels of antigen-specific lytic activity as the CD8⁺ T cells isolated from IFN-γ knockout mice that did not receive rIL-17A pre-treatment (FIG. 5B, IFN-γ KO CD8+rIL-17A and IFN-γ KO CD8, respectively). CTL activity of the CD8⁺ T cells isolated IFN-γ knockout mice that did and did not receive rIL-17A pre-treatment was reduced about 3-fold and about 2-fold, respectively, as compared to the CTL activity of CD8⁺ T cells isolated from rIL-17A pre-treated, wild-type mice. These data indicated that the cytolytic activity of CD8⁺ T cells was significantly impaired in the IFN-γ knockout mice as compared to wild-type mice. These data also indicated that without IFN-γ production, the cytolytic activity of CD8⁺ T cells was not significantly induced by rIL-17A pre-treatment.

In summary, the data obtained from the in vivo and in vitro antigen-specific lysis assays demonstrated that the cytolytic activity of CD8⁺ T cells induced by rIL-17A pre-treatment was dependent upon IFN-γ production.

Accordingly, these data, along with the data from the above Examples, demonstrated that the vaccine including IL-17A as an adjuvant (i.e., pre-treatment with rIL-17A before administration of the virus) increased the immune response to the virus as compared to the vaccine not including IL-17A (i.e., no IL-17A pre-treatment before administration of the virus). This increased immune response provided by the vaccine including IL-17A as an adjuvant protected mice from death in a dose dependent manner.

The increased immune response provided by the vaccine including IL-17A as an adjuvant was facilitated by IFN-γ producing CD8⁺ T cells that had antigen-specific cytolytic activity. These IFN-γ producing cytotoxic CD8⁺ T cells were induced by the vaccine including IL-17A as an adjuvant (i.e., by IL-17A pre-treatment). In summary, the vaccine including IL-17A as an adjuvant significantly induced an anti-viral immune response that in turn protected (and treated) the subject receiving such a vaccine from influenza viral infection.

### Example 6

### IL-17A Protein Production for Use in Examples 7-12

cDNAs encoding swine, bovine, avian, murine, and human IL-17A were subcloned into the pET28a vector. Each cDNA was codon optimized. The resulting constructs were transformed into BL21 E. coli. For each transformation, the highest expressing IL-17A clone was chosen and mast seeds were made from the chosen clones. Small scale cultivation and IPTG induction time studies were conducted to determine the conditions for protein expression and subsequent purification. FIGS. 6-17 show mRNA, coding, amino acid and nucleic acid sequences used in the studies.

Medium scale fermentation (10 liters) was then conducted with the optimized IPTG induction determined from the small scale study. Bacteria were harvested and IL-17A was extracted and purified from these bacteria. During these medium scale fermentation studies, a native folding process for IL-17A was developed.

After the conclusion of these medium scale fermentation studies, large scale fermentation (50 liter) was conducted to produce IL-17A protein. Quality control studies were conducted on the purified and folded IL-17A, namely concentration, purity, endotoxin, and in vitro biological activity.

### Example 7

### Effect of IL-17A on an Inactivated Vaccine Against Porcine Reproductive and Respiratory Syndrome (PRRS) in Mice

FIGS. 9 and 14-15 show mRNA, coding, amino acid and nucleic acid sequences used in the studies. The sequences used in Example 7 for IL-17 are specific for swine species.

As described above, IL-17A, when included as an adjuvant in a vaccine for influenza virus, provided an enhanced or increased immune response as compared to the vaccine not including IL-17A as an adjuvant. To further examine the ability of IL-17A to serve as an adjuvant, IL-17A was included in an inactivated vaccine against Porcine Reproductive and Respiratory Syndrome (PRRS) that was administered to mice. Specifically, Kunming white mice, aged 6 to 8 weeks, were placed into groups. There were four animals per group. The first group received a vaccine including inactivated PRRS virus (JXAI-R strain) emulsified in oil. The second group of mice received a vaccine including inactivated PRRS virus (JXAI-R strain) emulsified in oil and 0.45 μg/dose IL-17A protein. The third group of mice received a vaccine including inactivated PRRS virus (JXAI-R strain) emulsified in oil and 0.15 μg/dose IL-17A protein. The fourth group of mice received a vaccine including inactivated PRRS virus (JXAI-R) strain emulsified in oil and 0.05 μg/dose IL-17A protein. The vaccines were administered via intramuscular injection in the leg. Each injection was 100 μl and each animal received a single immunization.

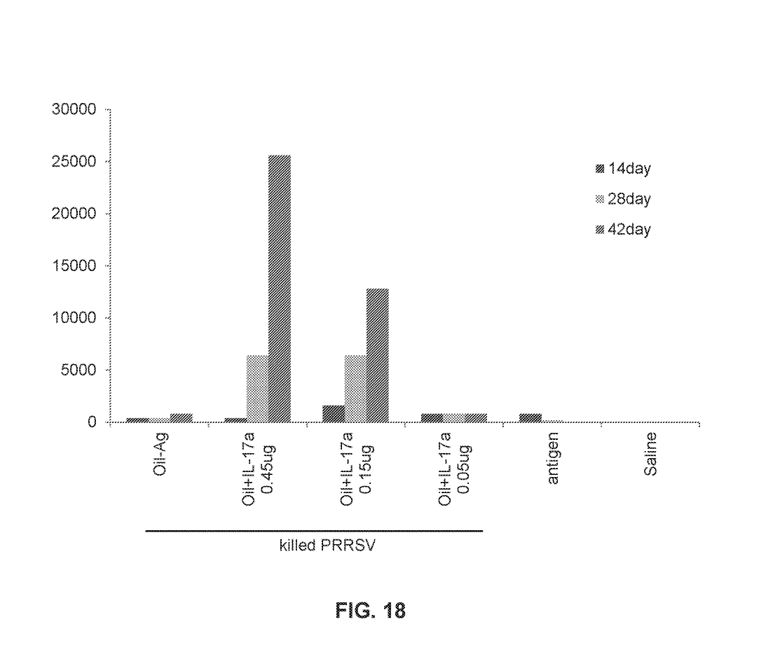

Serum samples were collected from each mouse at days—1, 14, 28, and 42, in which the mice were immunized at day 0. The sera were tested in an ELISA to determine antibody titer. The antigen used in the ELISA assay was PRRS killed virus. The results of this ELISA are shown in FIG. 18.

These data indicated that antibody titers were higher in those animals that received the vaccine including IL-17A as compared to the animals that received the vaccine not including IL-17A. Additionally, this increased antibody titer was dependent on the dose of IL-17A present in the vaccine. 0.45 μg IL-17A provided a larger increase in antibody titer than 0.15 μg IL-17A, which in turn, provided a larger increase in antibody titer than 0.05 μg IL-17A. Accordingly, these data demonstrated that IL-17A, when included in the vaccine, increased the titer of antibody reactive against the virus as compared to the vaccine not including IL-17A. These data also demonstrated that the adjuvant effect of IL-17A is not specific to influenza virus, but also increased the immune response to other viruses, e.g., PRRS virus.

### Example 8

### Effect of IL-17A on an Inactivated Vaccine Against Porcine Reproductive and Respiratory Syndrome (PRRS) in Piglets

FIGS. 9 and 14-15 show mRNA, coding, amino acid and nucleic acid sequences used in the studies.

As described above, inclusion of IL-17A in an inactivated PRRS vaccine increased the immune response in mice as compared to the vaccine not including IL-17A. These studies were extended into piglets. Specifically, 35 piglets aged about 30 days that were negative for PRRS virus were divided into 7 groups. The piglets were determined to be negative for PRRS virus via a reverse transcriptase polymerase chain reaction (RT-PCR) assay that detected PRRS virus antigen and an ELISA that detected antibodies directed against PRRS virus. Each group had five animals. The first group of animals received a vaccine including inactivated PRRS virus and 3 μg IL-17A protein. The second group of animals received a vaccine including inactivated PRRS virus and 9 μg IL-17A protein. The third group of animals received a vaccine including inactivated PRRS virus and 27 μg IL-17A protein. The fourth group of animals received a vaccine including PRRS virus antigen and 9 μg IL-17A protein. The antigen was a PRRS formalin killed virus. The fifth group of animals received a vaccine including killed PRRS virus, i.e., a DHN (Dahuanong Veterinary Animal Health Corporation, a Chinese veterinary vaccine company) inactivated vaccine. The sixth group of animals received saline. The seventh group of mice received a vaccine including attenuated PRRS virus, i.e., a DHN live attenuated vaccine. The PRRS virus was JXAI-R strain.

Each animal received a single immunization of its respective vaccine, which was administered via intramuscular injection in the neck or ear in a 2 mL dose. Each animal was observed for at least three consecutive days following immunization. Observations included recordation of each animal's body temperature, feed intake, and mental status.

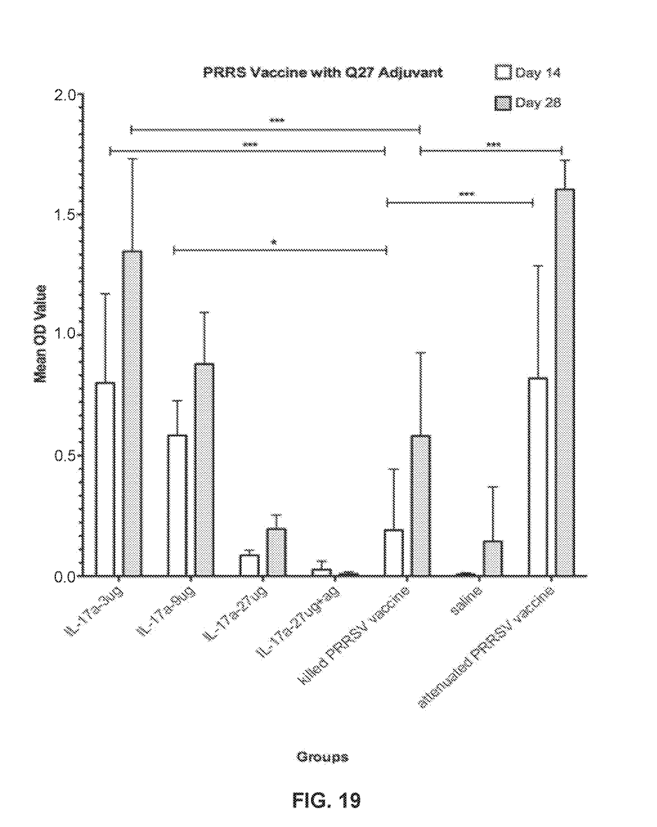

Serum samples were taken prior to immunization and after immunization at days 14 and 28. Not less than 5 mL of serum was taken for each sample. Antibodies directed to PRRS virus were detected in the sera by an indirect ELISA. The results from the ELISA analysis are shown in FIG. 19. The vaccines including 3 μg or 9 μg of IL-17A induced significantly higher levels of anti-PRRS virus antibodies as compared to the vaccine including only killed PRRS virus (i.e., Group 5). The antibody response induced by the vaccine including 3 μg IL-17A was comparable to the antibody response induced by the vaccine including attenuated PRRS virus (i.e., Group 7).

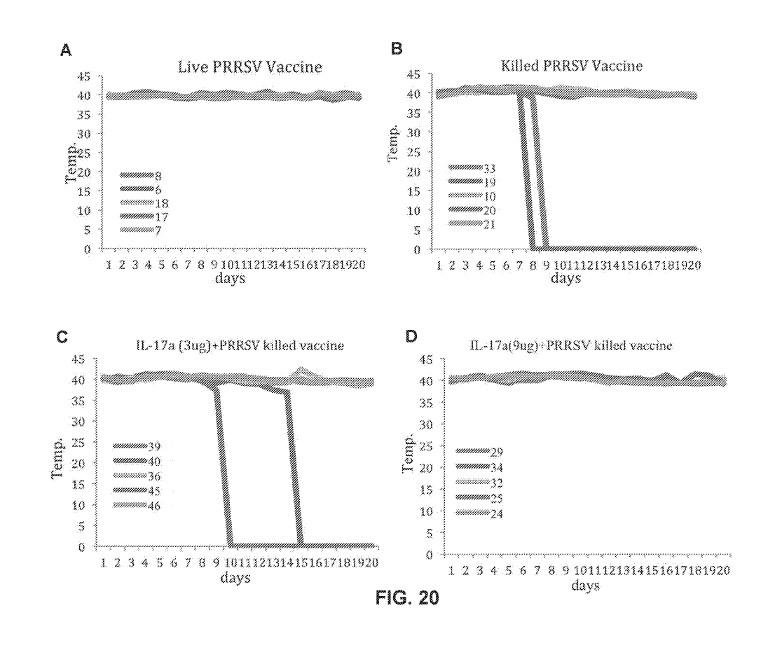

The first, second, fifth, and seventh groups of immunized mice were also challenged with the NVDC-JXA1 strain of PRRS virus. Specifically, animals were challenged with a virus titer of greater than or equal to 10^{4.5} TCID₅₀/mL in a 3 mL dose. The virus was administered via the ear muscle. After viral challenge, animals were observed daily for 21 days along with measurement of the daily temperature. The results of this daily temperature measurement are shown in FIG. 20. Additionally, in the group immunized with killed PRRS virus (i.e., group 5), two animals died, and thus, this vaccine provided the weakest protection of the four vaccines examined in this study (FIG. 20B, see lines at dropped to 0). Two animals also died in the group immunized with inactivated PRRS virus and 3 μg IL-17A (i.e., group 1; FIG. 20C, see lines that dropped to 0), but at a later time point as compared to the group 5 animals. Accordingly, the vaccine including inactivated PRRS virus and 3 μg IL-17A provided greater protection against viral challenge as compared to the vaccine including only killed PRRS virus. Increased protection to viral challenge was provided by the vaccine including inactivated PRRS virus and 9 μg IL-17A, and the vaccine including only attenuated PRRS virus. These two vaccines provided similar efficacy in protecting the animals from viral challenge (FIGS. 20A and 20D).

In summary, the above data demonstrated that IL-17A, when included in the vaccine, increased the antibody response to the virus and provided increased protection to viral challenge as compared to the vaccine not including IL-17A.

### Example 9

### Effect of IL-17A on a Vaccine for Porcine Circovirus Type 2

FIGS. 6 and 12 show mRNA, coding, amino acid and nucleic acid sequences used in the studies.

As described above, inclusion of IL-17A in vaccines for influenza virus and PRRS virus increased the immune response and protection against viral infection as compared to the vaccines not including IL-17A. This adjuvant effect of IL-17A was further examined in mice with regards to vaccines for porcine circovirus type 2 (PCV2). Specifically, 6 to 8 week old healthy female Kunming mice that were negative for antibodies directed against PCV2 as measured by ELISA were divided into groups. If the ELISA for PCV2 antibody did not exceed 1:50, the animals were considered negative for PCV2. Each group had four mice. The first group was administered PCV2 vaccine with A15 adjuvant. The A15 adjuvant is an adjuvant produced and sold by Seppic company (a franchise company). The second group of mice were administered PCV2 vaccine with 0.05 μg/dose IL-17A protein. The third group of mice were administered PCV2 vaccine with 0.15 μg/dose IL-17A protein. The fourth group of mice were administered PCV2 vaccine with 0.45 μg/dose IL-17A protein. The fifth group of mice were administered PCV2 antigen without any adjuvant. The sixth group of mice were administered saline. The PCV2 antigen is one antigen that killed PCV2 virus. The PCV2 vaccine comprises the PCV2 antigen and at lease one adjuvant. In one specific embodiment, the adjuvant is A15.

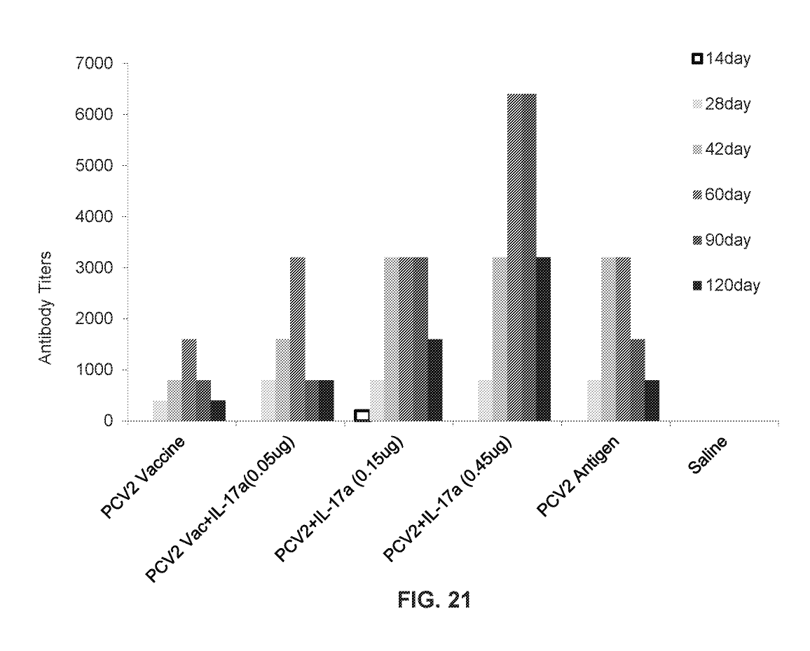

Each animal received a single immunization in the leg muscle at 0.1 mL/dose. The antigen was PCV2 LG strains. Each animal was observed for at least three consecutive days following immunization. Observations examined the injection sites and animal movement. Serum were obtained from each animal at days 0, 14, 28, 42, 60, 90, and 120. Immunization occurred on day 0. Each serum sample was not less than 100 μL to 200 μL. Antibodies to PCV2 in the sera were measured by indirect ELISA. The results of this ELISA analysis are shown in FIG. 21.

These data indicated that the antibody titer was greater in the animals receiving a dose of IL-17A in combination with PCV2 vaccine (i.e., groups 2, 3, and 4) as compared to the antibody titer measured in the animals receiving PCV2 vaccine (i.e., group 1). Specifically, the 0.45 μg IL-17A induced the highest level of antibodies against PCV2. Additionally, the duration of the antibody response was significantly longer in the groups administered PCV2 vaccine in combination with IL-17A as compared to the group administered PCV2 vaccine (i.e., group 1). Specifically, the 0.45 μg IL-17a induced the longest or most durable level of anti-PCV2 antibody. In summary, these data demonstrated that inclusion of IL-17A in the vaccine increased the immune response (e.g., antibody titer and duration of antibody response) as compared to the vaccine not including IL-17A.

### Example 10

### Effect of IL-17A on a Vaccine for Porcine Circovirus Type 2

FIGS. 6 and 12 show mRNA, coding, amino acid and nucleic acid sequences used in the studies.

As described above, IL-17A in combination with PCV2 vaccine provided an increased immune response as compared to PCV2 vaccine alone. These studies with PCV2 vaccine and IL-17A were extended into BALB/C mice. 5 week old female BALB/C mice that were negative for PCV2 as measured by ELISA for PCV2 antibody were divided into groups. Mice were determined to be negative for PCV2 when the PCV2 antibody titer was not higher than 1:50 in the ELISA. There were 6 or 7 mice in each group.

Group 1 mice received PCV2 vaccine subcutaneously. The PCV2 vaccine was PCV2 antigen and 15A adjuvant. The PCV2 antigen was a PCV2 LG strain, specifically, batch 121129, IPMA at 10^{5.25}TCID₅₀/ml. Group 2 mice received the combination of PCV2 vaccine and IL-17A subcutaneously. Group 3 mice received PCV2 vaccine intramuscularly. Group 4 mice received the combination of PCV2 vaccine and IL-17A intramuscularly. Group 5 mice received saline. Each animal was immunized with a 0.5 ml dose of its respective vaccine in each immunization. Each animal received two immunizations, in which the first immunization was at day 0 and the second immunization was at day 21.

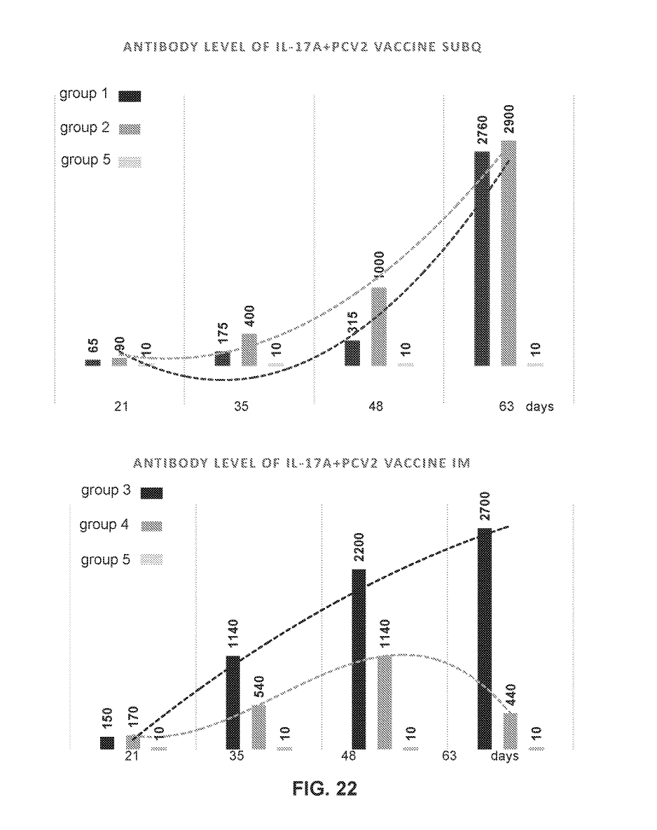

After immunization, animals were observed continuously for at least three days. Specifically, the injection site and the mental condition of each animal were observed. Serum samples were collected from each animal 1 day before immunization and at days 21, 35, 49, and 63 after the first immunization. Antibodies were detected by indirect immunoperoxidase monolayer assay (IPMA) and indirect ELISA test. The antibody levels for each of immunization groups 1, 2, 3, 4, and 5 are shown below in Table 1 and FIG. 22. FIG. 22 is a graphic illustration of the data presented in Table 1 divided into intramuscular and subcutaneous immunizations.

The data in Table 1 and FIG. 22 indicated that inclusion of IL-17A in the vaccine as an adjuvant enhanced antibody titer when the vaccine was administered subcutaneously. IL-17A was a less effective adjuvant when the vaccine was administered intramuscularly. In summary, these data showed that the vaccine including IL-17A and administered subcutaneously elicited higher antibody titers as compared to the vaccine not including IL-17A and administered subcutaneously. These data also demonstrated that IL-17A exerted its adjuvant effect in a tissue specific manner with regards to the PCV2 vaccine, i.e., higher titers were elicited via the subcutaneous route of administration than the intramuscular route of administration.

### Example 11

### Effect of IL-17A on a Swine Foot and Mouth Disease (FMD) Inactivated Vaccine

FIGS. 9 and 15 show mRNA, coding, amino acid and nucleic acid sequences used in the studies.

As described above, IL-17A, when included in the vaccine, increased the immune response for influenza, PRRS virus, and PCV2 vaccines. To further examine the adjuvant effect of IL-17A, the foot and mouth disease (FMD) inactivated vaccine was examined in 6 to 8 week old Kunming mice. Specifically, the FMD inactivated vaccine was killed swine FMD type O vaccine. This was a vaccine against swine FMD type O (OZK/93) strains. The vaccine was produced by China Animal Husbandry Industry Limited and Share Ltd Lanzhou pharmaceutical factory. It was vaccine batch 1009002.

Each group had three mice. Group 1 received the above-described FMD inactivated vaccine. Group 2 received the FMD inactivated vaccine and IL-17A (0.45 μg/dose). Group 3 received the FMD inactivated vaccine and IL-17A (0.15 μg/dose). Group 4 received the inactivated vaccine and IL-17A (0.05 μg/dose). Group 5 received saline. Each animal was administered its respective immunization via intramuscular injection. Each injection was 100 μl. Each animal received a single immunization.

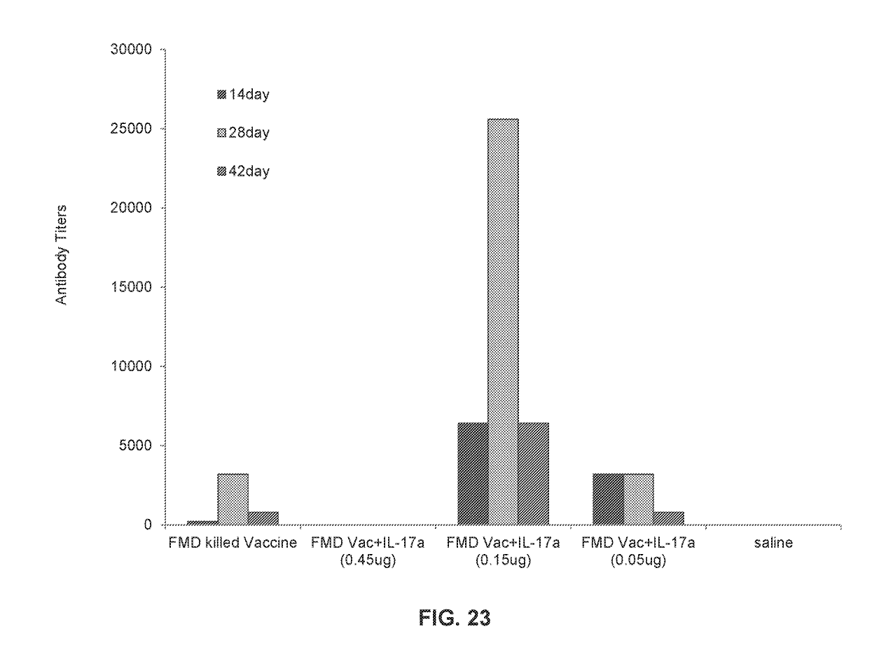

Serum samples were taken from each animal before immunization on day 0. Serum samples were also taken from each animal on days 14, 28, and 42. Antibody titers were measured by an ELISA assay and a pig FMD type O VP1 structural protein liquid-phase blocking ELISA antibody test. The results from these measurements are shown in FIG. 23.

These results demonstrated that inclusion of IL-17A in the immunization increased the immunogenicity of the antigen with respect to antibody titer and the duration of the antibody response. Specifically, the level of anti-FMD virus antibody was higher in the groups receiving IL-17A in combination with the FMD vaccine as compared to the group that received the FMD vaccine alone. IL-17A at 0.15 μg/dose induced the highest level of anti-FMD virus antibody of the three doses of IL-17A examined in this study.

Additionally, the duration of the antibody response to the FMD vaccine was longer when IL-17A was included in the immunization as compared to when IL-17A was not included in the immunization. IL-17A at 0.15 μg/dose induced a durable level of the anti-FMD virus antibody.

In summary, these data indicated that inclusion of IL-17A in a vaccine elicited an increased immune response (e.g., antibody titer and duration) as compared to the vaccine that did not include IL-17A. These data also further indicated that IL-17A provided its adjuvant effect independent of the identity of the immunogen because increased immune responses were observed when IL-17A was included in vaccinations for influenza virus, PRRS virus, PCV2, and FMD virus.

### Example 12

### Effect of IL-17A on Porcine Actinobacillus pleuropneumoniae (APX I, II, and III) Vaccines

As described above, inclusion of IL-17A in a vaccine increased the immune response to the immunogen as compared to the vaccine that did not include IL-17A. To further examine the adjuvant effect of IL-17A, 14 adjuvants were studied for their effect on porcine Actinobacillus pleuropneumonias vaccines (i.e., Apx I, Apx II, and Apx III).

Specifically, the pig study included two groups:

The first group was vaccinated with a formulation containing saline, Alugel, and the antigen (i.e., Apx I, Apx II, or Apx III). AlOH was at 21%.

The second group was vaccinated with a formulation containing saline, Alugel, the antigen (i.e., Apx I, Apx II, or Apx III) and IL-17A. The IL-17A was at 15% and AlOH at 21%. Each immunization group contained five 3-week old piglets that were susceptible to Actinobacillus pleuropneumonias. Each piglet received its respective vaccine via intramuscular vaccination of a 2 ml dose on day 0 and again on day 21. Blood samples were collected at day 21 (after the first immunization). Serological tests were conducted for each sample. The serological tests were for antibodies directed against Apx I, Apx II, or Apx III. The results of these serological tests demonstrated that all pigs vaccinated with a formulation containing IL17 elicited an at least twice higher level of antibody titers than pigs vaccinated without IL17. These results thus show that IL17 could increase by at least 100% the immune response generated against a cell antigen in vivo.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the invention, may be made without departing from the spirit and scope thereof.

1. A vaccine comprising an antigen and IL-17.

2. The vaccine of claim 1, wherein IL-17 is encoded by SEQ ID NO:22 or SEQ ID NO:23.

3. The vaccine of claim 1, wherein IL-17 is a peptide comprising the amino acid sequence as set forth in SEQ ID NO.24.

4. (canceled)

5. The vaccine of claim 1, wherein the antigen is selected from the group consisting of a hepatitis antigen, a human papilloma virus (HPV) antigen, an HIV antigen, an influenza antigen, a Plasmodium falciparum antigen, a porcine reproductive and respiratory syndrome (PRRS) virus antigen, a porcine circovirus type 2 antigen, a foot and mouth disease virus (FMDV) antigen, an Actinobacillus pleuropneumoniae antigen, a Mycoplasma hyopneumoniae antigen and a fragment thereof:
wherein the HPV antigen is selected from the group consisting of HPV16 E6 antigen, HPV16 E7 antigen, and combination thereof;
wherein the HIV antigen is selected from the group consisting of Env A, Env B, Env C, Env D, B Nef-Rev, Gag, and any combination thereof;
wherein the influenza antigen is selected from the group consisting of H1 HA, H2 HA, H3 HA, H5 HA, BHA antigen, and any combination thereof;
wherein the Plasmodium falciparum antigen includes a circumsporozoite (CS) antigen;
and wherein the hepatitis antigen is selected from the group consisting of a HAV antigen, a HBV antigen, a HCV antigen, a HDV antigen, and a HEV antigen.

6. (canceled)

7. (canceled)

8. (canceled)

9. (canceled)

10. (canceled)

11. The vaccine of claim 5, wherein the hepatitis antigen is an antigen from HBV.

12. The vaccine of anyone of claim 5, further comprising a pharmaceutically acceptable excipient.

13. The vaccine of claim 5, wherein the said nucleic acid encoding the antigen and/or said nucleic acid encoding IL-17 are in an expression vector.

14. A method for inducing or increasing an immune response in a subject, the method comprising administering to a subject in need thereof the vaccine of anyone of claim 5.

15. The method of claim 14, wherein administering the vaccine includes electroporation.

16. The method of claim 14, wherein the immune response in the subject is increased by at least about 2-fold.

17. An IL-17 nucleic acid, encoded by SEQ ID Nos. 22 and 23 or protein having a sequence SEQ ID No.24.

18. (canceled)

19. (canceled)

20. (canceled)

21. (canceled)

22. (canceled)

23. (canceled)

24. (canceled)

25. A kit comprising a first and a second container, said first container comprising an antigen and said second container comprising an IL-17 protein or nucleic acid.

26. A kit comprising a first container comprising an antigen in admixture with an IL-17 protein or nucleic acid and a means for administering said admixture to a subject.
